(19) United States
(12) Patent Application Publication
(10) Pub. No.: US 2021/0259615 A1
(43) Pub. Date: Aug. 26, 2021

(54) RESILIENCE TRAINING

(71) Applicant: The Medical Research, Infrastructure and Health Services Fund of the Tel Aviv Medical Center, Tel-Aviv (IL)

(72) Inventors: Talma HENDLER, Tel-Aviv (IL); Gal RAZ, Kfar Daniel (IL); Nimrod Jackob KEYNAN, Givatayim (IL)

(73) Assignee: The Medical Research, Infrastructure and Health Services Fund of the Tel Aviv Medical Center, Tel-Aviv (IL)

(21) Appl. No.: 17/319,265

(22) Filed: May 13, 2021

Related U.S. Application Data
(63) Continuation of application No. PCT/IL2019/051245, filed on Nov. 14, 2019.
(60) Provisional application No. 62/767,650, filed on Nov. 15, 2018.

(51) Int. Cl. A61B 5/375; A61B 5/00; A61B 5/291; A61B 5/386; A61B 5/384; A61B 5/377; G16H 20/70; G16H 40/60; G01R 33/48

(52) U.S. Cl. CPC A61B 5/375; A61B 5/4884; A61B 5/291; A61B 5/386; A61B 5/384; A61B 5/055; A61B 5/377; G16H 20/70; G16H 40/60; G01R 33/4806; G01R 33/4808; A61B 5/7267

(57) ABSTRACT

A method for resilience training, including:
exposing a healthy human subject to one or more stress-evoking perturbations selected to affect activation of deeply located limbic areas;
instructing the healthy human subject to perform in a timed relation to the exposing, at least one activity configured to selectively affect activation of said deeply located limbic areas;
recording EEG signals from the healthy human subject during the exposing;
analyzing the recorded EEG signals to identify at least one EEG signature indicating an activation level of the deeply located limbic areas;
determining an activation level of the deeply located limbic areas based on the identified at least one EEG signature;
delivering a human-detectable indication to the healthy human subject according to the determined activation level.

### RELATED APPLICATIONS

This application is a Continuation of PCT Patent Application No. PCT/IL2019/051245 having International filing date of Nov. 14, 2019, which claims the benefit of priority under USC § 119(e) of U.S. Provisional Patent Application No. 62/767,650 filed on Nov. 15, 2018. The contents of the above applications are all incorporated by reference as if fully set forth herein in their entirety.

### FIELD AND BACKGROUND OF THE INVENTION

The present invention, in some embodiments thereof, relates to emotion regulation training and, more particularly, but not exclusively, to stress regulation training.

### SUMMARY OF THE INVENTION

Some examples of some embodiments of the invention are listed below:

Example 1. A method for resilience training, comprising:

exposing a healthy human subject to one or more stress-evoking perturbations selected to affect activation of deeply located limbic areas;

performing in a timed relation to said exposing one or more mental or physical activities configured to affect activation of said deeply located limbic areas;

measuring EEG signals from said healthy human subject during said exposing;

analyzing said recorded EEG signals to identify signals related to activation of said deeply located limbic areas;

determining an activation level of said deeply located limbic areas based on said identified signals;

delivering a human-detectable indication to said healthy human subject according to said determined activation level.

Example 2. A method according to example 1, wherein said healthy human subject is a subject having cortisone levels within a normal range of values.

Example 3. A method according to any one of the previous examples, wherein said healthy human subject is a subject having a stress-indicating physiological factor within normal range of values.

Example 4. A method according to any one of the previous examples, wherein said one or more stress-evoking perturbations are perturbations selected to induce a stress response in said healthy human subject.

Example 5. A method according to any one of the previous examples, wherein said deeply located limbic areas, are brain regions related to the limbic system located underneath the brain cortex.

Example 6. A method according to any one of the previous examples, wherein said deeply located limbic areas comprise the amygdala.

Example 7. A method according to any one of the previous examples, wherein said time relation comprises prior-to, during and/or after said exposing.

Example 8. A method according to any one of the previous examples, wherein said one or more mental or physical activities activate brain regions or neural circuits which relate to activation control of said deeply located limbic areas;

Example 9. A method according to any one of the previous examples, wherein said activation level is determined based on a relation between said EEG signals and a fingerprint indicative of an activity level.

Example 10. A method of controlling an environment of a healthy human subject, comprising:

exposing a human subject to an environment comprising one or more stress-inducing factors;

providing said human subject with one or more resilience promoting activities configured to self-control activation of one or brain areas, in a timed relation with said exposing.

Example 11. A method according to example 10, wherein said one or more resilience promoting activities are provided during, after or prior to said exposing;

Example 12. A method according to any one of examples 10 or 11, wherein said resilience promoting activities comprise mental exercises and/or physical exercises.

Example 13. A method for controlling resilience training, comprising: selecting healthy human subjects;

instructing said healthy human subjects how to regulate one or more stress-related brain areas using EEG-NF.

Example 14. A method of controlling resilience training, comprising:

(a) selecting healthy human subjects;

(b) providing one or more external stress-inducing factors configured to induce a stress response in said subjects; and

(c) reducing stress by said humans using self-control of one or more brain areas that affect response to stress;

(d) controlling one or both of (b) and (c) to maintain stress within a desired range.

Example 15. A method according to example 14, wherein said desired range is personalized for one or more of said healthy human subjects.

Example 16. A method for resilience assessment, comprising:

exposing a healthy human subject to one or more stress-evoking perturbations selected to induce stress in said subject;

measuring EEG signals from said healthy human subject during said exposing;

analyzing said recorded EEG signals to identify signals related to activation of said deeply located limbic areas;

determining values at least one parameter related to an activation level of stress-related deeply located limbic areas based on said identified signals;

delivering an indication regarding a resilience of said subject based on the determined values.

Example 17. A resilience training system, comprising:

a user interface;

one or more electrodes configured to measure EEG signals;

a control unit electrically connected to said user interface and to said one or more electrodes, wherein said control unit is configured to:

(a) display an interface to a subject by said user interface, wherein said interface follows an activity level of stress-related brain regions of said subject;

(b) provide instructions using said user interface to said subject how to modulate activity of said stress-related brain regions;

(c) measure EEG signals from said human subject by said one or more electrodes;

(d) analyze said EEG signals to determine activity level of said stress-related brain regions;

(e) modify said interface according to said determined activity.

Example 18. A resilience training system, comprising:

a user interface;

one or more electrodes configured to measure EEG signals;

a control unit electrically connected to said user interface and to said one or more electrodes, wherein said control unit is configured to:

(a) deliver one or more stress-evoking perturbations using said user interface to a human subject, wherein said perturbations are selected to affect activation of deeply located limbic areas using said user interface;

(b) provide instructions using said user interface to said human subject to perform one or more of mental or physical activities;

(c) measure EEG signals from said human subject by said one or more electrodes;

(d) analyze said EEG signals to determine whether the measured EEG signals correspond with a desired activation level of said limbic areas;

(e) deliver an indication to said human subject using said user interface based on said analysis results.

Some additional examples of some embodiments of the invention are listed below:

Example 1. A method for resilience training, comprising:

exposing a healthy human subject to one or more stress-evoking perturbations selected to affect activation of deeply located limbic areas;

instructing said healthy human subject to perform in a timed relation to said exposing, at least one activity configured to selectively affect activation of said deeply located limbic areas;

recording EEG signals from said healthy human subject during said exposing;

analyzing said recorded EEG signals to identify at least one EEG signature indicating an activation level of said deeply located limbic areas;

determining an activation level of said deeply located limbic areas based on said identified at least one EEG signature;

delivering a human-detectable indication to said healthy human subject according to said determined activation level.

Example 2. A method according to example 1, wherein said at least one activity comprises at least one mental activity or at least one physical activity.

Example 3. A method according to any one of examples 1 or 2, wherein said recording comprises recording EEG signals from said healthy human subject during and/or following the performing of said one or more activities, and wherein said determining comprises determining an activation level and/or a change in activation level of said of said deeply located based on at least one EEG signature indicating said activation level and/or said change in activation level.

Example 4. A method according to any one of the previous examples, wherein said delivering comprises modifying said one or more stress-evoking perturbations according to said determined activation level.

Example 5. A method according to any one of the previous examples, comprising selecting said at least one activity out of two or more activities based on an ability of said at least one activity to selectively affect activation of said deeply located limbic area when performed by said healthy human subject.

Example 6. A method according to any one of the previous examples, wherein said healthy human subject is a subject having cortisone levels within a normal range of values.

Example 7. A method according to any one of the previous examples, wherein said healthy human subject is a subject having a stress-indicating physiological factor within normal range of values.

Example 8. A method according to any one of the previous examples, wherein said one or more stress-evoking perturbations are perturbations selected to induce a stress response in said healthy human subject.

Example 9. A method according to any one of the previous examples, wherein said deeply located limbic areas, are brain regions related to the limbic system located underneath the brain cortex.

Example 10. A method according to any one of the previous examples, wherein said deeply located limbic areas comprise the amygdala.

Example 11. A method according to any one of the previous examples, wherein said time relation comprises prior-to, during and/or after said exposing.

Example 12. A method according to any one of the previous examples, wherein said at least one activity activates brain regions or neural circuits which relate to activation control of said deeply located limbic areas.

Example 13. A method for controlling resilience training, comprising:

selecting at least one healthy human subject;

identifying one or more stress-related brain areas in said selected healthy human subject, wherein an activity of said one or more stress-related brain areas is known to be selectively affected when exposing healthy human subjects to a specific stressor;

instructing said at least one healthy human subject how to regulate an activity of said one or more stress-related brain areas using an EEG-NF process including at least one session, wherein said EEG-NF comprises performing at least one mental and/or physical exercise before, during and of following an exposure to said specific stressor, wherein said at least one mental and/or physical exercise is configured to regulate said activity of said one or more stress-related brain areas, and receiving a human detectable indication according to an activity level of said one or more stress-related brain areas based on EEG signals recorded from said at least one healthy human subject.

Example 14. A method according to example 13, comprising calculating alexithymia levels of said at least one healthy human subject following said at least one EEG-NF session, and determining an effect of said EEG-NF on regulation of activity of said one or more stress-related brain areas based on said alexithymia levels.

Example 15. A method according to example 14, comprising modifying a protocol or parameters thereof of said EEG-NF, based on said calculated alexithymia levels.

Example 16. A method according to any one of examples 14 or 15, wherein said calculating comprises calculating a decrease in alexithymia levels following said at least one session of said EEG-NF, wherein said decrease in said alexithymia levels indicates modulation of said one or more stress-related brain areas.

Example 17. A method according to any one of examples 14 to 16, wherein said calculating comprises calculating alexithymia levels using a Toronto Alexithymia Scale or variations thereof.

Example 18. A method according to any one of examples 13 to 17, wherein said at least one healthy human subject is a subject having a stress-indicating physiological factor within normal range of values.

Example 19. A method according to any one of examples 13 to 16, wherein said mental and/or physical exercises are exercises known to lower values of at least one physiological parameter upregulated in response to a stressor.

Example 20. A method according to example 19, wherein said at least one physiological parameter comprises one or more of heart rate, blood pressure, skin conductivity, activation level of at least one brain area and activation level of at least one neural pathway.

Example 21. A method according to any one of examples 13 to 20, wherein said stress-related brain areas comprise the amygdala.

Example 22. A method according to any one of examples 13 to 21, wherein said stress-related brain areas comprise limbic areas of the limbic system located underneath the brain cortex.

Example 23. A method according to any one of examples 13 to 22, comprising assessing an alexithymia level of said at least one healthy human subject, and modifying said instructions to said at least one healthy human subject according to results of said assessment.

Example 24. A method according to any one of examples 13 to 23, comprising quantifying a learning model of decision making processes of said at least one healthy human subject, and modifying said instructions to said at least one subject according to results of said learning model quantification.

Example 25. A method according to example 24, wherein said quantifying comprises quantifying said learning model by calculating learning coefficients of model based and model free decision making processes in said at least one healthy human subject.

Example 26. A method according to any one of examples 24 or 25, wherein said quantifying comprises quantifying said learning model using a two-step decision test. Example 27. A method of controlling resilience training, comprising:

(a) selecting at least one healthy human subject;

(b) providing one or more external stress-inducing factors configured to induce a measurable stress response in said at least one healthy human subject, wherein said stress response is measured by measuring values of at least one physiological parameter affected by the stress response; and

(c) reducing said measurable stress response by said at least one healthy human subject using self-control of one or more brain areas that affect response to stress;

(d) controlling one or both of (b) and (c) to maintain the measurable stress response within a desired range.

Example 28. A method according to example 27, wherein said at least one physiological parameter comprises one or more of heart rate, blood pressure, skin conductivity, activation level of at least one brain area and activation level of at least one neural pathway.

Example 29. A method according to any one of examples 27 or 28, wherein said desired range is personalized for said at least one healthy human subject.

Example 30. A method according to example 29, wherein a minimum value of said range is equal or higher from a stress response measured prior to said providing.

Example 31. A method according to any one of examples 27 to 30, wherein said healthy human subjects are subjects having cortisone levels within a normal range of values.

Example 32. A method according to any one of examples 27 to 31, wherein said healthy human subjects are subjects having a stress-indicating physiological factor within normal range of values.

Example 33. A method according to any one of examples 27 to 32, wherein said one or more brain areas comprise the amygdala.

Example 34. A method according to any one of examples 27 to 33, wherein said one or more brain areas comprise limbic areas of the limbic system located underneath the brain cortex.

Example 35. A method for resilience assessment, comprising:

exposing a healthy human subject to one or more stress-evoking perturbations selected to induce a measurable stress response in said subject;

recording EEG signals from said healthy human subject during said exposing;

analyzing said recorded EEG signals to identify at least one EEG signature related to activation of deeply located limbic areas;

determining values of at least one parameter indicating an activation level of stress-related deeply located limbic areas based on said at least one identified EEG signature;

delivering an indication regarding a resilience of said subject based on the determined values.

Example 36. A method according to example 35, comprising:

calculating a resilience score based on an activation level of said stress-related deeply located limbic areas in response to said exposing.

Example 37. A method according to any one of examples 35 or 36, wherein said healthy human subject is a subject having cortisone levels within a normal range of values.

Example 38. A method according to any one of examples 35 to 37, wherein a healthy human subject is a subject having a stress-indicating physiological factor within normal range of values.

Example 39. A method according to any one of examples 35 to 38, comprising performing by said healthy human subject and in a timed relation to said exposing, one or more mental and/or physical exercises configured to affect activation of said deeply located limbic areas, and wherein said recording comprises recording EEG signals from said healthy human subject during said performing.

Example 40. A method according to example 39, wherein said delivering comprises delivering an indication regarding a resilience of said subject based on a change in activation level of said stress-related deeply located limbic areas following said performing of said one or more mental and/or physical exercises.

Example 41. A method according to any one of examples 35 to 40, wherein said resilience is an ability of a subject to resist and/or overcome deleterious short- and or long-term effects associated with a stressor.

Example 42. A method for selection of at least one healthy human subject for resilience training, comprising:

assessing alexithymia level and/or assessing a learning model of decision making processes of said at least one healthy human subject;

selecting at least one of said healthy human subjects to participate in a neurofeedback (NF) resilience training based on results of said assessing.

Example 43. A method according to example 42, wherein said assessing alexithymia level comprises calculating an alexithymia level of said at least one healthy human subject, and wherein said selecting comprises selecting said at least one healthy human subject to participate in said NF resilience training based on said calculated alexithymia level.

Example 44. A method according to example 43, wherein said calculating comprises calculating an alexithymia level of said at least one healthy human subject using a Toronto Alexithymia Scale or variations thereof.

Example 45. A method according to any one of examples 42 to 44, wherein said assessing a learning model of decision making processes, comprises quantifying a learning model of decision making processes of said at least one healthy human subject, and wherein said selecting comprises selecting said at least one healthy human subject to participate in said NF-resilience training based on the results of said quantification.

Example 46. A method for selection of at least one healthy human subject to an occupation involving stress, comprising:

assessing alexithymia level and/or assessing a learning model of decision making processes of said at least one healthy human subject;

determining if said at least one healthy human subject is capable of performing EEG-NF resilience training and/or reaching a desired goal of said training, based on results of said assessing;

selecting said at least one healthy human subject to said occupation based on the results of said determining.

Example 47. A method according to claim 46, wherein said assessing alexithymia level comprises calculating an alexithymia level of said at least one healthy human subject, and wherein said determining comprises determining if said at least one healthy human subject is capable of performing said EEG-NF resilience training and/or reaching said desired goal of said training, based on said calculated alexithymia level.

Example 48. A method according to any one of examples 46 or 47, wherein said assessing a learning model of decision making processes, comprises quantifying a learning model of decision making processes of said at least one healthy human subject, and wherein said determining comprises determining if said at least one healthy human subject is capable of performing said EEG-NF resilience training and/or reaching said desired goal of said training, based on said quantified learning model.

Example 49. A resilience training system, comprising:

a user interface;

one or more electrodes configured to measure EEG signals;

a control unit electrically connected to said user interface and to said one or more electrodes, wherein said control unit is configured to:

(a) display an interface to a subject by said user interface, wherein said interface follows an activity level of at least one stress-related brain area of said subject;

(b) provide instructions using said user interface to said subject how to modulate activity of said stress-related brain regions;

(c) record EEG signals from said subject by said one or more electrodes;

(d) analyze said recorded EEG signals to identify at least one EEG signature in said recorded EEG signals indicating an activity level of said at least one stress-related brain area

(e) determine an activity level of said stress-related brain regions;

(e) modify said interface according to said determined activity.

Example 50. A system according to example 49, comprising a memory, and wherein said control unit is configured to identify said at least one EEG signature in said recorded EEG signals using at least one algorithm, a lookup table and/or at least one EEG signature or indication thereof stored in said memory.

Example 51. A system according to any one of examples 49 or 50, wherein said control unit is configured to calculate a resilience score indicating an ability of said subject to module an activity of said stress-related brain regions, based on said determined activity.

Example 52. A system according to any one of examples 49 to 51, wherein said control unit is configured to modify said instructions delivered by said user interface according to said determined activity.

Example 53. A system according to any one of examples 50 to 52, wherein said control unit is configured to display said interface and/or to provide said instructions according to an alexithymia level or indication thereof stored in said memory.

Example 54. A system according to any one of examples 50 to 52, wherein said control unit is configured to display said interface and/or to provide said instructions according to a quantified learning model of said subject or indication thereof stored in said memory.

Example 55. A resilience training system, comprising:

a user interface;

one or more electrodes configured to measure EEG signals;

a control unit electrically connected to said user interface and to said one or more electrodes, wherein said control unit is configured to:

(a) deliver one or more stress-evoking perturbations using said user interface to a human subject, wherein said perturbations are selected to affect activation of at least one deeply located limbic area using said user interface;

(b) provide instructions using said user interface to said human subject to perform one or more of mental or physical activities;

(c) record EEG signals from said human subject by said one or more electrodes;

(d) analyze said EEG signals to identify at least one EEG signature in said recorded EEG signals indicating an activity level of said at least one deeply located limbic area;

(e) determine whether the recorded EEG signals correspond with a desired activation level of said limbic areas based on said identified at least one EEG signature;

(e) deliver an indication to said human subject using said user interface based on said determining results.

Example 56. A system according to example 55, wherein said control unit is configured to modify said delivery of said stress-evoking perturbations and/or said stress related perturbations if said recorded EEG signals and/or the identified EEG signature do not correspond with a desired activation level.

Example 57. A system according to example 55, wherein said control unit is configured to modify said provided instructions if said recorded EEG signals and/or said identified EEG signature do not correspond with a desired activation level.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. For example, hardware for performing selected tasks according to some embodiments of the invention could be implemented as a mobile device, a cellular device, a wearable device or any other devices that monitor an individual. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, or a mobile device, for example a cellular phone, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as modifying an interface presented to a subject based on limbic areas, for example the amygdala activity and/or relating measured EEG signals to activation of specific brain regions, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flow chart of a general resilience training neurofeedback (NF) process, according to some exemplary embodiments of the invention;

FIG. 1B is a flow chart of a process for activation of a resilience factor, according to some exemplary embodiments of the invention;

FIG. 1C is a flow chart of an amygdala-Electrical Finger Print neurofeedback process, according to some exemplary embodiments of the invention;

FIG. 1D is a block diagram for an amygdala-Electrical Finger Print neurofeedback system, according to some exemplary embodiments of the invention;

FIG. 1E is a flow chart of a process for assessment of a subject in a timed relationship with resilience training, according to some exemplary embodiments of the invention;

FIG. 1F is a graph showing a correlation between success in a NF training and tendency for model-based learning, in a validation experiment and according to some embodiments of the invention;

FIG. 1G is a graph showing a correlation between standard deviation of EEG finger prints identified in EEG signals recorded during training and model-based learning coefficient values, in a validation experiment, and according to some embodiments of the invention;

FIGS. 2A and 2B are schematic illustrations of: (A) an experimental time-line of a NF training, and Pre-/Post-NF assessments, and (B) stages of an EEG training session as performed in an experiment and according to some embodiments of the invention;

FIGS. 3A-3E are graphs related to NF learning according to the validation experiment and according to some embodiments of the invention;

FIG. 3F is a graph showing changes in Amyg-EFP amplitude between different training sessions performed as part of the validation experiment and according to some embodiments of the invention; the Amyg-EFP amplitude was measured during a rest stage of each training session;

FIGS. 4A-4E are graphs describing outcomes of NF training per group according to a validation experiment and according to some embodiments of the invention;

FIGS. 5A-5C are graphs and a heat map image describing Amygdala-fMRI-NF, one month following Amyg-EFP-NF training, according to a validation experiment and according to some embodiments of the invention;

FIG. 6 is a schematic illustration and a heat map of the Amyg-EFP signal extraction process, according to a validation experiment and according to some embodiments of the invention;

FIGS. 7A and 7B are box plots showing the distribution of Amyg-EFP signal modulation (y-axis; Regulate vs Watch) across the six sessions (x-axis; S1-S6), according to a validation experiment and according to some embodiments of the invention;

FIGS. 8A-8D are diagrams and graphs describing NF learning of the control signal (A/T ratio) in the Control-NF group (n=38), according to a validation experiment and according to some embodiments of the invention;

FIGS. 9A and 9B are box plots describing the distribution of (A) alexithymia ratings and (B) eStroop performance before (dashed bars) and after (solid filled bars) NF training for each group [Amyg-EFP NF (red; n=88), Control-NF (blue; n=38), NoNF (grey; n=43)], according to a validation experiment and according to some embodiments of the invention;

FIG. 10 is a box plot showing the distribution of amygdala BOLD activity (y-axis; beta weights) during the Watch (pattern filled bars) and Regulate (solid filled bars) conditions according to a validation experiment and according to some embodiments of the invention: (10A) Amyg-EFP group (red, n=30). (10B) NoNF (gray; n=26). The mean and median are marked respectively by an X and a line inside each box. Whisker lines represent 1.5X interquartile range;

FIGS. 11A-11D are an illustration showing an Amygdala-fMRI-NF paradigm, according to a validation experiment and according to some embodiments of the invention: the fMRI-NF paradigm followed similar block design used during EEG-NF training, with an interface composed of a 3D animation of a character moving forward via skateboard on a road. Momentary BOLD beta weight (Regulate vs Watch) from the pre-defined right amygdala ROI was used to set the speed of the moving skateboard on the screen; and

FIG. 12 is a histogram showing the percentage of participants (y-axis) in the Amyg-EFP-NF group (n=88) that reached their best performance (minimum [Regulate vs Watch]) in each session (x-axis; S1-S6), according to a validation experiment and according to some embodiments of the invention.

### DESCRIPTION OF SPECIFIC EMBODIMENTS OF THE INVENTION

The present invention, in some embodiments thereof, relates to emotion regulation training and, more particularly, but not exclusively, to stress regulation training.

An aspect of some embodiments relates to promoting resilience by modulating activity of emotion related brain regions, for example deep-brain limbic areas. In some embodiments, the deep-brain limbic areas comprise the amygdala. In some embodiments, resilience is promoted in human healthy subjects expected to undergo a stressful experience. In some embodiments, resilience is promoted in healthy human subjects that are currently experiencing stress. As used herein, a healthy subject is a subject having a stress-indicating physiological factor, for example cortisol, within normal range of values, and/or a subject that is not diagnosed of and is not treated for a mental health disturbance, for example a mental health disturbance related to stress.

As used herein, the “resilience” of a subject refers generally to the ability of a subject to resist and/or overcome deleterious short- and or long-term effects associated with stressful stimuli, for example a stressor, by optionally, performing brain training aimed to establish self-control over at least one brain area, for example a limbic area and/or a neural network, which are markers of stress vulnerability.

According to some embodiments, healthy human subjects expected to undergo a stressful event are selected according to an occupation, for example an occupation with high probability for stress occurrence. Alternatively or additionally, healthy human subjects expected to undergo a stressful event are selected according to their geographical location, for example subjects that live in geographical locations prone to natural disasters or subjects that live in regions of geopolitical instability.

According to some embodiments, healthy human subjects currently experiencing stress are selected based on levels of a stress-related physiological parameter, for example heart rate, blood pressure, electrical conductivity of the skin, muscle tone, hormones level for example cortisone levels or any combination of the physiological parameter. Alternatively or additionally, healthy human subjects currently experiencing stress are selected based on subjective measurements, for example self-report and/or observation of an expert, for example a psychologist and/or a psychiatrist. In some embodiments, a mobile device, for example a cellular phone is used for objective-camera viewing the person face/gestures/pupils at home or work, and/or using wearables physiological sensors that can monitor the physiological parameter, for example heart rate sensors or sleep pattern, EMG sensors and/or skin conduction rate (SCR) sensors.

According to some embodiments, at least one subject, for example at least one healthy human subject, is selected based on a type of leaning, selectivity to placebo, type of emotion regulation, for example suppression or appraisal, and/or whether the at least one subject suffers from physiological responses associated with stress, for example sleeping disturbances.

According to some embodiments, one or more subjects, for example healthy human subjects, are selected based on results of an assessment performed prior to the resilience training, for example prior to EEG-NF. In some embodiments, the assessment comprises an assessment of an alexithymia level of the subject, for example to determine an alexithymia baseline level. In some embodiments, the ability of the subject to participate in the resilience training and/or to reach a desired goal of the training, for example a desired activity level of one or more stress-related brain regions or a desired resilience score, is predicted based on the results of the alexithymia assessment. Alternatively or additionally, the assessment comprises assessment of a learning model of decision-making processes of the subject. Optionally, one or both of the alexithymia level and/or learning model are assessed during the resilience training, for example during the EEG-NF. Additionally or alternatively, the assessment comprises performing one or more stress tests, for example the Trier Social Stress Test (TSST), the Montreal Imaging Stress Task (MIST), threat of obtaining painful stimuli, horror movie or virtual reality stressful scenario. In some embodiments, a subject is selected for a resilience training according to a subject selectivity to placebo, an emotion regulation capability, for example suppression versus appraisal of the subject, personality trait (e.g. neuroticism), anxious tendency, learning style, cognitive flexibility. Alternatively or additionally, the subject, for example a healthy human subject is selected for the NF training based on physiological or anatomical parameters associated with a higher probability to develop post-traumatic stress disorder (PTSD) in response to a stressor in the future, for example a small hippocampus.

According to some embodiments, at least one parameter of the resilience training, for example overall duration of the training, number of training sessions, interval between training sessions, and/or time duration in which a subject tries to regulate the activity of one or more stress-related brain regions, is modified based on the assessment results.

Herein, the “resilience” of a subject refers generally to the ability of a subject to resist and/or overcome deleterious short- and or long-term effects associated with stressful stimuli, for example a stressor, for example avoidance behavior, violence, anger bursts, productivity reduction, cognitive difficulties, reduced mood, disturbed sleep, high arousability, and/or dysregulated mood. Stress resilience includes resilience to acute stress caused for example by application of a stressor for a short time period, for example up to 1 hour, up to 30 minutes, up to 10 minutes or any intermediate, shorter or longer time period. Additionally, stress resilience includes resilience to chronic stress, for example chronic stress caused by application of a stressor for time periods equal or longer than 1 hour, for example longer than 2 hours, longer than 10 hours, longer than 1 day or any intermediate, shorter or longer time periods.

According to some embodiments, a level and/or pattern of subject brain activity in certain regions related to response to stress, for example the amygdala or other brain regions of the limbic system, under a condition of stressful stimulation is understood, herein, to be itself a metric of the subject's resilience. Without being bound to any theory, the amygdala, for example, was shown to play a major role in the processing of physiologic and behavioral response to stress, often reflected in hyper activation that could also be a predisposing factor for stress vulnerability. Thus, for example, a subject's resilience is considered to have been “promoted” when amygdala activity or activity of other brain regions related to response to stress, for example one or more brain regions of the limbic system, is reduced in a test condition for a certain subject compared to amygdala activity in a baseline and/or control condition.

According to some embodiments, resilience is promoted by training a subject, for example a trainee, how to modulate activity of one or more stress-related brain areas, for example brain areas of the limbic system. In some embodiments, the brain areas of the limbic system comprise the amygdala. In some embodiments, the resilience is promoted by training a subject to downregulate the one or more stress-related brain areas, for example amygdala. Alternatively or additionally, resilience is promoted by training a subject to upregulate activation of other brain regions, for example the ventro medial prefrontal cortex. Optionally, resilience is promoted by training a subject how to modulate or regulate activation of neural circuits related to stress regulation and/or resilience promotion. Optionally, resilience is promoted by training a subject to modulate a dynamic function of a system that determines a resilience level, for example a system that generates a resilience score.

According to some exemplary embodiments, a selective activation of the one or more brain regions is monitored using recorded EEG signals. In some embodiments, the EEG signals are recorded before the resilience training, for example as part of assessment stage, during and/or following the resilience training. In some embodiments, a specific EEG signature, for example an EEG-fingerprint, indicating a selective activity of the one or more brain regions is identified in the recorded EEG signals.

According to some embodiments, resilience is promoted by performing exercises, for example mental and/or physical and/or neural exercises, as part of the resilience training, for example in the EEG-NF. In some embodiments, the exercises are personalized and optionally change with time and context. In some embodiments, the exercises downregulate activity of the at least one stress-related brain regions, for example the amygdala activity. Alternatively or additionally, the exercises upregulate other brain regions, for example the medial prefrontal cortex. Optionally, the exercises modulate a combination of regions related to one or more of salience, executive functions or mentalization networks. In some embodiments, the exercises modulate a specific network metric such as influence of one or more nodes in a network or graph connectivity of one or group of nodes. In some embodiments, the exercises regulate limbic-prefrontal connectivity and/or context specific EEG markers.

According to some embodiments, the exercises, for example the mental and/or physical and/or neural exercises, comprise exercises which affect alpha/theta ratio. In some embodiments, the exercises comprise guiding eye movement with a dot, for example as performed in EMDR. Alternatively or additionally, the exercises comprise biofeedback guided by at least one physiological parameter, for example heart rate, blood pressure and/or skin conductance response (SCR).

According to some exemplary embodiments, a resilience promoting activation (also termed herein as resilience factor, for example a resilience neural factor (RNF)), comprises execution of a neurofeedback (NF) training protocol with or without trainee active volition in modifying brain function. In some embodiments, in the NF training protocol the trainee receives a human detectable indication, for example a feedback. In some embodiments, the feedback is continuously delivered to a subject or incrementally delivered to a subject. In some embodiments, the feedback follows at least one parameter related to the amygdala or other brain region or a neural network, for example activity level, connectivity level, correlation level, function level, influence level for example a partial correlation effect, cohesion level for example temporal modulation similarity and/or distribution pattern similarity. In some embodiments, the feedback follows an activity level of the trainee brain function, for example the function of the amygdala or other stress-related brain regions with or without awareness of the trainee. Alternatively, the feedback follows a modulation level of the activity of one or more stress-related brain areas, for example an amygdala activity modulation level of the trainee amygdala with or without self-awareness.

According to some embodiments, the feedback follows at least one parameter related to recovery from stress, for example recovery dynamics, recovery duration, and/or recovery process, for example as indicated by subjective self report, objective physiological measures, for example heart rate or pupil dilation and/or neural indicator, for example increased activation in salience system, and/or epigenomic measures. In some embodiments, the recovery from stress is measured by identifying changes in one or more signatures, for example an EEG signature in recorded EEG signals, indicating an activity level or changes in an activity level of one or more stress-related brain regions.

Here an activity level is used as an example for a parameter related to the amygdala or other brain regions or neural network. However it should be understood that any other parameter relating to a brain area, for example connectivity level, correlation level, function level, influence level for example a partial correlation effect, cohesion level for example temporal modulation similarity and/or distribution pattern similarity, can be used with the methods, devices and systems described in this application instead of activity level.

According to some embodiments, the NF training protocol comprises a covert NF training protocol which is executed without trainee volition. In some embodiments, the covert NF training protocol comprises covert monitoring and/or covert feedback, for example covert reward. Alternatively, the reward is openly displayed and is not covert feedback. Optionally, the covert feedback is personalized to a specific trainee.

According to some embodiments, an interface, for example a multi-media interface is presented to the trainee (subject) during the training program. In some embodiments, the interface comprises one or more of a scenario, optionally a fixed scenario, for example a multimodal-audio-visual scenario, an audio-somatic scenario, a visual-somatic-audio scenario, a continuous scenario, and/or a gamified (optionally goal-directed) scenario. Additionally or alternatively, the interface comprises a scenario developed in virtual or augmented reality, an intermittent feedback related to an exciting/stressing occurrence for example a car race, a medical procedure, interaction between two or more subjects, optionally with an outcome, for example an outcome related to brain modulation. Optionally, the outcome is delivered every few seconds or minutes. In some embodiments, the interface comprises visual and/or audio signals.

According to some embodiments, the feedback is delivered to the subject by changing at least one parameter related to the presented interface and/or changing a behavior of one or more avatars in a group. In some embodiments, the trainee identifies which avatar is most critical for the feedback. In some embodiments, the at least one interface parameter comprises one or more of number, shape size, color or sound of presented objects in the interface. Alternatively or additionally, the at least one interface parameter comprises interaction between objects and/or sounds generated by one or more of the objects. In some embodiments, the interface comprises goal directed behavior which optionally is configured to affect modulation of a selected brain-target.

According to some embodiments, the interface is personalized for a selected subject or to a group of subjects. Optionally, the interface is personalized according to the subject profession, life memories, and/or life experience; for example positive or negative memories or experience. In some embodiments, the interface comprises one or more stressors configured to induce a stress response or positive feeling in a subject, for example by delivery of likable music.

According to some exemplary embodiments, the amygdala activity level is determined based on measurements of at least one physiological parameter, for example based on EEG signals and/or fMRI measurements. In some embodiments, a relation between the measured physiological parameter and a fingerprint of an activity or activity modulation of one or more brain regions, for example the amygdala activity or a modulation of amygdala activity is determined. In some embodiments, the measured physiological parameter comprises EEG signals and the fingerprint comprises an Amygdala-Electrical Finger Print (Amyg-EFP). In some embodiments, the feedback delivered to the user reflects the amygdala activity and/or changes in the amygdala activity. Alternatively or additionally, the feedback reflects connectivity to other regions, for example ventral striatum, medial PFC, Inferior Frontal Gyms, Insula or ACC, or any combination of the regions. In some embodiments, the delivered feedback is based on the measured EEG signals and on the relation between the measured EEG signals and the fingerprint. Alternatively or additionally, the delivered feedback is based on a relation between the fingerprint and other stress markers, for example pupil dilation, heart rate, blood pressure and/or skin conductance response.

According to some embodiments, the NF training protocol comprises a reinforcement learning procedure that is optionally interfaced by multimodal agitating a 2D or a 3D scenario. In some embodiments, the momentary scenario agitation corresponds to the trainees' amygdala activity modulation that is represented by fMRI-inspired EEG model; termed Amygdala-Electrical

Finger Print (Amyg-EFP).

According to some embodiments, the NF protocol is a protocol of one or more training sessions, for example 2, 4, 5, 6 sessions or any intermediate smaller or larger number of sessions. In some embodiments, the one or more training sessions are applied anywhere, optionally at any-time, without the need for a special relaxing context of a quiet room or eyes-closed. In some embodiments, the NF protocol is performed in a multi-modal noisy/stressful context, for example with eyes-opened, which optionally enables a translation of the NF protocol to on-going daily situations. According to some embodiments, the trainees participate in a scenario, for example a game-like situation while exploring their mentalization sets that correspond to Amyg-EFP down- or up modulation. In some embodiments, the scenario is part of an interface between the trainee and one or more objects, for example virtual objects optionally presented on a display. In some embodiments, the trainees are subjects undergoing a stressful life period, for example soldiers in a military training, flying cadets, fire fighters, or early responders to emergency events. In some embodiments, the trainees learnt within a period of 1-10 sessions, for example 1-5 sessions, 3-6 sessions, 4-8 sessions or any smaller or larger number of sessions, how to associate their Amyg-EFP signal modulation with a specific mentalization. In some embodiments, the specific metallization is individually or given by instructions. Optionally, the trainees are able to apply the learned resilience skill outside the training context, for example without feedback.

According to some embodiments, a NF training protocol using the Amyg-EFP (Amyg-EFP-NF) provides an adaptive skill to better cope and fit with life adversities. In some embodiments, subjects undergoing training reduce Alexithymia. As used herein. Alexithymia means an inability to define and appraise emotional feelings in self and others. In some embodiments, reducing Alexithymia is as an active, dynamic adaptation process when facing stress, for example a resilience factor activating process. In some embodiments, activation of resilience factors by the training program changes performance on emotional conflict task (known as the emotional stroop). In some embodiments, improved speed of responding to emotional conflicts indicates enhanced emotion regulation that is optionally automatically employed.

According to some exemplary embodiments, resilience factor activation corresponded to changes in the amygdala with training. In some embodiments, increased down regulation of

Amygd-EFP signal during NF sessions correlates with more decreased Alexithymia or with a decrease in alexithymia levels. In some embodiments, operating a resilience factor activates, for example, a functional negative feedback system in response to stressful challenges in the environment. Optionally, activation of the functional negative system induces an internal resilience process. In some embodiments, internal resilience process is recruited outside of the training context.

According to some exemplary embodiments, subjects undergoing Amyg-EFP-NF training modulate amygdala BOLD signal during fMRI-NF while co-activating medial prefrontal cortex. In some embodiments, the medial PFC is a core region in activating emotion regulation processes in humans. Alternatively or additionally, subjects undergoing Amyg-EFP-NF training modulate a relation between posterior and anterior insula, for example downregulate posterior insula and upregulate anterior insula.

According to some embodiments, the interface is a generic interface. Alternatively, the interface is a personalized interface. In some embodiments, the personalized interface comprises one or more personalized stress related scenarios. In some embodiments, the personalized interface comprises stressors, for example in the one or more scenarios, known to induce stress in a selected human subject. Optionally, the stressors relate to the human subject profession. In some embodiments, the stressors relate to interactions with other humans, optionally presented as avatars.

According to some embodiments, the NF is a process-based NF, for example, if the subject is a soldier, then a personalized scenario comprises a battle, a check-point or any other scenario related to the soldier. In some embodiments, for example, if the subject is a fire-fighter, then the personalized scenario comprises firefighting, for example in a house. In some embodiments, if the subject is a policeman then the personalized scenario comprises protesting civilians or gun-shooting in a crowd.

According to some embodiments, an interface configured to induce stress in a subject modulates towards a less-stressful interface following a desired activity and/or a desired activity modulation of the brain region, for the amygdala.

According to some exemplary embodiments, the personalized interface is configured to simulate potential daily stressors. In some embodiments, the interface includes an interaction, for example an interaction between an avatar of the subject and other avatars displayed on a screen, for example using an outside-in approach, opposed to a first person view or an immersive approach, for example in an outside-in approach a virtual avatar of a subject negotiates virtual objects in an environment. Alternatively, the interface includes a scenario displayed on a screen, optionally with one or more virtual objects, for example virtual avatars, and the subjects negotiate the virtual objects using an outside-in approach. Optionally, the subjects interact the virtual subjects by verbal interaction. In some embodiments, the interface comprises an augmented reality environment, for example an environment which displays one or more virtual objects in a real world environment presented on a display or an environment which displays brain activity of a first person to a second person.

An aspect of some embodiments relates to promoting resilience to stress by modulating activation of deep-brain areas related to emotion control, for example deep brain limbic area, the amygdala, locus coeruleus, pulvinar and/or cortical control areas such as medial prefrontal cortex, inferior frontal gyms or insula or their combination. In some embodiments, the activation modulation of the emotion related deep brain regions is achieved by applying NF, for example EEG-based NF or a NF process based on measurement of at least one electrophysiological parameter. Optionally, the at least one electrophysiological parameter comprises a stress-related electrophysiological parameter, for example a stress-related electrophysiological parameter as inspired by fMRI or peripheral stress markers.

As used herein, the term stress resilience means a dynamic neuropsychological process which refers to the maintenance of mental health despite exposure to psychological or physical adversities. It is assumed to be a protective mechanism against stress that prevents the consequence development of psychopathologies. Resilience (in opposition to vulnerability), focuses on a dynamic process of effective adaptation back to baseline when homeostasis is disturbed [1].

According to some embodiments, the interface is a generic interface. Alternatively, the interface is a personalized interface. In some embodiments, the personalized interface comprises one or more personalized stress related scenarios. In some embodiments, the personalized interface comprises stressors, for example in the one or more scenarios, known to induce stress in a selected human subject. Optionally, the stressors relate to the human subject's profession.

According to some embodiments, for example, if the subject is a soldier, then a personalized scenario comprises a battle, a check-point or any other scenario related to the soldier. In some embodiments, for example, if the subject is a fire-fighter, then the personalized scenario comprises fire breaking, for example in a house. In some embodiments, if the subject is a policeman then the personalized scenario comprises protesting civilians or a gun shooting in a crowd.

According to some embodiments, the personalized interface is configured to provoke potential daily stressors. In some embodiments, the interface includes an interaction, for example an interaction between an avatar of the subject and other avatars displayed on a screen, for example using an outside in approach when a virtual avatar of a subject negotiates virtual objects in an environment. Alternatively, the interface includes a scenario displayed on a screen, optionally with one or more virtual objects, for example virtual avatars, and the subjects negotiate the virtual objects using an outside-in approach. In some embodiments, the interface comprises an augmented reality environment, for example an environment which displays one or more virtual objects in a real world environment presented on a display (i.e. augmented reality).

According to some embodiments, the applied NF comprises an implicit, for example a covert-NF training. In some embodiments, in the implicit training rewards are provided in contingency to resilience factor modulation. For example, one or more subjects will be participating in a scenario series game in which they experience a situation and while interacting with elements in the environment, predetermined rewards are provided. In some embodiments, the rewards are provided only if the negotiation of the one or more subjects within the situation is mediated by a resilient neural factor, for example amygdala down regulation or medial prefrontal cortex up regulation.

An aspect of some embodiments relates to modifying at least one parameter related to activity and/or function of one or more stress-related brain regions to bring a subject to within a desired range of stress levels. In some embodiments, stress-related brain regions are brain regions which mediate emotional responses to experiences of stressors. In some embodiments, values of the at least one modified parameter indicate an activity level of the one or more stress-related brain regions. Optionally, measuring values of the at least one modified parameter allows, for example, to evaluate, optionally quantitatively, an activity level of the one or more stress-related brain regions.

According to some exemplary embodiments, the activity and/or function of the one or more stress-related brain regions is modified using neurofeedback (NF), for example electrical fingerprint neurofeedback. In some embodiments, two or more stress-related brain regions are modulated. In some embodiments, at least one of the two or more stress-related brain regions is down regulated, and optionally, at least one of the two or more stress-related brain regions is upregulated. In some embodiments, activity and/or function of the one or more stress-related brain regions is upregulated and/or downregulated to reach the desired target.

According to some exemplary embodiments, the desired range of stress levels is personalized for a subject. In some embodiments, the desired range of stress levels is determined according to an occupation of the subject.

An aspect of some embodiments relates to assessing resilience of a human subject by monitoring activity modulation and/or activity state of brain regions related to emotion control, for example deep-brain limbic areas. In some embodiments, the activity modulation and/or activity state of the brain regions is monitored in response to stress-evoking provocations. In some embodiments, the deep-brain limbic areas comprise the amygdala. In some embodiments, activity modulation and/or activity state of the brain regions are monitored based on a relation between measured EEG signals and one or more Electrical Finger Prints.

According to some embodiments, resilience is assessed in a subject by monitoring changes in amygdala activity in response to a controlled stress induction. In some embodiments, resilience is assessed by monitoring an increase in amygdala or other stress related area activity following a controlled stress induction. In some embodiments, resilience is assessed by monitoring a time duration that passes until reaching amygdala activity baseline levels, for example baseline levels prior to stress induction. In some embodiments, the NF training protocol is configured to improve at least one parameter related to stress recovery, for example recovery dynamics or a recovery process. In some embodiments, resilience factor activation comprises activation of mechanisms that improve recovery from stress.

An aspect of some embodiments relates to performing a NF training protocol for promoting resilience in a stressed environment, for example an environment comprising a stressor. In some embodiments, a subject undergoing the NF training protocol is located in a stressed environment. In some embodiments, the NF training protocol is delivered using a single EEG electrode and optionally lasts a short time period of less than 30 minutes for each training session, for example less than 20 minutes, less than 15 minutes, less than 10 minutes, less than 5 minutes or any intermediate, shorter or longer time period. In some embodiments, the interface presented to the trainees includes a virtual non-stressed environment, and was optionally presented as a gamified environment.

A possible advantage of delivering a NF training protocol with a virtual non-stressed environment is that it increases the motivation of the stressed subject to perform the NF training protocol and/or to activate specific mental to brain processes.

According to some embodiments, instead of using a general anatomical marker for NF training, it is possible to use a marker of modulation in neural processing in response to a well-defined stressor, for example an emotion provoking stimulus. In some embodiments, the neural processing modulation marker is generated, for example, by correlating fMRI and EEG signals during stress induction. Alternatively, the neural processing modulation marker is generated, for example, by obtaining a longitudinal densely sampled measurements of amygdala EFP along with objective and subjective indices of stress, for example heart rate variability and subjective report, respectively. In some embodiments, the neural processing modulation marker comprises a multivariate neural signature, for example a decoder that optionally predicts individual stressful state and can be used for personalized neural target for NF training.

According to some embodiments, the NF procedure is combined additional resilience factor activation procedures which are optionally not neutrally based, for example reappraisal training or improving response to emotional distractors optionally via emotional stroop or attention threat bias modification or Eye Movement Desensitization and Reprocessing (EMDR). A potential advantage of combining two or more resilience factor activation procedures is that it enables synergistic impact of coping with stress.

The burden of stress on subjects health and well-being has been known for decades. More than half a billion people around the world suffer from stress related psychopathology annually. The stressors could be endogenous or exogenous, including traumatic events, daily demands and hurdles, family ordeals and mishaps and physical illnesses, leading to annual economic cost of 200 billion euro [1]. It has largely agreed that this epidemiology is due to both lack of treatment effectiveness as well as prevention efforts.

According to some embodiments, a ‘prevention gap’ is overcome by focusing on promoting resilience to stress through mental-fitness training rather than reducing its disease related burden. It does so, by providing a way for brain training aimed to establish self-control over the amygdala; a known marker of stress vulnerability with respect to real life stressors [2,3,4].

According to some embodiments, the training of the amygdala takes place through NeuroFeedback (NF), which is for example a reinforcement learning procedure that is optionally interfaced by multimodal agitating 3D scenario. In some embodiments, the momentary scenario agitation corresponds to the trainees' amygdala activity modulation that is represented by fMRI-inspired EEG model; termed Amygdala-Electrical Finger Print (Amyg-EFP). In addition, a one-class Amyg-EFP model developed on one group, can capture ongoing modulation of amygdala fMRI activation in another group [5].

According to some embodiments, to empower stress resilience, a short NF protocol of six sessions is provided. In some embodiments, the training protocol is applied anywhere at any time, without the need for a special relaxing context of a quiet room or eyes-closed. In some embodiments, the training method is done within a multi-modal noisy/stressful context with eyes-opened, which optionally enables its translation to on-going daily situations. In some embodiments, the trainees participate in a game-like situation while exploring their mentalization sets that correspond to Amyg-EFP down- or up modulation.

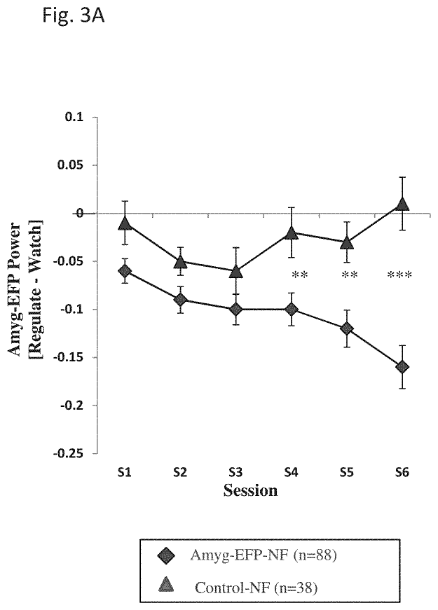

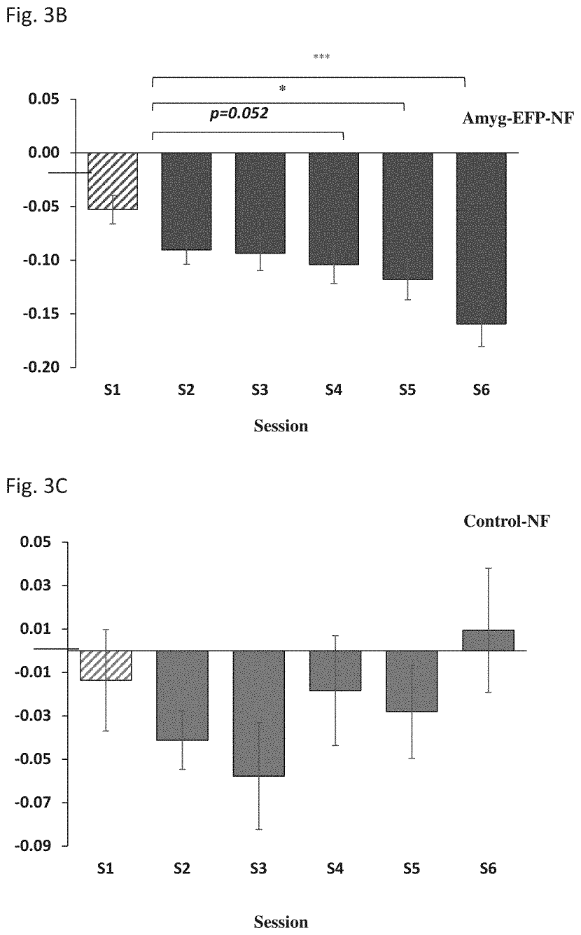

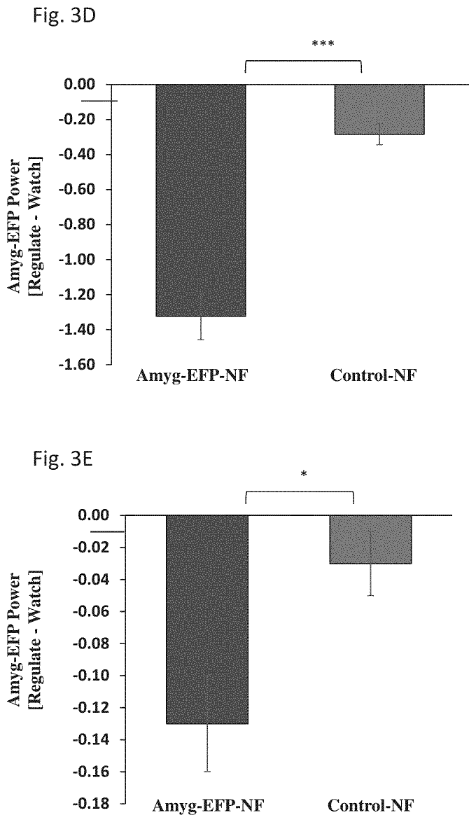

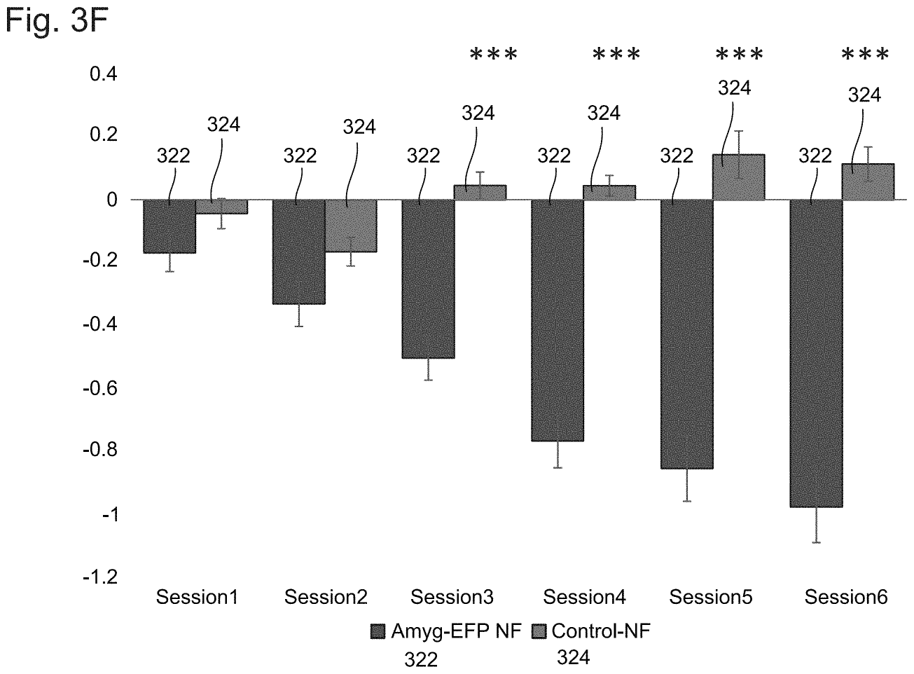

In a validation experiment described in this application, a large scale study of 180 young soldiers undergoing a stressful life period in their military training was conducted. The results show that more than 90% of the subjects learnt within 3-6 sessions how to associate their Amyg-EFP signal modulation with a specific mentalization, and were able to later apply the new resilience skill outside the training context; i.e. without feedback (also known as “transfer trial”), for example as shown in FIGS. 3A-3D.

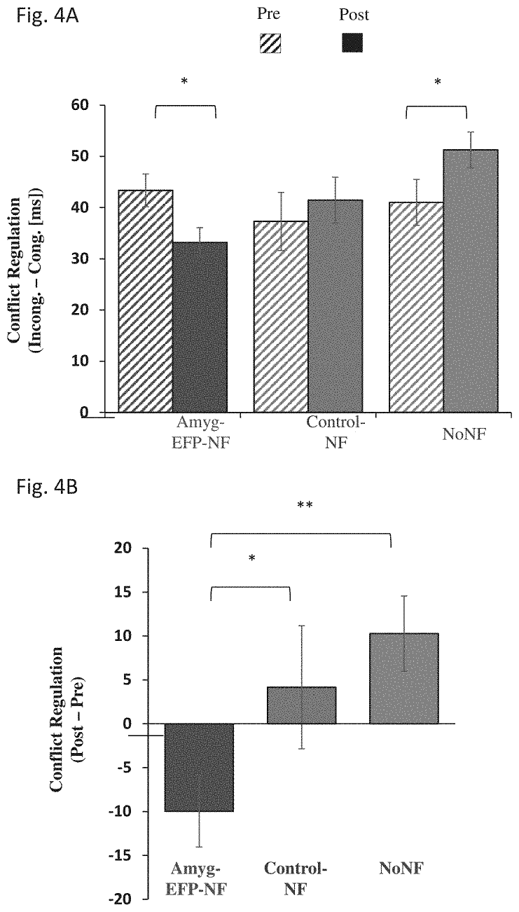

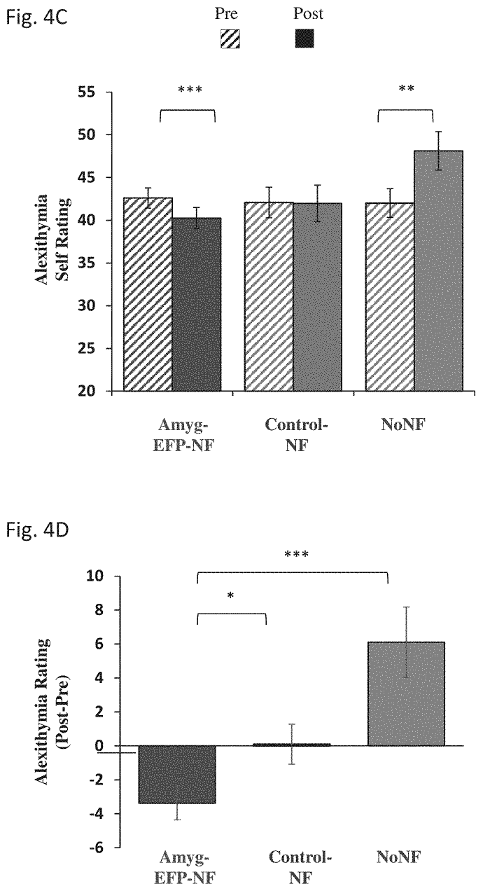

In some embodiments, Amygd-EFP-NF is used as a procedure for activating a resilience process, as it provides an adaptive skill to better cope and fit with life adversities. The validation experiment results described that the ones undergoing training decreased their Alexithymia measure while the ones not doing training during the same stressful period actually increased it, for example as shown in FIGS. 4C-4D.

As used herein, Alexithymia literarily stands for “no words for emotions”; an inability to define and appraise emotional feelings in self and others. Hence, reducing Alexithymia is regarded as an active, dynamic adaptation process when facing stress thus; in other words a resilience factor [1]. Further, examples provided herein demonstrate that the NF training used for activation of resilience is correlated with a change in performance on emotional conflict task (known as the emotional stroop). Improved speed of responding to emotional conflicts indicates enhanced emotion regulation that is automatically employed, for example as shown in FIGS. 3A-3B.

The validation experiments showed that the observed improvement in behavioral indices of resilience activation corresponded to changes in the amygdala with training. The results indicate that greater down regulation of Amygd-EFP signal during NF sessions is correlated with more decreased Alexithymia, in the test group compared to the control group, for example as shown in FIG. 4E. In some embodiments, reduction in alexithymia levels compared to an alexithymia level base line and/or to previously measured alexithymia levels is used to monitor the NF training. In some embodiments, a pre-determined level of alexithymia is used as an end point of the NF training or a desired goal of the NF training.

The result showing that a greater down regulation of Amygd-EFP signal during NF sessions correlate with more decreased Alexithymia correspond to animal model showing that particular changes in neural activation with stress is evident in animals with greater recovery from the exposure [6,7]. In a similar manner, examples presented herein support a causal relation between successfully operating a neural representation of resilience factor and superior coping (i.s. developing less alexithymia) while dealing with military stress. The Examples presented herein also demonstrate that resilience reflects an outcome of active skill training with a real life stressful event, rather than a given trait/personality tendency.

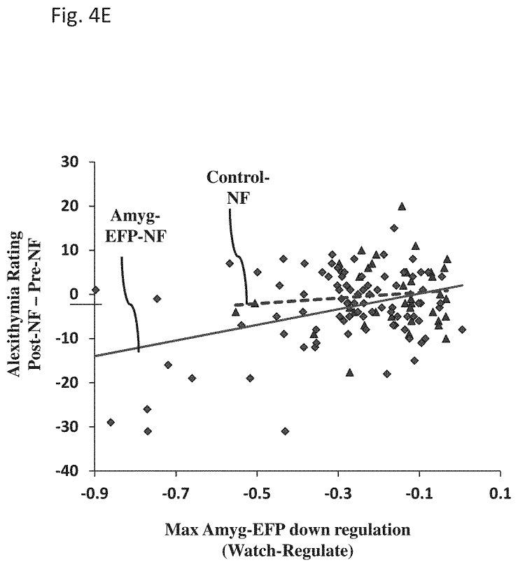

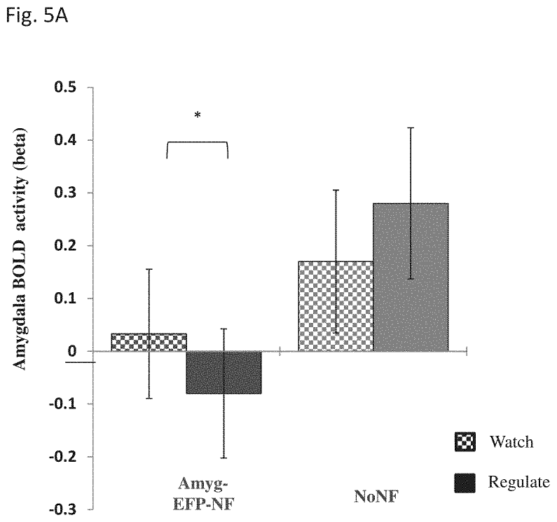

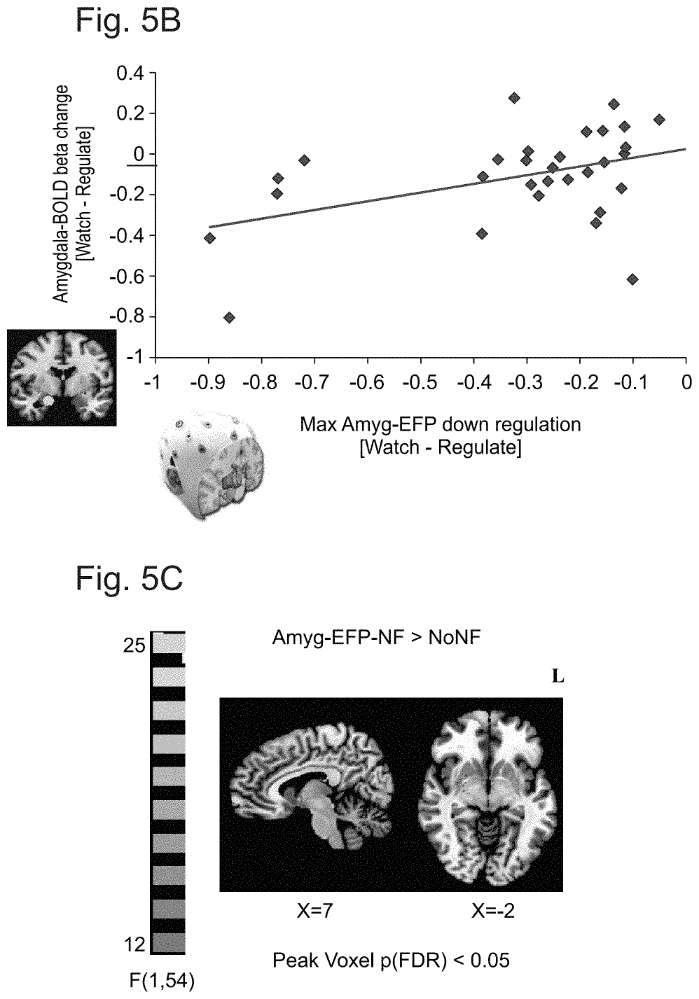

According to some embodiments, a platform for operating a resilience factor that activates a functional negative feedback system in response to stressful challenges in the environment hence, optionally inducing/igniting an internal resilience process is described. Results obtained from fMRI study performed on part of the soldiers about two months following the NF training demonstrate a long range effect of the NF training. The validation experiment results show that a group undergoing Amyg-EFP-NF training in comparison to a control group, was better in modulating their amygdala BOLD signal during fMRI-NF while co-activating their medial prefrontal cortex (FIGS. 5A-5C). The medial PFC is apparently a core region in activating emotion regulation processes in humans [8]. This later validation of fitness target engagement, corresponded to larger NF effect as indicated by lower Amyg-EFP signal achieved during training, as shown for example in FIG. 5B.

An aspect of some embodiments relates to monitoring an effect of the resilience training by measuring activity of one or more brain regions during rest stages of the resilience training. In some embodiments, changes in brain activity are measured between two or more consecutive rest stages of the resilience training. In some embodiments, during a rest stage of the training protocol, a subject is passively watching an object or a scenario, for example a scenario presented using a display. In some embodiments, during a rest stage, the subject is passive, for example the subject is not encouraged or asked to perform a cognitive or a mental task related to the presented object or to the presented scenario. In some embodiments, the measured activity of one or more brain regions during rest stages of the resilience training is used to personalize the resilience training to a specific subject, and/or to decide whether a specific subject is responsive enough to continue with the training protocol.

According to some embodiments, regulation, for example downregulation or upregulation of the activity of the one or more brain regions during rest stages as the training proceeds is indicative of success in the training. In some embodiments, the activity regulation is measured by comparison of brain activity measurements recorded during a rest stage to brain activity measurements measured in a previous rest stage. In some embodiments, a training success score is calculated based on a level of the brain activity regulation measured during consecutive rest stages. In some embodiments, a larger measured activity regulation, for example activity down regulation or activity upregulation during rest stages is indicative of a larger probability of a subject to succeed in the resilience training, for example to become more resilient to stress following the training.

According to some embodiments, at least one parameter of the resilience training is modified according to the brain activity downregulation measured during rest stages of the training. In some embodiments, the at least one parameter comprises number of training session, for example a subject showing a large downregulation of one or more brain regions during rest stages will receive less training session compared to other subjects showing smaller downregulation. In some embodiments, the at least one parameter comprises one or more of a duration of each training session and/or duration of stages in a training session, difficulty level of a task during a regulate stage and/or any parameter or parameter values related to resilience factor activation.

According to some embodiments, the calculated change in downregulation of one or more selected brain regions is compared to one or more stored values or indications thereof. Alternatively or additionally, the calculated change in downregulation is used as an input to at least one stored lookup table or at least one stored algorithm. In some embodiments, the resilience training is modified based on the results of the comparison to the one or more stored values or indications thereof and/or based on the stored lookup table or the at least one stored algorithm.

An aspect of some embodiments relates to modifying at least one parameter of a resilience training based on assessment of a trainee during the training. In some embodiments, alexithymia level measurements and/or changes in alexithymia levels during the training are used to modify at least one parameter of the resilience training. Alternatively or additionally, the activity levels or changes in the activity levels of one or more brain regions during rest stages of a training session are used to modify at least one parameter or values thereof of the training.

According to some embodiments, the at least one modified parameter comprise the number of training sessions in a complete training plan, for example the overall number of training sessions needed to reach a desired level of brain activity or a desired level of resilience to stress. Alternatively or additionally, the at least one modified parameter comprises a complexity level of a training session, for example a complexity level of a cognitive task or a scenario complexity level in one or more training sessions. Alternatively or additionally, the at least one modified parameter comprises a rate of a planned change in the complexity level of the training between two or more consecutive training sessions. Alternatively or additionally, the at least one modified parameter comprises one or more of rate of reward presentation that can be continuous, intermittent or delayed, a threshold for feedback that is optionally based on rate of learning, online calculating how well the subject is learning and modify the protocol, number of sessions based on intermittent outcome, length of NF epochs, and type and context of the feedback interface.

According to some embodiments, the alexithymia level is assessed, for example based on measurements performed prior to a training session, during a training session or after a training session. Alternatively or additionally, the alexithymia levels are measured between training sessions, for example when the trainee is not in a training facility and/or after at least 15 minutes, for example after 30 minutes, after 1 hour, after 2 hours, after a day or any intermediate, shorter or longer time period from finishing a training session. In some embodiments, alexithymia levels are measured, for example based on an interview with an expert, for example a physician and/or based on scores of a test or a questionnaire, for example scores on the Toronto Alexithymia scale (TAS-20) questionnaire.

According to some embodiments, an activity level of one or more brain regions is measured at rest stages of a training session using at least one electrode, for example an EEG electrode, measuring brain activity. Alternatively or additionally, the activity level of the one or more brain regions is measured at rest stages of a training session using MRI, for example functional MRI. In some embodiments, the activity level of one or more brain regions is measured between training sessions, for example when the trainee is not actively performing a task intended to regulate or change a presented scenario or any other condition perceived by the trainee.

An aspect of some embodiments relates to assessment of a subject prior to resilience training, for example to anticipate a success of the subject in participating in the training, and/or in reaching a desired training goal. In some embodiments, an assessment of a subject prior to resilience training is used to select a resilience training protocol or to modify an existing protocol to fit one or more characteristics of the subject, for example learning ability of the subject. In some embodiments, selecting a training protocol or modifying an existing protocol comprises using a resilience training protocol in combination with a different treatment, for example in combination with a pharmaceutical or any bio-active compound. In some embodiments, the assessment of the subject comprises measuring an alexithymia level of the subject prior to training. Alternatively or additionally, the assessment of the subject comprises quantifying learning models of the subject, for example quantification of reinforcement learning model based or model free tendency. In some embodiments, at least one parameter of the resilience training, for example the EEG-NF is modified based on the assessment.

According to some embodiments, the alexithymia levels are measured prior to training, for example based on an interview with an expert, for example a physician and/or based on scores of a test or a questionnaire, for example scores on the Toronto Alexithymia scale (TAS-20) questionnaire. In some embodiments, if the alexithymia levels are lower than a predetermined value, then the subject is excluded from the resilience training. In some embodiments, a resilience training protocol is selected for the subject based on the alexithymia measurements, for example a resilience training protocol with a baseline, for example a starting level which is adjusted to the subject. Alternatively, one or more parameters or values thereof of an existing training protocol are adjusted according to the alexithymia measurements, for example a starting level of the training, changes in the training protocol complexity between or during training sessions, number of training sessions in a training protocol, duration of each training session and/or duration of at least one stage of a treatment session.

According to some embodiments, quantifying learning models of the subject is performed, for example using a two-step task, for example as describe in Daw et al. 2011. In some embodiments, for example in the two-step task, learning models are quantified by learning coefficients of model based and model free decision making processes. In some embodiments, a resilience training protocol is selected for the subject based on the quantification of the learning models, for example a resilience training protocol with a baseline, that is adjusted to the learning model of the subject. Alternatively, one or more parameters or values thereof of an existing training protocol are adjusted according to the learning model of the subject, for example a starting level of the training, changes in the training protocol complexity between or during training sessions, number of training sessions in a training protocol, duration of each training session and/or duration of at least one stage of a treatment session.

An aspect of some embodiments relates to selecting a subject for a stressful profession, for example an occupation that involves exposure to stress, based on the ability of the subject to undergo a resilience training. In some embodiments, the subject is selected for a stressful profession based on assessment of alexithymia level of the subject. Alternatively or additionally, the subject is selected for a stressful profession based on a learning model characteristics of the subject.

According to some embodiments, an assessment of an alexithymia level and/or of a learning model of decision making processes is performed as part of a recruitment process of the subject to an occupation involving stress, for example exposure to at least one stressor in an occurrence which is higher compared to other occupations, and/or exposure to at least on stressor that causes a prolonged stress effect or that can lead to development of chronic stress. In some embodiments, a capability of a subject to perform a NF-training, for example, the resilience training, and/or to reach a desired outcome or goal of the training, is determined based on the results of the assessment. In some embodiments, the subject is selected to the stressful occupation according to the determined capability of the subject to perform the NF-training or to reach the desired outcome or goal of the training. In some embodiments, the subject is selected to the occupation based on the results of the NF training, as determined during the training and/or at the end of the training. In some embodiments, the results of the training ae determined based on the recorded EEG signals and/or based on assessments of the alexithymia level and/or of a learning model of decision making processes performed during and/or at the end of the training.

According to some embodiments, the NF-training session described herein comprises at least one training session or two or more training sessions, for example 1, 2, 4, 6, 10, 12, 20 or any intermediate, smaller or larger training sessions. In some embodiments, each training session comprises a stressor exposure stage, for example a “watch” stage as described herein. In some embodiments, in the stressor exposure stage a subject, for example a healthy human subject, is exposed to at least one stressor. In some embodiments, the at least one stressor is selected to induce a stress response, for example, a measurable stress response in the subject.

According to some embodiments, the training session comprises a stressor regulating stage, for example a “regulate” stage as described herein. In some embodiments, in a stressor regulating stage, the subject performs at least one activity, for example a mental activity and/or a physical activity, configured to regulate the stress response generated by the exposure to the stressor. In some embodiments, the at least one activity is configured to regulate, for example upregulate or downregulate an activity level of at least one brain area related to stress, for example a brain area of the limbic system. In some embodiments, a feedback is delivered to the subject according to the activity level of the at least one brain area and/or according to the level of the measurable stress response caused by the at least one stressor.

According to some exemplary embodiments, a time duration of each training session is in a range of 1 minute to 120 minutes, for example 1-30 minutes, 20-60 minutes, 40-80 minutes or any intermediate, shorter or longer time duration. In some embodiments, a time period between two consecutive training sessions is in a range of 6 hours to 1 month, for example 6 hours to 48 hours, 24 hours tol week, 3 days to 2 weeks or any intermediate, shorter or longer time period.

According to some embodiments, the NF-training comprises at least one maintenance session. Alternatively, a subject that completed the NF-training session, performs at least one maintenance session. In some embodiments, a maintenance training session comprises a stress exposure stage and a stress regulating stage, for example as in a training session of the NF training. Alternatively, the maintenance session comprises only a stress regulating stage. In some embodiments, in the maintenance session, the subject performs the at least one activity that was used during the training session, optionally, the at least one activity performed in the maintenance session is an activity that generated the maximal desired modulation on the stress response and/or on the activity level of the stress-related brain area. In some embodiments, the maintenance session is performed, for example, to keep a measurable stress response and/or at least one stress-related physiological parameter within a desired range of values or higher than a value indicative of an acute stress or chronic stress.

According to some embodiments, the maintenance session is performed at least 1 day following the completion of the NF-training, for example 1 day, 1 week, 1 month or any intermediate, shorter or longer time duration following the completion of the NF-training. In some embodiments, at least one maintenance session is performed, for example two or more maintenance sessions, 4, 6, 10, 20 or any intermediate, smaller or larger number of maintenance sessions are performed. In some embodiments, the NF training or at least some of the training sessions are performed in a clinic or in a hospital. Alternatively, the training sessions are performed outside the clinic or the hospital, for example in the house or at the workplace of the subject. In some embodiments, the maintenance session is performed in the house or at the workplace of the subject.

A potential advantage of the NF training in healthy human subjects, for example the EEG-NF, may be the ability to modify, for example to interfere, delay or block, a transition between an acute stress response and a long-standing chronic psychopathology in the subjects, or to develop a chronic psychopathology, for example PTSD in the future. An additional potential advantage of the NF training in healthy human subjects may be in increasing the ability of a subject to cope with chronic stress.

### References for the Abovementioned “Description of Specific Embodiments of the Invention”

1. Kalisch . . . Kleim, The resilience framework as a strategy to combat stress related disorders (perspective). Nature Human Behavior 1, 784-790 (2017)

2. Admon . . . Hendler, Imbalance neural responsivity to risk and reward indicates stress vulnerability in humans. Cereb Cortex 23, 28-35 (2012)

3. Admon . . . Hendler, Human vulnerability to stress depends on amygdala's predisposition and hippocampus plasticity. Proc Nat Acad Sci 106, 14120-14125 (2009)

4. Admon, Milad and Hendler, A casual model of PTSD: disentagling predisposed from acquired neural abnormalities (review). Trends Cogn Sci, 1-11 (2013)

5. Keynan, Hendler, Limbic activity modulation guided by fMRI inspired EEG improves implicit emotion regulation Biol Psychiatry 80, 490496 (2016)

6. Wang . . . Li, Synaptic modifications in the mPFC in susceptibility and resilience to stress. J Neurosci 34b, 7485-7492 (2014)

7. Maier Behavioral control blunts reactions to contemporaneous and future adverse events: mPFC plasticity and corticostriatal network. Neurobiol Stress 1, 12-22 (2015)

8. Etkin, Egner and Kalisch, Emotional processing in ACC and mPFC. Trends Cogn Sci 15, 85-93 (2011)

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Resilience Training

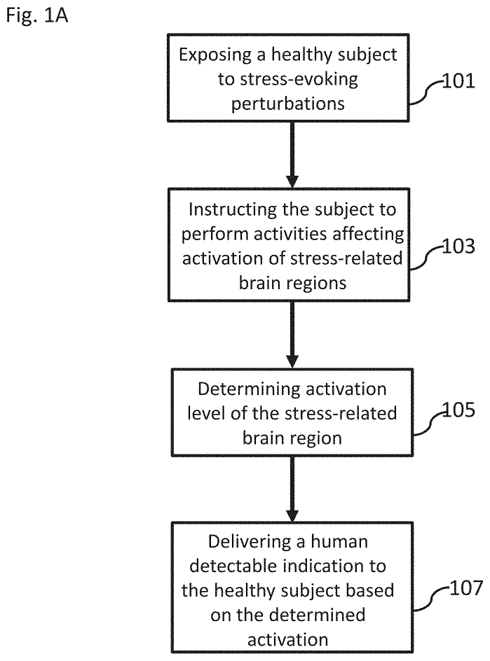

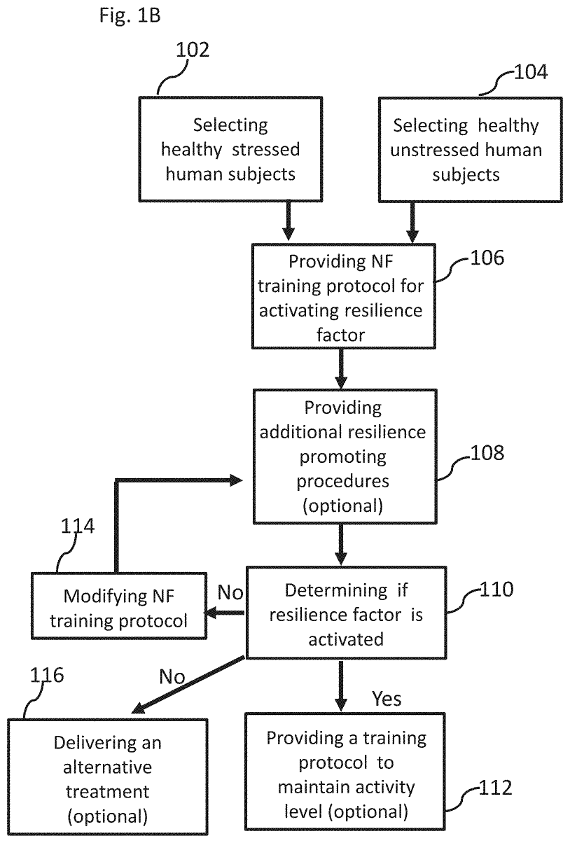

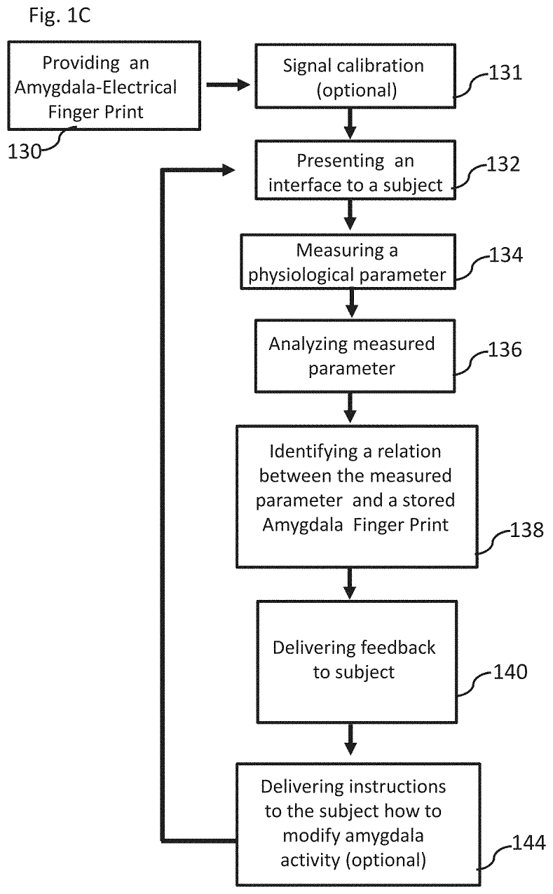

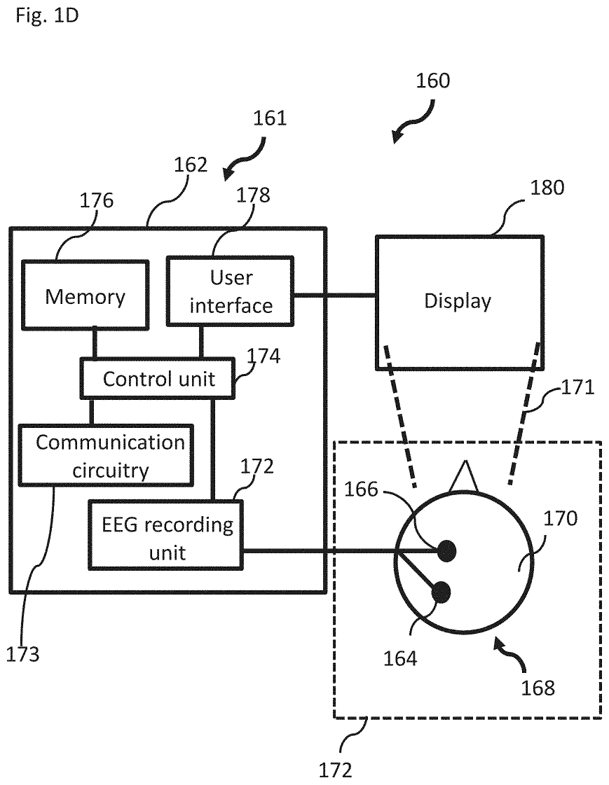

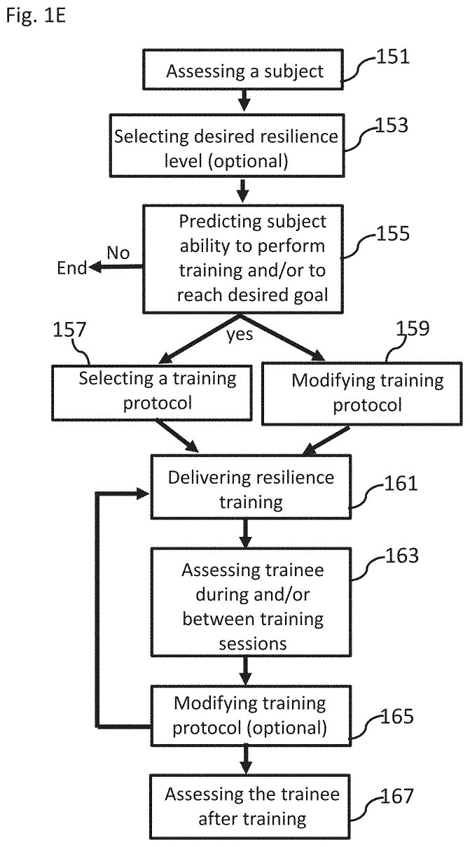

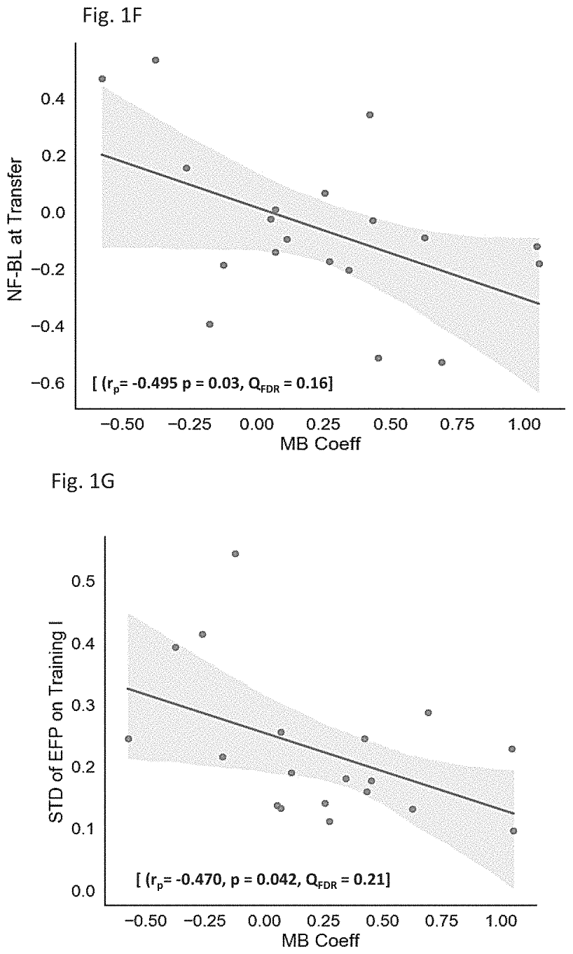

According to some exemplary embodiments, a healthy subject undergoes a resilience training procedure, for example when the subject is expected to be exposed to a stress and/or to stress-evoking perturbations. In some embodiments, a subject that is designated to practice a stressing occupation, for example subjects that are designated to become soldiers, early responders for example fire fighters, undergo the resilience training procedure. Reference is now made to FIG. 1A, depicting a general procedure for resilience training, according to some exemplary embodiments of the invention.

According to some exemplary embodiments, a healthy subject is exposed to one or more stress-evoking perturbations at block 101. In some embodiments, the perturbations are perturbations selected to induce a stress response in the subject. In some embodiments, the perturbations are selected to affect activation of at least one deeply located brain region, for example at least one brain region located within the brain under the brain cortex. In some embodiments, the at least one deeply located brain region comprises at least one limbic area, for example the amygdala. In some embodiments, the perturbations comprise human detectable perturbations. In some embodiments, the perturbations are delivered to the subject by one or more of a visual representation, sound, smell and/or by any other means that are detectable by the human subject.

According to some exemplary embodiments, the subject is instructed to perform one or more activities to affect the activation of the at least one stress-related brain region at block 103. In some embodiments, the subject performs one or more activities, for example mental or physical activities, that affect the activation of the at least one stress-related brain region. In some embodiments, the subject performs activities that downregulate an activation level of the at least one stress-related brain region, for example, activities that downregulate an activation level of the amygdala.

According to some exemplary embodiments, an activation level of the at least one stress-related brain region is determined at block 105. In some embodiments, at least one signal indicative of the activity level of the at least one brain region is recorded. In some embodiments, the at least one signal comprises an EEG signal. Alternatively or additionally, the at least one signal comprises signals recorded using fMRI. Alternatively or additionally, the at least one signal comprises an electrophysiological parameter signal. In some embodiments, the determining of the activation

According to some exemplary embodiments, the recording of the at least one signal and/or the determining of the activity level is performed in a timed relation with the exposing, for example prior to the exposing, during the exposing and/or following the exposing. In some embodiments, the activity of the at least one brain region is determined by comparing the at least one recorded signal to a stored signal or indication thereof. Alternatively or additionally, the activity of the at least one brain region is determined by comparing an activity level indicated by the at least one recorded signal to a stored activity level or indication thereof. In some embodiments, the activity level of the at least one brain region is determined using a lookup table or at least one algorithm, optionally receiving the at least one recorded signal or indication thereof, as an input.

According to some exemplary embodiments, the recorded signal is an EEG signal, recorded by at least one EEG electrode. In some embodiments, the activity level of the at least one brain region is determined based on a correlation between the recorded signal, for example an EEG signal, and a stored activity fingerprint of the at least one brain region or indication thereof. In some embodiments, the activity fingerprint is generated, for example, by calculating a correlation between one or measured EEG signals, with an activity level of the at least one brain region as monitored, for example, by fMRI.

According to some exemplary embodiments, at least one human detectable indication is delivered to the subject based on the determined activity of the at least one brain region, at block 107. In some embodiments, the at least one human detectable indication is delivered to the subject while the subject perform activities affecting the activation of the at least one brain region at 103, or following the performing of such activities. In some embodiments, the human detectable indication is delivered as a feedback to represent an ability of the subject to affect the activity level of the at least one brain region. In some embodiments, a human detectable indication indicating upregulation of the activity of the at least one brain region is different from a human detectable indication indicating down regulation of the at least one brain region. In some embodiments, different human detectable indications are used to indicate different activity levels of the at least one brain region. In some embodiments, the at least one human detectable indication comprises a visual indication, an audible indication and/or a sensory indication.

### Exemplary Resilience Factor Activation Process

According to some exemplary embodiments, resilient neural factor activation relates to changes in brain regions related to stress, for example changes in deep brain limbic areas, which increase resilience of a human subject. Alternatively or additionally, resilient neural factor activation relates to changes in neural networks which affect stress that increase resilience of a human subject. In some embodiments, the deep brain limbic areas comprise the amygdala. Reference is now made to FIG. 1B, depicting a flow chart of a process for resilient neural factor activation, according to some exemplary embodiments of the invention.

According to some exemplary embodiments, healthy stressed human subjects are selected at 102, for example healthy human subjects that encounter a stressor but are not diagnosed to have a stress-related disease or that are not treated for a stress-related disease. Alternatively, less resilient human subjects are selected at 102. In some embodiments, stressed human subjects are subjects which encountered numerous persistent stress-inducing events, for example soldiers undergoing basic training, flight cadets, early responders, or firefighters. In some embodiments, the subjects are selected based on values of at least one stress-related physiological parameter for example cortisol, heart rate variability increase, pupil dilation, an increase in SCR, increase in muscle tension of some muscles, for example some face muscles or any combination of these stress-related physiological parameters. Alternatively or additionally, the healthy stressed human subjects are selected based on self-report and/or based on an observation of an expert, for example a psychologist or a psychiatrist.

According to some exemplary embodiments, the healthy stressed human subjects are selected based on measurements of at least one physiological parameter, for example Cortisol levels, blood pressure, heart rate or any other physiological parameter related to stress. Alternatively or additionally, the healthy stressed human subjects are selected based on an expert evaluation, for example a psychologist or a psychiatrist evaluation. In some embodiments, the healthy stressed human subjects are selected based on a stress questionnaire.

Alternatively and according to some exemplary embodiments, healthy unstressed human subjects are selected at 104. Alternatively, resilient human subjects are selected at block 104. In some embodiments, the unstressed human subjects are selected prior to a predicted stress events or a series of stress events, for example events that are expected to affect the unstressed human subjects. In some embodiments, the NF training protocol is applied up to 2 months, for example up to 1 month, up to 2 weeks, up to 1 week, up to 3 days or any shorter or longer time period prior to the expected stress events or prior to the expected series of stress events.

According to some exemplary embodiments, less-resilient human subjects and more resilient human subjects are selected at block 102 and 104 respectively based on one or more stress tests, for example the Trier Social Stress Test (TSST), the Montreal Imaging Stress Task (MIST) and/or threat of obtaining painful stimuli, horror movie or virtual reality stressful scenario. In some embodiments, the NF training or at least one parameter thereof is modified according to the results of the one or more stress tests. Optionally, the one or more stress-test are performed during the NF training, for example between training sessions, for example to monitor a progress of the trainee. In some embodiments, a results of the one or more stress tests is used as a desired goal of the NF training.

According to some exemplary embodiments, a NF training protocol is provided according to the subject type at 106. In some embodiments, the NF training protocol is configured to allow activation of a resilience factor in the trainees. In some embodiments, the NF training protocol is personalized to each trainee or to a group of trainees. In some embodiments, the training protocol is personalized according to a training history of a trainee, for example outcomes of previous training sessions. In some embodiments, the training protocol is personalized according to a training protocol and/or advancement of each trainee or a group of trainees.

According to some exemplary embodiments, a NF training protocol comprises an interface, for example a scenario, a continuously changing scenario, a continuously changing environment. In some embodiments, the interface comprises a virtual reality or an augmented reality interface. In some embodiments, the interface is presented to the trainee on a display, for example a display of acellular device or any other mobile device. Optionally, the interface is presented on a wearable device or a head-mounted device, for example a head-mounted helmet or glasses.

According to some exemplary embodiments, the interface of the NF training protocol follow an activation level of at least one stress-related brain area or activity modulation of the stress-related brain area. In some embodiments, the at least one stress-related brain area comprises at least one deep limbic brain areas, for example the amygdala. In some embodiments, at least one parameter of the interfaces changes according to the activation level or the activity modulation of the stress-related brain areas. In some embodiments, the at least one interface parameter comprises, shape, color and size of the interface. Alternatively or additionally, the at least one interface parameter comprises content of the interface, objects number, objects size, objects color, objects shape and/or interaction between the objects in the interface.

According to some exemplary embodiments, in a NF training protocol for unstressed human subjects, the interface comprises one or more stressors configured to induce stress in the unstressed healthy subjects. In some embodiments, the healthy unstressed subjects negotiate the one or more stressors, for example on a display positioned in a field of view of the unstressed subjects. In some embodiments, an avatar of the trainee negotiates the one or more stressors on the display, for example in a virtual reality or in an augmented reality environment. In some embodiments, the one or more stressors comprise one or more objects presented on a display. Alternatively, the one or more stressors comprise a situation, for example a social situation presented in the interface.

According to some exemplary embodiments, during the NF training protocol, trainees perform physical and/or mental exercises configured to modulate activity of stress related brain areas, for example deep limbic brain areas. In some embodiments, the physical and/or mental exercises are performed in a timed relationship with events in the interface, for example with changes in the scenario. In some embodiments, the trainees perform the physical and/or mental exercises while or after negotiating one or more objects in the interface.

According to some exemplary embodiments, the trainees receive a feedback, for example a human detectable indication, regarding the activation level of the resilience factor and/or the activity modulation of the resilience factor following the performance of the exercises. Alternatively or additionally, the trainees receive a feedback, for example a human detectable indication, regarding the activation level of the amygdala and/or regarding the activity modulation of the amygdala following the performance of the exercises.

According to some exemplary embodiments, the feedback comprises modifying one or more parameters of the interface, for example size, shape, content, number of objects in the scenario, color of the objects, size of the objects, posture of the objects and/or interaction between the objects in the interface. In some embodiments, the feedback is delivered to the trainee only when reaching a predetermined activity level or when reaching a predetermined activity modulation of the amygdala. In some embodiments, the feedback comprises a covert feedback, for example a reward in a continuous interface which is optionally a gamified interface.

According to some exemplary embodiments, the NF training protocol is combined with additional resilience promoting procedures at 107. In some embodiments, the resilience promoting procedures comprise reappraisal training, improving response to emotional distractors optionally via emotional stroop or attention threat bias modification or EMDR.

According to some exemplary embodiments, activation level or activity modulation level of the resilience factor is determined at 110. Alternatively or additionally, activation level and/or activity modulation level of the amygdala is determined at 110. In some embodiments, the activity level and/or activity modulation level is determined by one or more tests. Alternatively or additionally, the activity level and/or activity modulation level is determined based on measurements of at least one physiological parameter, for example Cortisone, heart rate, blood pressure or any other physiological parameter.

According to some exemplary embodiments, the activity level or activity modulation level of the resilience factor is determined based on EEG signals recorded during the NF training, indicating a specific activity level or activity modulation level of at least one selected stress-related brain area, for example at least one selected brain area of the limbic system.

According to some exemplary embodiments, the ability of the subject to perform the NF training or how good the subjects perform the NF training, for example NF training efficacy, is determined at block 110. In some embodiments, the subjects receive a feedback, for example a human detectable indication regarding their ability to perform the NF training or the NF training efficacy.

According to some exemplary embodiments, if the activation level of the resilience factor or the amygdala reached a desired level or in a desired range of values, then an additional training protocol is delivered to the subject after a selected time period at 112. In some embodiments, the additional training protocol is configured to maintain the effect of the NF training protocol. Alternatively or additionally, the additional training protocol is configured to enhance the effect of the NF training protocol. Optionally, the additional training protocol does not include feedback and/or measuring the activity of brain regions. In some embodiments, the additional training protocol comprises measuring at least one physiological parameter associated with stress or with amygdala activity, for example heart rate, blood pressure, skin electrical conductivity, muscle tension, pupil dilation, facial muscle tension, densely sampled self-report, an epigenetic marker, a microbiome marker, or any other physiological parameter. According to some exemplary embodiments, if the activation level of the resilience factor or the amygdala did not reach a desired level or is not in a desired range of values, then at least one parameter of the NF training protocol is modified at 114. In some embodiments, the at least one parameter of the NF protocol comprises training protocol duration, or at least one parameter related to the interface, for example interface content, shape, number, size, color of objects in the interface, interface type, complexity of the presented environment, complexity of the interactions between two or more objects in the presented environment.

According to some exemplary embodiments, if the activation level of the resilience factor or the amygdala did not reach a desired level or is not in a desired range of values, then an alternative treatment is delivered to the subject at 116. In some embodiments, the alternative treatment comprises a drug or any other alternative treatment.

### Exemplary Amygdala-Electrical Finger Print Neurofeedback Process

According to some exemplary embodiments, an amygdala neurofeedback process, for example an amygdala-Electrical Finger Print neurofeedback (Amygd-EFP-NF) process, is delivered to a subject, optionally as a training protocol having one or more training sessions. In some embodiments, the Amygd-EFP-NF process monitors activity level or changes in activity level of stress-related brain regions. In some embodiments, in the Amygd-EFP-NF process, EEG signals are recorded and a relation to a known amygdala-Electrical Finger Print is determined. In some embodiments, based on the determined relation, an indication, for example a human detectable indication is delivered to the trained subject, also referred herein as a trainee.

According to some exemplary embodiments, the training protocol is composed of one or more training sessions, for example 2, 4, 6, 8 or any intermediate, smaller or number of training sessions. In some embodiments, the training protocol comprises 6 NF meetings. In some embodiments, at the first session the trainee is explained that the purpose of the training is to enhance stress resilience by acquiring volitional control of amygdala activity. In some embodiments, the NF trainee is instructed to find a mental state that corresponds to an ease in the unrest level of a presented scenario. Optionally, instructions are intentionally unspecific, for example to allow individuals to adopt a mental strategy that they subjectively find most efficient.

According to some exemplary embodiments, one or more training sessions or each training session includes 3 consecutive conditions, Watch, Regulate and Wash-out. Alternatively, one or more training sessions or each training session comprises only one or two conditions, for example a Regulate condition. In some embodiments, during a Watch condition, the trainee is instructed to passively view a scenario which is fixed on a predetermined agitation level, for example 50%, 60%, 70%, 75% or any intermediate, smaller or larger agitation level percentage value. In some embodiments, the agitation level is related to a specific function/model of learning or personally determined with the success (adaptive). In a Watch, for example a Rest condition, the activity level of the amygdala does not affect the presented scenario. According to some exemplary embodiments, during Regulate the trainee is instructed to find a mental strategy that corresponds an appeasement in the scenario unrest level. In some embodiments, during washout the trainee taps his thumb to his finger according to a 3-digit number that appears on the screen.

According to some exemplary embodiments, agitation level corresponds to a stress effect level generated by one or more perturbations and/or stressors. For example, in a scenario which includes sitting and standing: agitation corresponds to the ratio between characters sitting down to those protesting in the counter. 0% -all are sitting down, 100% all are standing up.

In some embodiments, during watch (or any other baseline) agitation is fixed. In some embodiments, during regulate, optionally for each online recorded EFP value, a software automatically calculates a probability of receiving this value during the watch condition (the standard score of the momentary value in the regulate condition with respect to the mean and standard deviation of the previous watch condition).

According to some exemplary embodiments, a washout condition, is a recovery condition configured to allow recovery of the brain activity, activity of one or more brain regions or neural circuits, to a baseline level.

According to some exemplary embodiments, each training session comprises one or more training cycles, for example 5 training cycles. In some embodiments, each training cycle or at least some of the training cycles include one or more of a Watch condition, a Regulate condition, and a regulate condition. In some embodiments, each condition or at least some conditions last for a time period of up to 180 seconds, for example a time period of 15 seconds, a time period of 30 seconds, a time period of 60 seconds or any intermediate, shorter or longer time period. In some embodiments, a time duration of a Watch and/or Regulate conditions is up to 120 seconds, for example 60 seconds. In some embodiments, a time duration for a washout condition is up to 60 seconds, for example 30 seconds.

According to some exemplary embodiments, at least some of the training sessions comprise training sessions without feedback. In some embodiments, in training sessions without feedback, a presented scenario is not modulated in response to amygdala activity. In some embodiments, in training sessions without feedback an agitation level of the scenario is fixed, for example on a 75% agitation level.

According to some exemplary embodiments, at least some of the training sessions comprise a cognitive training. In some embodiments, the cognitive training is performed in a timed relation with downregulation of a brain signal.

Reference is now made to FIG. 1C, depicting a detailed amygdala NF process, for example an Amygd-EFP-NF process, according to some exemplary embodiments of the invention.

According to some exemplary embodiments, a signature, for example a finger print of a limbic system indicating activity of a selected brain area, for example an Amygdala-Electrical Finger Print (Amyg-EFP) is provided at 130. In some embodiments, the Amyg-EFP, also termed herein as an Amygdala-EFP model, comprises a generic finger print, for example a fingerprint which represents activation level or activity modification levels of the amygdala in a group of subjects. In some embodiments, the Amygdala-EFP comprises EEG data. Alternatively, the fingerprint indicates an activity of at least one selected limbic system brain area, for example an activity level of the amygdala in a specific subject. In some embodiments, the finger print is extracted, for example identified from a recorded EEG data as described in US20140148657A1.

According to some exemplary embodiments, EEG data used for the Amygdala-EFP model is a Time/Frequency matrix recorded from one or more electrodes Pz, for example one or more EEG electrodes. In some embodiments, the EEG data used for the model includes all frequency bands in a sliding time window of 5-20 seconds, for example in a time window of 5 seconds, 8 seconds, 10 seconds or any intermediate, smaller or larger time window size. In some embodiments, the EEG data used for the model includes all frequency bands, for example in a range of 1-60 Hz, for example in a range of 1-30 Hz, 15-50 Hz, 40-60 Hz or any intermediate, smaller or large range of frequencies. In some embodiments, the EEG data used for the model includes all frequency bands, for example in a range of 1-60 Hz, in a sliding time window of 12 seconds. In some embodiments, to obtain the amygdala BOLD predictor, the EEG data is multiplied by the EFP model coefficients matrix. In some embodiments, the EFP model comprises of a frequency by delay by weight matrix in which every frequency band is differently weighted in different time delays.

According to some exemplary embodiments, a control unit, for example a control unit 174 shown in FIG. 1D, which optionally includes a controller, is configured to analyze EEG signals recorded during the NF training to extract the fingerprint, optionally using the steps described above and for example in US20140148657A1. In some embodiments, the control unit 174 is configured to determine an activity level of at least one brain region based on the extracted finger print, for example by identifying a correlation between the extracted finger print and a finger print or indication thereof stored in the memory 176.

According to some exemplary embodiments, one sampling unit, calculated every three seconds, contains weighted data from the last 12 seconds. A potential advantage of using the Amygdala-EFP model is that while conventional EEG measures used for NF commonly calculate the amplitude of specific band-widths or the ratio between them, the Amyg-EFP takes into account a wide spectrum of 1-60 Hz in a time window of 12 seconds.

According to some exemplary embodiments, the signature, for example a generic signature or a personalized signature, for example a signature indicating an activity of at least one selected brain region in a specific subject, is stored in a memory, for example memory 176 of NF device 16 shown in FIG. 1D.

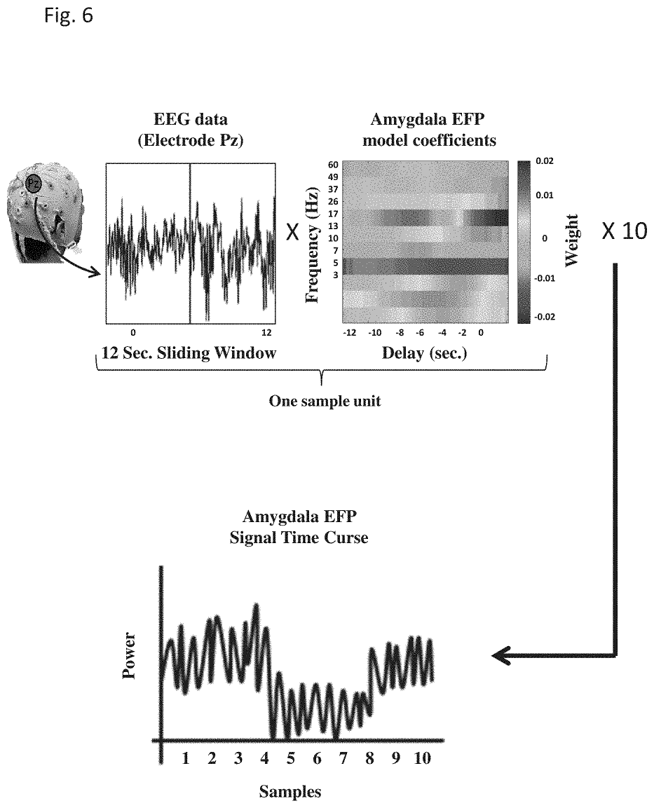

According to some exemplary embodiments, a signal calibration, for example an EEG signal calibration is performed at 131. In some embodiments, an EEG signal calibration is performed, for example to calibrate the Amyg-EFP, for example a generic Amyg-EFP with EEG signals recorded from a specific subject. In some embodiments, since the generic Amygdala-EFP model takes into account a time window of 12 seconds, each or at least some NF training sessions begin with a calibration session in which a subject views a fixed object. Alternatively, the calibration is performed on a first session of the training protocol. In some embodiments, during calibration a baseline of a subject is normalized to fit the EFP model, for example the signature. In some embodiments, calibration is performed at least 5 seconds prior to training, for example at least 5 seconds, at least 10 second, at least 12 second or any intermediate, smaller or larger value prior to the training. In some embodiments, the calibration time is determined according to duration of a sliding window of the EEG recording, for example as shown in FIG. 6.

According to some exemplary embodiments, an interface is presented to the subject at 132 and for example as described in 106. In some embodiments, the interface is presented on a display, for example using virtual reality or augmented reality techniques. In some embodiments, the interface follows an activation level of the amygdala. Alternatively, the interface follows an activation modulation level of the amygdala. In some embodiments, the interface comprises a personalized interface to a selected subject or to a group of subjects. Optionally, the interface is personalized to according to the subject occupation or every-day life environment of the subject. Optionally, the interface comprises one or more stressors, configured to induce a stress response in the subject.

According to some exemplary embodiments, at least one physiological parameter of a subject is measured at 134. In some embodiments, the at least one physiological parameter comprises a physiological parameter related to the activity level of stress-related brain regions, for example the amygdala. In some embodiments, the at least one physiological parameter comprises fMRI signals, heart rate, blood pressure and/or EEG signals.

According to some exemplary embodiments, the at least one measured physiological parameter, for example EEG signals, is analyzed at 136.

According to some exemplary embodiments, a relation between values of the analyzed physiological parameter and a fingerprint of the amygdala activity level or a modulated activity level is identified at 138. In some embodiments, a relation between the analyzed EEG signals and the amygdala finger print, for example the Amyg-EFP is determined at 138.

According to some exemplary embodiments, a feedback is delivered to the subject at 140. In some embodiments, the feedback is delivered by modifying the interface presented to the subject at 132. Alternatively or additionally, the feedback is delivered by at least one human detectable indication. In some embodiments, the feedback follows the activity level of the amygdala. Alternatively or additionally, the feedback follows the modulation level of the amygdala activity.

In some embodiments, the feedback is generated according to the identified relation at 138.

According to some exemplary embodiments, instructions are delivered to the subject at 144. In some embodiments, the instructions comprise instruction how to modulate the activity of the amygdala. Alternatively or additionally, the instructions comprise instructions how to modulate the activity of one or more additional stress-related brain regions. Optionally, the instructions comprise instructions to perform one or more mental and/or physical exercises. In some embodiments, the instructions are provided following the presentation of the interface at 132.

According to some exemplary embodiments, the instructions comprise instructions to find a mental state that lowers an agitation level presented in the interface at 132.

Exemplary Neurofeedback system

Reference is now made to FIG. 1D depicting a neurofeedback system, according to some exemplary embodiments of the invention. According to some exemplary embodiments, a neurofeedback system, for example neurofeedback system 160 is configured to deliver a NF training protocol to a human subject, for example subject 168. In some embodiments, the NF training protocol is configured to activate a resilience factor in the human subject, for example as described in this application.

According to some exemplary embodiments, the system comprises a NF device 161 and one or more electrodes, for example EEG electrodes 166 and 164. In some embodiments, the one or more EEG electrodes 164 and 166 are shaped and sized to be attached to a skull 170 of the subject 168. In some embodiments, the EEG electrodes are configured to record EEG signals from the brain of the subject 168.

According to some exemplary embodiments, the NF device 161 comprises a control unit, for example control unit 174, electrically connected to a user interface 178. In some embodiments, the user interface 178 comprises a display, for example to display an interface as described at 132 in FIG. 1C or at 106 in FIG. 1B. Alternatively, the user interface 178 is electrically connected to an external display 180, for example a screen, a head-mounted display, a virtual or augmented reality helmet, or a virtual or augmented reality glasses, positioned in the field of view 171 of the subject 168.

According to some exemplary embodiments, the control unit 174 is electrically connected to a memory 176. In some embodiments, the memory stores one or more fingerprints of the amygdala, log files of the NF device 161 and/or at least one NF training protocol or parameters thereof. In some embodiments, the control unit 174 signals the user interface 178 to present an interface to the subject 168 on the external display 180. In some embodiments, the control unit 174 signals the user interface 178 to present an interface to the subject 168, based on one or more parameters of the interface stored in the memory 176. Alternatively or additionally, control unit 174 is connected optionally by a wireless connection to a remote memory storage, for example a cloud memory. In some embodiments, the cloud memory stores one or more of information related to the interface presented to a trainee, at least one training protocol or parameters thereof, values of at least one parameter related to the performance of a trainee during the training protocol, and/or values of at least one parameter related to the activity level of the amygdala and/or to modulation of the amygdala activity.

According to some exemplary embodiments, the memory stores values of at least one parameter related to the performance of a subject, for example amygdala activity level reached during one or more selected training sessions or the highest amygdala activity level reached in a selected time period or during the training protocol. In some embodiments, the control unit 174 presents to a trainee information regarding an advancement of the trainee during the training protocol, for example based on information stored in the memory 176 and/or in the remote memory storage.

According to some exemplary embodiments, the one or more EEG electrodes 164 and 166 are electrically connected to an EEG recording unit 172 of the NF device 161. In some embodiments, the EEG electrodes deliver recorded EEG signals to the EEG recording unit 172. Optionally, the EEG recording unit comprises an amplifier which is configured to amplify the recorded EEG signals.

According to some exemplary embodiments, the control unit 174 is electrically connected to the EEG recording unit 172. In some embodiments, the control unit is configured to analyze the recorded EEG signal using one or more algorithms, for example statistical algorithms stored in the memory 176. Additionally, the control unit 174 identifies a relation between the recorded or analyzed EEG signals and a fingerprint, for example an amygdala EEG fingerprint stored in the memory 176. In some embodiments, the fingerprint is indicative of a selected activation level of a brain region, for example the amygdala. Alternatively or additionally, the fingerprint is indicative of a modulation level of the brain region. Optionally, the fingerprint is an algorithm or a look-up table that allows to translate different recorded EEG signals or portions thereof to an activity level or to an activity level modulation of a brain region, for example the amygdala.

According to some exemplary embodiments, the control unit 174 delivers a feedback to the subject according to the identified relation. In some embodiments, the feedback delivery comprises modifying the interface presented on the display, for example external display 180 according to the identified relation. In some embodiments, the interface presented on the display is modified according to one or more interface parameters stored in the memory 176.

According to some exemplary embodiments, the NF training protocol is delivered by the system 160 while the subject 168 is in a stressful environment 172, comprising external stressors. In some embodiments, the control unit 174 determines a baseline for the recorded EEG signals and/or normalizes the recorded EEG signals, for example to compensate for the stressful environment effect of the recorded EEG signals.

According to some exemplary embodiments, a generic fingerprint for the amygdala, for example a generic Amyg-EFP, is s stored in memory 176. In some embodiments, the control unit 174 is configured to calibrate the stored amygdala fingerprint based on EEG signals recorded from subject 168 under a controlled condition, for example when the subject generates EEG signals in response to a selected calibration trigger, for example when visualizing an irrelevant scenario or an irrelevant object.

According to some exemplary embodiments, the NF device 161 is a mobile device, comprising casing 162 that is shaped and sized to easily carry the NF device 161. In some embodiments, the NF device 161 comprises a power source, for example a battery. Optionally, the power source is a rechargeable power source. In some embodiments, the power source is configured to deliver electrical power to the mobile NF device, for example in remote locations.

According to some exemplary embodiments, the device 161 comprises a communication circuitry 173 electrically connected to the control unit 174. In some embodiments, the communication circuitry is configured to receive and/or to deliver wireless communication, for example Bluetooth, WiFi or any other wireless signals. Alternatively or additionally, the communication circuitry is configured to deliver information via wires.

According to some exemplary embodiments, the control unit 174 signals the communication circuitry 173 to deliver information related to a success of a subject, an activity level of one or more brain regions during a training session or following a training session, a progress report of the subject, and/or log files of the device 161. In some embodiments, the control unit 174 delivers the information to a remote computer, a remote mobile device, for example a cellular device, and/or to a remote data storage, for example a remote server.

According to some exemplary embodiments, the NF device 161 is a cellphone device. Optionally, the cellphone device is connected to the EEG electrodes 164 and 166 via an adapter or by wireless communication.

According to some exemplary embodiments, the user interface 178 presents an interface, for example a two-dimensional (2D) or a three-dimensional (3D) interacting interface to the user on the display 180. Alternatively, the user interface 178 presents the interface on a display of the

NF device 161, for example if the NF device is a mobile device, for example a cellular device then the user interface 178 displays the interface on a display of the cellular device. In some embodiments, the control unit 174 generates the interface, for example a 3D interacting interface using the “Unreal Engine” software package or any other 3D graphical engine stored in the memory 176.

### Exemplary Subject Assessment

Reference is now made to FIG. 1E, depicting a process for assessment of a subject before, during and following resilience training, according to some exemplary embodiments of the invention.

According to some exemplary embodiments, a subject, for example a healthy subject, is assessed at block 151. In some embodiments, the subject is assessed at block 151 prior to resilience training. In some embodiments, the subject assessment comprises assessment of alexithymia. In some embodiments, alexithymia level of a subject is assessed using the Toronto Alexithymia Scale (TAS-20) questionnaire. In some embodiments, scores on the Toronto Alexithymia scale (TAS-20) questionnaire indicate level of alexithymia. Additionally or alternatively, the assessment of the subject comprises quantifying learning models of the subject. In some embodiments, the learning models of the subject are quantified using a two-step task, for example as described in Daw et al., 2011. Alternatively or additionally, the assessment comprises assessment of cognitive flexibility.

According to some exemplary embodiments, a desired resilience level of a subject is optionally selected, at block 153. In some embodiments, the desired resilience level is selected based on a profession, for example a profession in which a probability of a subject to be exposed to stress and/or to stress-evoking perturbations is high or is higher than a predetermined value. Alternatively or additionally, the desired resilience level of the subject is selected based on a geographical location, for example a geographical location in which a probability of a subject to be exposed to stress and/or to stress-evoking perturbations is high or is higher than a predetermined value. Alternatively or additionally, the desired resilience level is selected according to the level of stress the subject is expected to feel and/or the intensity of stress-evoking perturbations the subject is expected to encounter.

According to some exemplary embodiments, the subject ability to perform resilience training and/or to reach a desired goal of a resilience training, for example the selected desired resilience level, is predicted at block 155. In some embodiments, the subject ability is predicted based on the results of the subject assessment performed at block 151. Alternatively or additionally, the subject ability is predicted based on a correlation between results of the subject assessment performed at block 151 and the desired resilience level selected at block 153. In some embodiments, the subject ability to perform resilience training and/or to reach a desired goal of a training is based on the alexithymia level measured at block 151. Alternatively or additionally, the subject ability to perform resilience training and/or to reach a desired goal of a training is based on the learning model quantified at block 151.

According to some exemplary embodiments, if the subject is capable to perform the resilience training and/or to reach the desired resilience level, then a resilience training protocol is selected out of two or more resilience training protocols, at block 157. In some embodiments, the selected resilience training protocol is a training protocol that is already adjusted to an alexithymia level of the subject, for example as assessed at block 151. Alternatively or additionally, the selected training protocol is a training protocol that is already adjusted according to a quantified learning model of the subject.

According to some exemplary embodiments, the resilience training protocol is selected from at least one or two or more training protocols stored in a memory 176 of the device 161, for example shown in FIG. 1D. In some embodiments, the results of the assessment performed at block 151, for example alexithymia level of the subject and or a quantified learning model of the subject, are inserted to the memory 176 via user interface 178. In some embodiments, the control unit 174 selects the training protocol or suggests two or more training protocols out of training protocols stored in the memory 176 based on the stored assessment results. Alternatively or additionally, the control unit 174 selects the training protocol or suggests two or more training protocols based on a desired resilience level selected at block 153, and inserted via the user interface 178 into the memory 176.

According to some exemplary embodiments, if the subject is capable to perform the resilience training and/or to reach the desired resilience level, a training protocol is modified, at block 159. In some embodiments, the training protocol is stored in memory 176. In some embodiments, at least one parameter of the training protocol is modified, for example number of training session, type and/or complexity of stress-inducing perturbations, starting level of the protocol, duration of one or more training sessions, duration of one or more stages of a training session, for example a rest stage, a regulate stage and/or a wash stage. Alternatively or additionally, the at least one parameter comprises an increase in difficulty between training sessions is modified at block 159.

According to some exemplary embodiments, the training protocol is modified, for example by the control unit 174, based on the results of the assessment stored in the memory 176 that were performed at block 151, for example based on the alexithymia level of a subject and/or based on the quantified learning model of the subject. Alternatively or additionally, the training protocol is modified based on a desired resilience level, for example the desired resilience level selected at block 153.

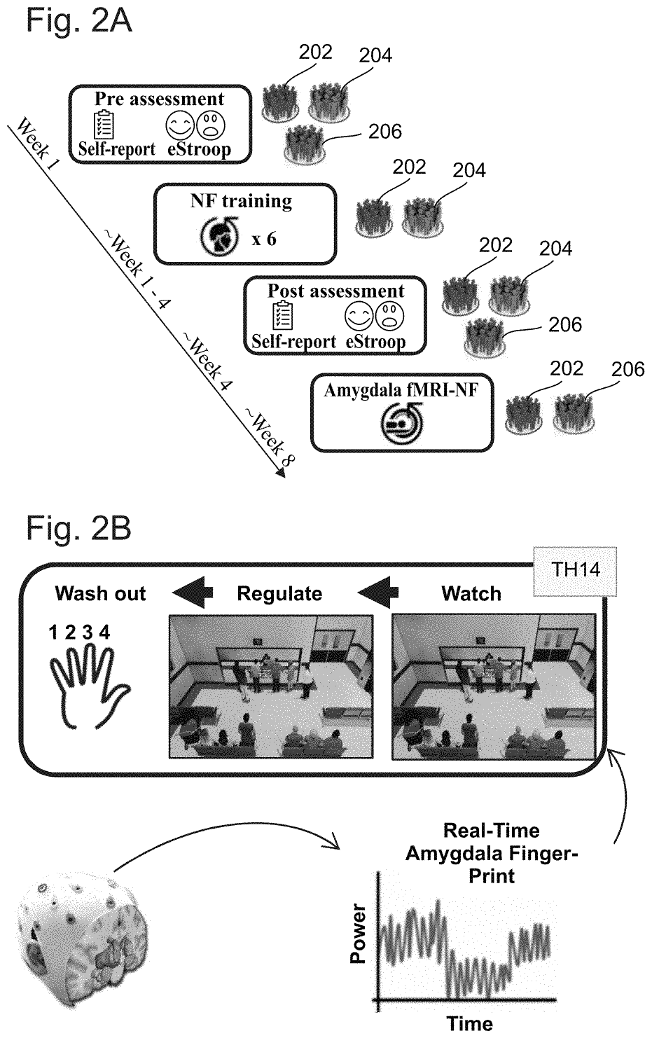

According to some exemplary embodiments, a resilience training is delivered to the subject at block 161. In some embodiments, the resilience training is delivered according to at least one training protocol or parameters thereof stored in the memory 176. In some embodiments, the control unit 174 controls the appearance and the content of each stage in a training session, for example a watch stage, a regulate stage and/or a wash out stage of a training session, for example as describe in FIGS. 2A and 2B.

According to some exemplary embodiments, the subject that performs the resilience training, for example a trainee, is assessed during and/or between training session, at block 163. In some embodiments, assessment of the subject during and/or following a training session comprises recording one or more signals, for example EEG signals, from at least one electrode, for example an EEG electrode or from an electrode array comprising two or more EEG electrodes. Alternatively or additionally, at least one physiological parameter, for example a physiological parameter indicative of activation of one or more stress-related brain regions, is recorded during and/or following a training session. In some embodiments, the assessment of the trainee at block 163 comprises determining an activation level of one or more stress-related brain regions based on the recorded one or more signals, for example EEG signals and/or the recorded electrophysiological parameter. In some embodiments, the control unit 174 is configured to record the one or more signals, to store the signals in the memory 176, and to determine the activation level of the one or more stress-related brain regions using at least one algorithm or a lookup table stored in the memory 176. According to some exemplary embodiments, the trainee assessment at block 163 comprises determining an activity level of the one or more brain regions, for example stress-related brain regions, during a rest stage, based on the recorded signals. In some embodiments, in a rest stage, the subject is instructed to passively negotiate a scenario and/or one or more perturbations, for example stress inducing perturbations, without actively performing activities designed to modify the scenario and/or the perturbations. In some embodiments, a reduction in activity level of the of the one or more brain regions between consecutive training sessions, and/or a constant decrease in activity levels, is an indication for a success of the training protocol.

According to some exemplary embodiments, an alexithymia level of the subject is assessed between and/or during a training session. In some embodiments, a change in the alexithymia level between the alexithymia level measured during and/or following a training session and a stored alexithymia level is quantified. Optionally a change in the alexithymia level compared to a baseline alexithymia level, for example the alexithymia level measured prior to training at block 151, is quantified.

According to some exemplary embodiments, the control unit 174 signals the user interface 178 to generate a human detectable indication according to the results of the assessment performed at block 163, for example according to the activity level or changes in the activity level of one or more brain regions and/or according to the alexithymia levels or changes in the alexithymia levels.

According to some exemplary embodiments, the training protocol is modified at block 165. In some embodiments, the training protocol or parameters thereof are modified based on the results of the trainee assessment performed at block 163. In some embodiments, the training protocol or parameters thereof are modified by the control unit based 174 based on the trainee assessment results stored in the memory 176.

According to some exemplary embodiments, the training protocol is modified, according to a change in alexithymia levels, for example a reduction in alexithymia level compared to an alexithymia level measured during and/or following a previous training session. In some embodiments, a constant reduction in alexithymia levels is an indication for a success of the training protocol. In some embodiments, the overall duration of the training protocol is modified based on the reduction in alexithymia levels. In some embodiments, the control unit 174 signals the user interface 178 to generate a human detectable indication, according to changes in the alexithymia levels, for example when a reduction in alexithymia levels between at least two consecutive training sessions is measured. In some embodiments, at least one parameter related to the stress-evoking perturbations, for example appearance of the perturbations, complexity and/or type of the perturbations, is modified based on the assessment performed at block 163. In some embodiments, the training protocol is modified according to a correlation between the results of the trainee assessment performed at block 163 and a desired resilience level, for example a desired resilience level selected at block 153.

According to some exemplary embodiments, the trainee is assessed at the end of the training protocol, at block 167. In some embodiments, the trainee is assessed at the end of the training protocol, for example as described at block 163. In some embodiments, the success of the resilience training is determined based on the trainee assessment performed at block 167. Alternatively or additionally, the success of the resilience training is determined based on the ability to reach a desired resilience level at the end of the training.

According to some exemplary embodiments, the control unit 174 signals the user interface 178 to generate a human detectable indication according to the results of the assessment performed at the end of the resilience training, at block 167. Alternatively or additionally, the control unit 174 signals a communication circuitry, for example communication circuitry 173 to deliver a signal, for example a wireless signal to a remote device according to the results of the assessment performed at block 167.

### Exemplary Learning Model Quantification

According to some exemplary embodiments, a learning model is quantified, for example using a two-step task. In some embodiments, the two-step task quantifies learning models by learning coefficients of model base and model free decision-making process. In some embodiments, once completed, a logistic regression is generated and used to calculate how prone each participant to learn to build a mental representation of the task, for example in order to predict outcome and his dependence on reward in action selection, for example as described in Daw et al., 2011.

According to some exemplary embodiments, the two step task is divided to two or more cycles. In some embodiments, in the first stage of each cycle a participant is asked to choose between two spaceships. In some embodiments, in the second step, the participant is asked to select between two aliens. In some embodiments, choosing a spaceship at the first step will end up in taking them to one out of two planets. In some embodiments, each spaceship would shoot to one planet most of the times, for example 70% of the time, which is referred to as the “typical” plant, while the other planet, “Atypical” would be visited by this spaceship a smaller number of times, for example, 30% of the times. The second spaceship typical and atypical planets are at the reversed order. This preference pattern is quickly learned by participants. At the second stage, once arriving to either of the planets, participants choose one of the two aliens that lives on that planet, asking them to give them a “space treasure”. Odds of a specific alien to give a prize gradually change during the progression of the game and players are directed to try and learn which of the four has the current best potential of giving the prize. The knowledge of which spaceship is likely to take them to which planet, helps participants to arrive to the planet where they would find the alien that they suspect is currently most profitable one.

According to some exemplary embodiments, for example following the methods of Daw et.al, a logistic regression analysis is used to test if participants' choice behavior (coded as change: 0; stay: 1, relative to the previous choice) was influenced by reward (coded as rewarded: 1; unrewarded: −1), transition (coded as typical: 1, atypical: −1), and their interaction, on the preceding phase. Logistic regression schematic equation:

Stay₍ₜ₎˜θ_{(MF)}*(Reward₍ₜ₋₁₎)+θ_{(MB)}*(Reward₍ₜ₋₁₎*Transition₍ₜ₋₁₎)

A main effect for reward alone indicates that there is a significant contribution of model-free learning (MF) to choice-behavior, while an interaction between Reward and Transition indicates a significant contribution of model-based (MB) learning to choice-behavior.

Reference is now made to FIGS. 1F and 1G depicting results of logistic regression analysis performed following a two-step task, as part of a validation experiment, and according to some embodiments of the invention.

FIG. 1F is a graph show results from a two-step task describing a relation between NF success and tendency for a model-based learning. FIG. 1F demonstrates a significant correlation between NF success at “transfer” and MB coefficient. Larger negative score indicates success in NF. As used herein a “transfer” is a period following the NF training in which a trainee is asked to apply the strategy that was most successful with no feedback. In some embodiments, a success in a transfer trial indicates learning and a success of the NF training.

FIG. 1G is a graph showing a relation between a standard deviation of EEG signatures, for example EEG finger prints (EFP), indicating an activity level of one or more selected brain regions. In the experiment and in some embodiments, the EEG signatures were identified in EEG signals recorded during different NF phases. FIG. 1G shows a negative correlation between model based (MB) coefficients and standard deviation (STD) of EFP values during NF training sessions. The results shown in FIG. 1G indicate that in some embodiments, an assessment of a stability level of a subject mental strategy indicates a relation between a stability level of a subject mental strategy and the ability of the subject to reach a desired goal of the resilience training and/or to succeed in the resilience training, for example the EEG-NF. Alternatively or additionally, at least one parameter, for example as described above, of the resilience training, for example the EEG-NF is modified according to the results of the stability level of the mental strategy of the subject.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Exemplary validation experiment

### Abstract

Real-time functional magnetic resonance imaging (rt-fMRI) has revived the translational perspective of NeuroFeedback (NF)¹. Particularly for stress management, targeting deeply located limbic areas involved in stress processing² has paved new paths for brain-guided interventions. However, the high-cost and immobility of fMRI constitute a challenging drawback for the scalability (accessibility and cost-effectiveness) of the approach, particularly for clinical purposes³. The current study aimed to overcome the limited applicability of rt-fMRI by using an EEG model endowed with improved spatial resolution, derived from simultaneous EEG/fMRI, to target amygdala activity (termed; Amygdala-Electrical-FingerPrint; Amyg-EFP⁴⁻⁶). Healthy individuals (n=180) undergoing a stressful military training program were randomly assigned to six Amyg-EFP-NF sessions or one of two controls (Control-EEG-NF or No-NF), taking place at the military training base. Results showed faster emotional-Stroop response and lower alexithymia scores, indicating improved emotion regulation following Amyg-EFP-NF relative to controls. Neural target engagement was demonstrated in a follow-up fMRI-NF, showing greater Amygdala-BOLD down-regulation and amygdala-vmPFC functional connectivity following Amyg-EFP-NF relative to No-NF. Together, these results demonstrate limbic specificity and efficacy on emotion-regulation of Amyg-EFP-NF during a stressful period, pointing to a scalable non-pharmacological yet neuroscience-based intervention to alleviate stress-induced psychopathology.

### Introduction

The introduction of real-time functional magnetic resonance imaging (rt-fMRI) has revived the translational interest in volitional neuromodulation via neurofeedback (NF)¹. The possibility of targeting deep-brain limbic areas such as the amygdala, known to be involved in emotional processes that are abnormal in psychopathology², has opened a new path for non-pharmacological brain guided treatment. In stress related psychopathologies in particular the down-regulation of amygdala activity via the pre-frontal- or anterior cingulate-cortex (PFC and ACC respectively) is considered a key mechanism in emotion regulation, and a feature for adaptive stress coping⁸. This pivotal role of the amygdala was recently demonstrated in a prospective study with a priori healthy soldiers⁹ by showing that amygdala hyper-activation is a predisposing factor for military stress vulnerability. Therefore, learning to regulate one's own amygdala activity may diminish detrimental- and facilitate adaptive-stress coping mechanisms.

Indeed, initial results of amygdala targeted fMRI-NF studies favorably point to the translational potential of this approach by showing strengthened amygdala-ventro-medial PFC (vmPFC) functional connectivity^{10-12,} improved emotion regulation^{4,13,14}, and reduced symptoms of major depression following treatment¹⁵. Despite the apparent promise of fMRI-NF, it's high cost, immobility and relatively low accessibility has been a challenging drawback in the scalability of this approach, especially for clinical purposes³. EEG on the other hand, is mobile and low cost but provides limited spatial specificity, particularly for deep-brain limbic areas such as the amygdala¹⁶. In a series of recent studies, the drawbacks of both imaging techniques are overcomed by applying machine learning algorithms to simultaneously recorded EEG and fMRI data^{5,6}, yielding an EEG model of weighted coefficients that could be used to probe localized Blood-Oxygen-Level-Dependent (BOLD) activity in the amygdala (hereby termed, “amygdala-Electrical Finger Print¹⁷”; Amyg-EFP; see Fug. 6). A follow-up study further validated that the Amyg-EFP can reliably probe amygdala BOLD activity, and that compared to sham-NF, Amyg-EFP-NF can lead to improved amygdala BOLD down-regulation capacities via fMRI-NF⁴. In the current study the efficacy of repeated Amyg-EFP-NF sessions on neural, cognitive and behavioral indices of emotion regulation is tested, using a double blind randomized controlled trial (RCT) with a large sample (N=180) of a-priori healthy male soldiers experiencing a stressful life period; the first weeks of combat military training^{18,19}. In order to demonstrate scalability in terms of mobility and applicability, the study took place at the soldiers' training base.

The project aimed to: (1) Demonstrate the target signal specificity of Amyg-EFP-NF relative to controls, (2) Examine the efficacy of Amyg-EFP-NF on amygdala related emotion regulation processes via anxiety²⁰ and alexithymia²¹ self-reports and performance on an emotional Stroop task²², and (3) Demonstrate target engagement of the amygdala and its cortical connections using a follow-up fMRI. To pursue the first and second aims, participants were randomly assigned to either Amyg-EFP-NF (n=90), or one of two control groups: Control-EEG-NF that probed Alpha/Theta ratio (control-NF; n=45), or No NF (NoNF; n=45). Assignment to Amyg-EFP-NF or control-NF was double blind. The Amyg-EFP-NF group underwent six NF sessions targeting Amyg-EFP down-regulation, within a period of four weeks, for example as shown in FIG. 2A. To enable a distinction between the global effects of the NF procedure and the specific effects of Amyg-EFP regulation, a control condition is designed that would account for the key common processes that underlie NF²³ (see supplementary information for more details) without targeting the neural circuit of interest (amygdala regulation and amygdala-mPFC connectivity). Therefore, similarly to the “different region” approach in fMRI-NF studies^{13,15,24}, the control-NF condition was guided by the Alpha/Theta ratio (reduced Alpha [8-12 Hz] and increased Theta [4-7 Hz]), a commonly used EEG-NF probe²⁵. Moreover, since Theta and Alpha both contribute to the Amyg-EFP model (see FIG. 6) it was imperative to further demonstrate the specificity of the Amyg-EFP on limbic processing by not only using a correlative approach, as done previously⁴, but also causally showing amygdala related behavioral changes following Amyg-EFP-NF as compared to A/T-EEG-NF alone.

Reference is now made to FIG. 6 describing the Amyg-EFP signal extraction, in the validation experiment and in some embodiments of the invention. EEG data used for the model is a Time/Frequency matrix recorded from electrode Pz including all frequency bands in a sliding time window of 12 seconds. To obtain the amygdala BOLD predictor, the EEG data are multiplied by the EFP model coefficients matrix. The EFP model consists of a frequency by delay by weight matrix in which every frequency band is differently weighted in different time delays. One sampling unite, calculated every three seconds, contains weighted data from the last 12 seconds.

While conventional EEG measures used for NF commonly calculate the amplitude of specific band-widths or the ratio between them, the Amyg-EFP takes into account the spectrum of 1-60 Hz in a time window of 12 seconds.

Reference is now made to FIG. 2A, depicting an experimental time-line of NF training, and Pre- / Post-NF assessments took place in the military training base within a period of 4 weeks. The assessments included self-report of anxiety (STAI) and alexithymia (TAS-20) and the eStroop task. Upon completion of pre-NF assessments (week 1), participants were randomized into three groups; Amyg-EFP-NF 202 (n=90), Control-NF 204 (n=45) or NoNF 206 (n=45). Amyg-EFP-NF and Control-NF conducted 6 NF session targeting down regulation of either the Amyg-EFP or a control signal (Alpha/Theta ratio) respectively, while NoNF underwent no intervention. Approximately one month following completion of NF training in the military base, a subset of 60 participants (30 Amyg-EFP-NF, 30 NoNF) underwent amygdala targeted fMRI-NF at the Sagol Brain Institute.

The control-NF group underwent the identical training protocol as the Amyg-EFP-NF group (FIG. 2A) but learned to down-regulate A/T ratio. To further control for transient psychological changes that may take place during a stressful military period, a comparison of the effect of Amyg-EFP-NF to a condition without NF training (NoNF) was performed. During the study period participants of all three groups underwent the same mandatory military training program, which took place at the same military base.

Reference is now made to FIG. 2B depicting an EEG-NF training session, during the experiment and according to some embodiments of the invention. In the experiment and in some embodiments of the invention success in down regulating the targeted signal (Amyg-EFP or Control) is reflected by audiovisual changes in the unrest level of a virtual 3D scenario (a typical hospital waiting room), manifested as the ratio between characters sitting down and those loudly protesting at the counter26,48. The NF paradigm consists of 3 consecutive conditions each repeating 5 times: Rest, for example Watch (60 sec.), Regulate (60 sec.) and Recovery, for example Washout (30 sec.). During Watch the participant is instructed to passively view the virtual scenario while it is in a constant 75% unrest level. During Regulate the participant is instructed to find the mental strategy that will lead to an appeasement in the scenario unrest level. During Washout the participant taps his thumb to his fingers according to a 3-digit number that appears on the screen.

In the experiment and according to some embodiments, a multimodal animated NF interface was used, for example to facilitate NF learning (FIG. 2B; Supplementary Video²⁶) that has been shown to optionally induce higher engagement and a more sustainable learning effect as compared to abstract visual feedback²⁶. To test for learning sustainability, participants underwent a no-feedback trial following training sessions 4-6 with the animated scenario. To further test whether learned regulation of Amyg-EFP could be transferred to situations with additional cognitive demands, upon completion of session 5, a cognitive interference trial was introduced, for example to test volitional regulation while conducting a memory task (see Supplementary Table 1 for NF trials conducted at each session). Before and after the training period all participants conducted an emotional Stroop²² (eStroop) task, testing implicit emotion regulation previously found to involve amygdala activation²⁷. In addition, all participants completed anxiety²° and alexithymia²¹ self-report questionnaires. Alexithymia refers to difficulties in cognitively processing emotions and was found related to stress vulnerability^{28,29}.

The experiment was designed to test whether Amyg-EFP-NF would result in greater Amyg-EFP down regulation relative to control-NF, and whether this learned regulation would be sustained in the absence of on-line feedback (no-feedback trial), and under the cognitive load of an irrelevant cognitive task (cognitive-interference trial). In addition, the experiment is designed whether relative to control-NF and NoNF, Amyg-EFP-NF would lead to a larger improvement in emotion regulation, as indicated by performance on the eStroop task and a greater reduction in reported anxiety and alexithymia. To pursue the third aim of neural target engagement, one month following the completion of the in-base testing, 60 participants (30 Amyg-EFP-NF; 30 NoNF) arrived at the Tel-Aviv Medical Center and underwent amygdala targeted fMRI-NF. In addition, the experiment was designed to test whether relative to NoNF, Amyg-EFP-NF would result in greater down regulation of BOLD-amygdala via fMRI-NF, and as previously shown10,12,13, d that in addition to increased down regulation of amygdala BOLD activity, Amyg-EFP-NF would result in greater amygdala-vmPFC functional connectivity.

### Exemplary Results

### Amyg-EFP-NF Learning Specificity

In the experiment and according to some embodiments, Amyg-EFP-NF success was measured as the delta of Amyg-EFP power during the active regulate condition relative to the passive watch condition (regulate—watch). The mean delta of each group in each session was subject to a 2×6 repeated measures ANOVA with NF success as the dependent variable and group (Amyg-EFP-NF vs control-NF) and session (1-6) as independent variables (See statistical analysis in the methods section for further details).

Reference is now made to FIGS. 3A-3E, describing NF learning.3A shows group difference in Amyg-EFP signal modulation across the six NF sessions. Amyg-EFP NF (red, n=88) led to a larger reduction in Amyg-EFP signal power (regulate—watch; y-axis) relative to control-NF (blue, n=38) as indicated by a significant group effect (mean difference=−0.08, se=0.02, F(1,104)=16.73, p<0.001, q2=0.13, 90% CI [0.05, 0.24]). Furthermore, as indicated by a significant group by session interaction (F(5,224)=2.39, p=0.038, η2=0.05, 90% CI [0.00, 0.08]), down-regulation of Amyg-EFP increased as the Amyg-EFP-NF training progressed, while control-NF had no such effect on the Amyg-EFP signal power. ap=0.014, bp=0.020, cp<0.001. See Supplementary Table 2B for means, sds, between group t statistics, p values, effect size estimates and CIs for each session. 3B shows a post-hoc analysis demonstrating that the Amyg-EFP-NF group reached a significant improvement in Amyg-EFP down regulation relative to the first session, from session 4 onward. FIG. 3C shows a Post-hoc analysis demonstrating that Control-NF did not result in significant changes in Amyg-EFP down regulation throughout. See Supplementary Table 3 for means, sds, within group t statistics, p values, effect size estimates and CIs comparing each session (2-6) to the first session in each group separately. FIGS. 3D-3E show NF learning sustainability. Averaged down regulation of Amyg-EFP (y-axis) during cycles with (D) the absence of online feedback in the No-Feedback condition, and when (E) conducting a simultaneous memory task in the Cognitive-Interference condition. Relative to the control-NF (blue, n=38), Amyg-EFP-NF (red, n=88) resulted in larger down regulation of amyg-EFP signal (y-axis) in both the No-Feedback condition (mean difference=−1.06, se=0.14, t(124)=7.42, p(one tailed)<0.001, d=1.44, 95% CI [1.02, 1.86]) and the Cognitive-Interference condition (mean difference=−0.09, se=0.03, t(124)=3.05, p(one tailed)=0.001, d=0.59, 95% CI [0.20, 0.98]). In FIGS. 3D and 3E, error bars indicate standard error;

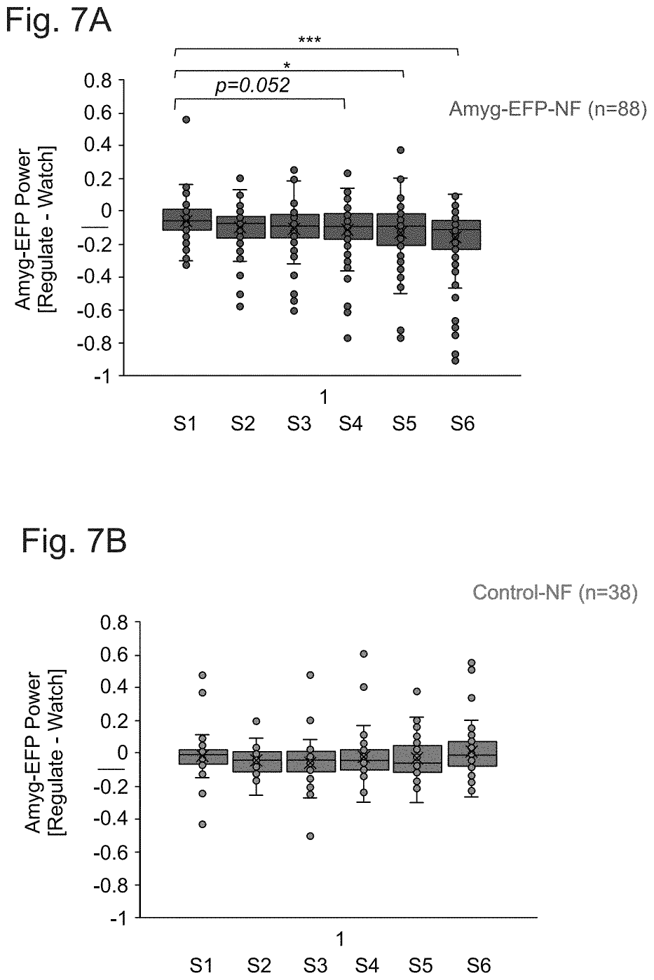

In the experiment and according to some embodiments, Amyg-EFP-NF resulted in larger Amyg-EFP down-regulation relative to control-NF (FIG. 3A), demonstrating the signal specificity of Amyg-EFP-NF training (group effect: mean group difference=−0.08, standard error (se)=0.02, F(1,104)=16.73, p<0.001, n²=0.14, 90% Confidence Interval (CI) [0.05, 0.24]). This specificity was also shown by a group-by-session interaction (F(5,224)=2.39, p=0.038, η²=0.05, 90% CI [0.00, 0.08]) means and sds of each session are reported in Supplementary Table 2A), affirming the hypothesis that Amyg-EFP-NF will lead to a larger improvement in Amyg-EFP down regulation as training progresses. The group differences reached significance at session 4 and were maintained through sessions 5 and 6 (see Supplementary Table 2B for means, SDs, between group p values, effect sizes and CIs for each session). Outlier removal (±1.5IQR; see FIGS. 7A and 7B for box plots) did not alter these results (group effect: mean difference=−0.06, se=0.01, F(1,69)=21.25, p<0.001, η²=0.24, 90% CI [0.10, 0.36] ; group by session: F(5,154)=2.33, p=0.045, η²=0.07, 90% CI [0.00, 0.12]; See Supplementary Table 5 for means and sds of each session).

In the experiment and according to some embodiments, to test which group drove the effect a post-hoc repeated measures ANOVA was conducted for each group separately, using session (S1-S6) as independent variable and Amyg-EFP-NF success (regulate—watch) as dependent variable (FIGS. 3B & 3C). A main effect of session for the Amyg-EFP-NF group (F(5,168)=3.68, p=0.003, η²=0.10, 90% CI [0.02, 0.15]) was found, with a significant linear trend (F(1,87)=18.48, p<0.001, η²=0.18, 90% CI [0.07, 0.29]). The analysis further indicated that a significant improvement relative to the first session was obtained by session 4 and was maintained throughout the last session (FIG. 3B & Supplementary Table 3). No such effect was observed for the control-NF group (FIG. 3C; F(5,122)=0.79, p=0.562, η²=0.01, 90% CI [0.00, 0.05]), nor any significant trends. See Supplementary Table 3 for means, sds t statistics, effect size estimates and CIs of within group comparison between each session (2-6) and the first session.

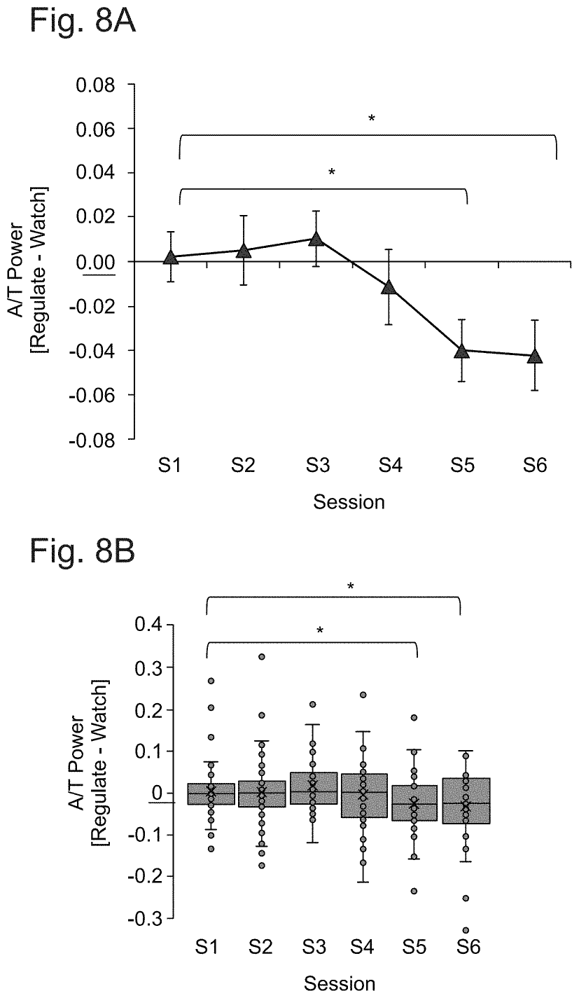

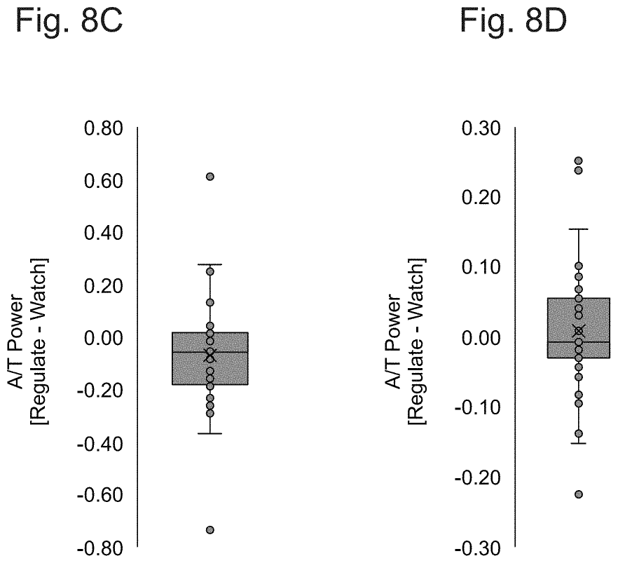

In the experiment and according to some embodiments, to verify that the control-NF group learned to down regulate the target signal (A/T), A/T signal modulations were monitored (FIGS. 8A-8D & Supplementary Table 4).

FIG. 8A describes an average change (regulate vs watch) in A/T ratio per session (S1 6), in the experiment and according to some embodiments. Significant difference from session 1 is evident at sessions 5 and 6. See Supplementary Table 4 for detailed statistics. Error bars stand for standard error. FIG. 8B describes box plots showing the distribution of A/T ratio signal modulation (y-axis; Regulate vs Watch signal power change) across the six sessions (x-axis; S1-S6). (C-D)

Box plots of control-NF learning sustainability. FIG. 8C describes a No-Feedback condition. A/T ratio down regulation was sustained in the absence of on-line feedback as indicate by a significant reduction in A/T signal (y-axis; mean (regulate—watch)=0.07±0.21, t(37)=2.19, p(one tailed)=0.014, d=0.36, 95% CI [0.02, 0.68], watch=1.41±0.41, regulate=1.34±0.43). FIG. 8D shows that while conducting a simultaneous memory task (cognitive interference condition), A/T signal reduction (y-axis; Regulate vs Watch) was not significant (mean (regulate−watch)=−0.01±0.09, t(37)=0.51, p(one tailed)=0.305, d=0.08, 95% CI [−0.24, 0.40], watch=1.05±0.19, regulate=1.06±0.22). *p<0.05 (regulate vs watch). The mean and median are marked respectively by an X and a line inside each box. Whisker lines represent 1.5X interquartile range.

FIGS. 8A-8D show, that a repeated measures ANOVA with NF success (regulate—watch) as the dependent variable and session (1-6) as independent variable revealed a significant effect of session (F(5,156)=2.92, p=0.015, η²=0.09, 90% CI [0.01, 0.14]), with significant linear (F(1,37)=6.26, p=0.017, η²=0.14, 90% CI [0.01, 0.31]) and quadratic trends (F(1,37)=4.27, p=0.046, η²=0.10, 90% CI [0.00, 0.26]). The analysis further indicated that a significant improvement relative to the first session was obtained by session 5 and maintained in session 6. See Supplementary Table 4 for means, sds, t statistics, effect size estimates and CIs of within group comparisons between each session (2-6) and the first session.

### Amyg-EFP-NF Learning Sustainability:

In the experiment and according to some embodiments, to evaluate learning sustainability participant's capacity to volitionally regulate Amyg-EFP in the absence of online feedback³⁰ (i.e. no-feedback trial; see methods) was tested. To evaluate whether the learned skill of volitional regulation is transferable to real world on-task conditions, the participants' ability to down-regulate the recorded signal while conducting a simultaneous memory task (i.e. cognitive-interference trial; see methods) was tested. Results of the no-feedback trial (FIG. 3D) demonstrated that as hypothesized, volitional regulation of Amyg-EFP could be sustained in the absence of on-line feedback, as indicated by a larger reduction of Amyg-EFP signal (regulate -watch) following Amyg-EFP-NF relative to control-NF (mean group difference=−1.06, se difference=0.14, t(124)=7.42, p(one tailed)<0.001, d=1.44, 95% CI [1.02, 1.86]; Arnyg-EFP-NF=−1.34±1.24; control-NF=−0.28±0.35). Similar results were also obtained during the cognitive-interference trial (FIG. 3E), further indicating that the Amyg-EFP signal could be regulated while conducting a simultaneous cognitive task (mean group difference=−0.09, se difference=0.03, t(124)=3.05, p(one tailed)=0.001, d=0.59, 95% CI [0.20, 0.98]; Arnyg-EFP-NF=−0.13±0.23; control-NF=−0.03±0.10). This result suggests that Amyg-EFP-NF learning is maintained even in face of additional cognitive demands.

In the experiment and according to some embodiments, to test whether volitional regulation during the no-feedback and cognitive-interference trials was successful in each group separately, the power of the targeted signal during regulate relative to watch (A/T for control-NF and Amyg-EFP for Amyg-EFP-NF) was compared. During the no-feedback trial as expected, both groups showed a significant reduction in signal power during regulate relative to watch (Amyg-EFP-NF: mean (regulate—watch)=−1.34±1.24, t(87)=10.15, p(one tailed)<0.001, d=1.08, 95% CI [0.82, 1.34], watch=−0.12±0.14, regulate=−1.46±1.23; control-NF: mean (regulate—watch)=0.07±0.21, t(37)=2.19, p(one tailed)=0.014, d=0.36, 95% CI [0.02, 0.68], watch=1.41±0.41, regulate=1.34±0.43). However, during cognitive-interference only down regulation of the Amyg-EFP was found to be feasible (Amyg-EFP-NF: mean (regulate—watch)=−0.13±0.23, t(87)=5.03, p<0.001, d=0.54, 95% CI [0.31, 0.76], watch=−1.04±1.29, regulate=−1.17±1.35; control-NF: mean (regulate—watch)=−0.01±0.09, t(37)=0.51, p(one tailed)=0.305, d=0.08, 95% CI [−0.24, 0.40], watch=1.05±0.19, regulate=1.06±0.22). Results of the memory task showed that on average participants answered 11.09±1.55 out of 13 questions correctly, with no group differences (mean difference=0.17, se=0.32, t(102)=0.54, p=0.591; d=0.11, 95% CI [−0.30, 0.52], Amyg-EFP-NF: 11.14±1.56; control-NF: 10.97±1.54), possibly suggesting a ceiling effect for cognitive load.

### Amyg-EFP-NF Training Efficacy

In the experiment and according to some embodiments, to evaluate the efficacy of Amyg-EFP-NF in modifying emotion regulation changes in performance on the eStroop task and in self-reports of anxiety and alexithymia (see methods) were measured.

Reference is now made to FIGS. 4A-4E are graphs describing outcomes of NF training per group, in the experiment and according to some embodiments. FIG. 4A describes group by time (Pre vs Post) interaction (F(2,164)=5.00, p=0.008, q2=0.06, 90% CI [0.01, 0.12]) showing that Amyg-EFP-NF (red, n=88) resulted in improved eStroop performance (y-axis; mean (post-pre)=−9.97±38.27, t(87)=2.45, p(one tailed)=0.008, d=0.26, 95% CI [0.05, 0.47]), while the control groups (control-NF [blue, n=38], NoNF [gray, n=43]) showed the opposite pattern mean (post-pre)=4.16±43.15, t(37)=0.59, p=0.553, d=0.10, 95% CI [−0.22, 0.41]; NoNF: mean (post-pre)=10.27±28.07, t(42)=2.40, p=0.017, d=0.37, 95% CI [0.06, 0.67]). FIG. 4B describes that eStroop improvement (y-axis) was grater following Amyg-EFP-NF relative to Control-NF (mean difference=−14.13, se=7.72, t(124)=1.83, p(one tailed)=0.034, d=0.36, 95% CI [−0.03, 0.74]), as well as, NoNF (mean difference=−20.24, se=6.57, t(129)=3.08, p(one tailed)=0.001, d=0.57, 95% CI [0.20, 0.94]. FIGS. 4C-4E describe Alexithymia rating changes. FIG. 4C describes group by time interaction (F(2,164)=10.69, p<0.001, q2=0.12, 90% CI [0.04, 0.19]), showing that Amyg-EFP-NF training (red, n=88) resulted in reduced alexithymia ratings (y-axis; mean (post-pre)=−3.37±9.19, t(87)=3.43 p(one tailed)<0.001, d=0.37, 95% CI [0.15, 0.58]), while the control groups showed no change (Control-NF (n=38): mean (post-pre)=0.01±7.27, t(37)=0.01 p=0.994, d=0.01, 95% CI [−0.07, 0.07]) or the opposite pattern (NoNF (n=43): mean (post-pre)=6.11±13.57, t(42)=2.96, p=0.003, d=0.45, 95% CI [0.13, 0.76]). FIG. 4D describe Alexithymia score changes with time (y-axis), showing that the reduction exhibited by the Amyg-EFP-NF group was greater compared both to control-NF (mean difference=−3.38, se=1.69, t(124)=2.00, p(one tailed)=0.023, d=0.39, 95% CI [0.00, 0.77]) and NoNF (mean difference=−9.48, se=2.29, t(129)=4.14, p<0.001, d=0.77, 95% CI [0.39, 1.15]). FIG. 4E is a scatterplot showing that the best performance out of the six Amyg-EFP-NF training (x-axis) correlated (r=0.35, p=0.002, 95% CI [0.15, 0.52]) to the reduction in alexithymia ratings (y-axis) within the Amyg-EFP-NF group only. In FIGS. 4A-4E, error bars represent standard error;

In the eStroop task, performed in the experiment and in some embodiments, participants viewed fearful or happy facial expressions with superimposed congruent or incongruent words (“happy”\“fear”) and were asked to identify the emotional expression while ignoring the words that appeared. The emotional Stroop task provides a measure of ‘emotional conflict regulation’ indicated by the difference in response times between congruent and incongruent stimuli and of ‘emotional conflict adaptation’ calculated as the difference in response times between two consecutive incongruent stimuli [ii] and incongruent stimulus following congruent stimulus [ci] (adaptation=[ii]−[ci]²². Comparing the post- vs pre-NF eStroop performance of each group revealed that as hypothesized, Amyg-EFP-NF led to a greater improvement in ‘emotional conflict regulation’ (incongruent—congruent) relative to the control groups (FIG. 4A). A group (Amyg-EFP-NF, control-NF, NoNF) by time (pre- vs post-training) interaction (F(2,164)=5.00, p=0.008, η²=0.06, 90% CI [0.01, 0.12], means and sds of each time point are reported in Supplementary Table 6) revealed that while Amyg-EFP-NF led to improved ‘emotional conflict regulation’ following training, control-NF had no effect and NoNF resulted in reduced conflict regulation post- vs pre-training (Amyg-EFP-NF: mean (post-pre)=−9.97±38.27, t(87)=2.45, p(one tailed)=0.008, d=0.26, 95% CI [0.05, 0.47]; control-NF: mean (post-pre)=4.16±43.15, t(37)=0.59, p=0.553, d=0.10, 95% CI [−0.22, 0.41]; NoNF: mean (post-pre)=10.27±28.07, t(42)=2.40, p=0.017, d=0.37, 95% CI [0.06, 0.67]). No group effect was observed (F(2,164)=1.93, p=0.148, η²=0.02, 90% CI [0.00, 0.07]) and no a-priori differences in emotional conflict regulation were observed between the Amyg-EFP-NF group and the control-NF (mean difference=5.92, se=6.12, t(124)=0.97, p=0.333, d=0.19, 95% CI [−0.19, 0.57]) or NoNF (mean difference=2.22, se=5.54, t(129)=0.40, p=0.689, d=0.07, 95% CI [−0.29, 0.44]) groups.

In the experiment and according to some embodiments, to test the main hypothesis, that Amyg-EFP-NF would lead to a larger improvement in emotional conflict regulation relative to each of the control groups separately, a post-hoc analysis was conducted comparing the change in conflict regulation (post vs pre). As hypothesized, the improvement in emotional conflict regulation (FIG. 4B) was larger following Amyg-EFP-NF compared to control-NF (mean difference=−14.13, se=7.72, t(124)=1.83, p(one tailed)=0.034, d=0.36, 95% CI [−0.03, 0.74]) and NoNF (mean difference=−20.24, se=6.57, t(129)=3.08, p(one tailed)=0.001, d=0.57, 95% CI [0.20, 0.94]; Amyg-EFP-NF=−9.97±38.27; control-NF=4.16±43.15; NoNF=10.27±28.07). No correlations were found between improvement in emotional conflict regulation and Amyg-EFP (Amyg-EFP-NF: r=0.04, p=0.742, 95% CI [−0.17, 0.25]; control-NF: r=−0.05, p=0.787, 95% CI [−0.36, 0.27]) or A/T (Amyg-EFP-NF: r=0.06, p=0.629, 95% CI [−0.15, 0.27]; control-NF: r=0.14, p=0.436, 95% CI [−0.19, 0.44]) signal reductions. Contrary to the hypothesis, no differences were found between the groups in ‘Emotional Conflict Adaptation’ ([ci]-[ii]) post- vs pre-training, as shown by an insignificant group (Amyg-EFP-NF, control-NF, NoNF) by time (pre vs post) interaction (F(2,164)=0.90, p=0.410, η²=0.01, 90% CI [0.00, 0.04], means and sds of each time point are reported in Supplementary Table 6).

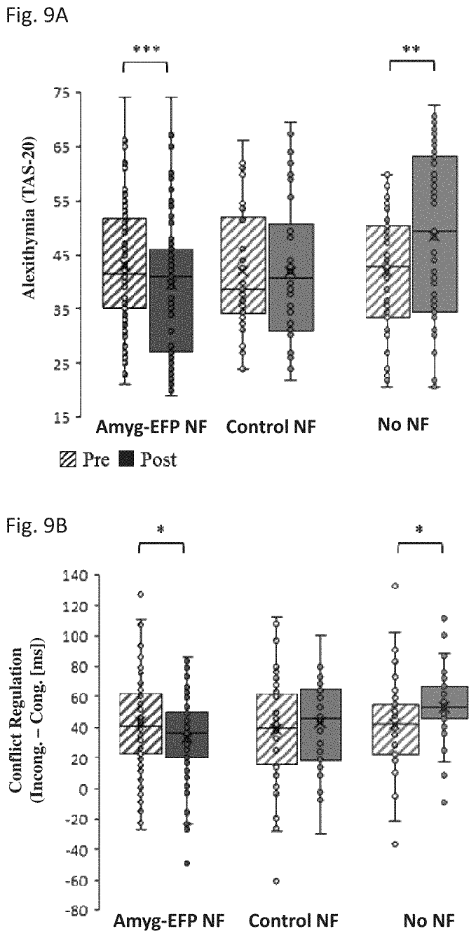

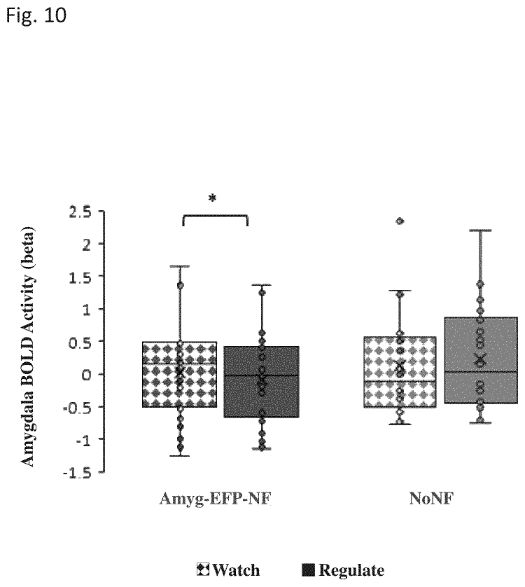

Reference is now made to FIGS. 9A and 9B showing box blots that describe the distribution of (9A) alexithymia ratings and (9B) eStroop performance before (dashed bars) and after (solid filled bars) NF training for each group [Amyg-EFP NF (red; n=88), Control-NF (blue; n=38), NoNF (grey; n=43)], according to a validation experiment and according to some embodiments of the invention; The mean and median are marked respectively by an X and a line inside each box. Whisker lines represent 1.5× interquartile range;

The distribution of TAS-20 scores at baseline was consistent with previous reports of alexithymia prevalence among healthy populations^{21,31-33} (mean=42.50±11.02). No alexithymia was exhibited by 72.8% of the sample (scores lower than 51²¹), 27.2% indicated moderate alexithymia (scores≥51) and less than 5% showed high alexithymia (scores >61; see FIG. 9A-9B). Consistent with the hypothesis, Amyg-EFP-NF resulted in a larger reduction of alexithymia scores relative to controls (FIG. 4C) as indicated by a group (Amyg-EFP-NF, control-NF, NoNF) by time (pre- vs post-training) interaction (F(2,164)=10.69, p<0.001, η²=0.12, 90% CI [0.04, 0.19], means and sds of each time point are reported in Supplementary Table 6). Interestingly, while the control-NF group showed no differences following the training period, the NoNF group showed increased alexithymia (Amyg-EFP-NF: mean (post-pre)=−3.37±9.19, t(87)=3.43 p(one tailed)<0.001, d=0.37, 95% CI [0.15, 0.58]; control-NF: mean (post-pre)=0.01±7.27, t(37)=0.01 p=0.994, d=0.01, 95% CI [−0.07, 0.07]; NoNF: mean (post-pre)=6.11±13.57, t(42)=2.96, p=0.003, d=0.45, 95% CI [0.13, 0.76]). No group effect (F(2,164)=1.64, p=0.198, η²=0.02, 90% CI [0.00, 0.06]) or a-priori differences in alexithymia were observed between the Amyg-EFP-NF group and the control-NF group (mean difference=0.96, se=2.16, t(124)=0.45, p=0.655, d=0.09, 95% CI [−0.29, 0.47]) or NoNF group (mean difference=0.95, se=2.07, t(129)=0.46, p=0.645, d=0.09, 95% CI [−0.28, 0.45]).

In the experiment and according to some embodiments, to test the main hypothesis, that Amyg-EFP-NF would lead to a larger reduction in alexithymia ratings relative to each of the control groups separately, a post-hoc analysis was conducted comparing the change in alexithymia scores (post-pre). As hypothesized, the reduction (post vs pre) was greater for the Amyg-EFP-NF group (FIG. 4D) as compared to control-NF (mean difference=−3.38, se=1.69, t(124)=2.00, p(one tailed)=0.023, d=0.39, 95% CI [0.00, 0.77]) and NoNF (mean difference=−9.48, se=2.29, t(129)=4.14, p<0.001, d=0.77, 95% CI [0.39, 1.15]; Arnyg-EFP-NF=−3.37±9.19; control-NF=0.01 ±7.27; NoNF=6.11±13.57). A Pearson correlation further demonstrated the association between the changes in alexithymia scores and Amyg-EFP-NF training (FIG. 4E), by showing that the change over time in alexithymia self-reports (post-NF-pre-NF) corresponded (r=0.35, p=0.002, 95% CI [0.15, 0.52]) with the participants best NF session (i.e. maximum Amyg-EFP reduction out of six sessions; see supplementary information). This correlation was found among participants who trained with Amyg-EFP-NF, and not among control-NF (r=0.09, p=0.644, 95% CI [−0.24, 0.40]). Furthermore, learned regulation of A/T (control-NF) did not correlate with reduced alexithymia (r=0.07, p=0.670, 95% CI [−0.11, 0.24]), nor did oscillations in the Theta (r=−0.07, p=0.441, 95% CI [−0.24, 0.11]) or Alpha (r=−0.10, p=0.288, 95% CI [−0.27, 0.08]).

A post-hoc analysis suggested that the differences between the groups in alexithymia reduction was driven by individuals who showed moderate-severe alexithymia at baseline (i.e. equal to or higher than a score of 51). This was tested by comparing between and within group differences in alexithymia reduction post- vs pre-NF, while excluding participants with a score lower than 51 pre-NF (Amyg-EFP-NF n=24; control-NF n=12; NoNF n=10). A paired samples t-test revealed a significant reduction in alexithymia scores, but only among those who underwent Amyg-EFP-NF (Amyg-EFP-NF: mean (post-pre)=−10.75±11.73, t(23)=4.49, p<0.001, d=0.92, 95% CI [0.43, 1.39], pre-NF=57.29±5.75, post-NF=46.54±13.59; control-NF: mean (post-pre)=0.25±6.47, t(11)=0.13, p=0.893, d=0.04, 95% CI [−0.53, 0.60], pre-NF=55.50±4.60, post-NF=55.75±8.17; NoNF: mean (post-pre)=0.56±4.43, t(9)=0.40, p=0.691, d=0.13, 95% CI [−0.50, 0.75], pre-NF=55.50±2.55, post-NF=54.94±5.72). This analysis further revealed that this reduction in alexithymia following Amyg-EFP-NF was larger relative to both control-NF (mean difference=−11.00, se=3.65, t(34)=3.01, p=0.003, d=1.06, 95% CI [0.32, 1.79]) and NoNF (mean difference=−10.19, se=2.78, t(32)=3.67, p<0.001, d=1.38, 95% CI [0.56, 2.18]).

Contrary to the hypothesis, an insignificant group (Amyg-EFP, control-NF, NoNF) by time (pre vs post NF) interaction (F(2,152)=0.63, p=0.530, η²=0.01, 90% CI [0.00, 0.04], means and sds of each time point are reported in Supplementary Table 6) indicated no between group differences in post- vs pre-NF self-reports of state anxiety. Interestingly however, a time effect (mean (post-pre)=−2.04±9.80, F(1,150)=6.25, p=0.013, η²=0.04, 95% CI [0.00, 0.10]; pre=32.58±9.41, post=30.54±8.11) indicated a reduction in state anxiety that was significant only for the Amyg-EFP-NF and control-NF groups but not for the NoNF group, possibly pointing to a non-specific effect of NF training (Amyg-EFP-NF: mean (post-pre)=−2.25±9.57, t(87)=2.21 p(one tailed)=0.014, d=0.24, 95% CI [0.02, 0.45]; control-NF: mean (post-pre)=−3.25±8.40, t(37)=2.38 p=0.017, d=0.39, 95% CI [0.05, 0.71]; NoNF: mean (post-pre)=−0.62±10.02, t(42)=0.40, p=0.687, d=0.06, 95% CI [−0.24, 0.36]). No group effect (F(2,162)=1.09, p=0.340, η²=0.01, 90% CI [0.00, 0.05]; means and sds of each time point are reported in Supplementary Table 6) nor a-priori differences in state anxiety were observed between the amyg-EFP group and the control-NF group (mean difference=−2.01, se=1.89, t(124)=1.06, p=0.287, d=0.21, 95% CI [−0.18, 0.59]) or the NoNF group (mean difference=−1.35, se=1.73, t(129)=0.78, p=0.434, d=0.15, 95% CI [−0.22, 0.51]). No correlations were found between reductions in state-anxiety and Amyg-EFP (Amyg-EFP-NF: r=0.16, p=0.136, 95% CI [−0.05, 0.36]; Control-NF: r=−0.06, p=0.769, 95% CI [−0.37, 0.26]) or A/T oscillations (Amyg-EFP-NF: r=0.01, p=0.966, 95% CI [−0.20, 0.22]; Control-NF: r=0.01, p=0.950, 95% CI [−0.31, 0.33]).

### Amyg-EFP-NF Related Target-Engagement

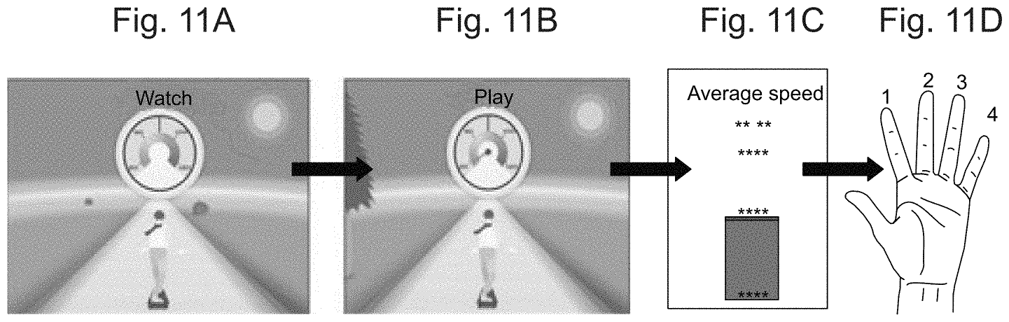

In the experiment and according to some embodiments, to test engagement of the targeted brain mechanism participants' ability to volitionally regulate Amygdala-BOLD activity via fMRI-NF was assessed. One month following the training period 60 participants (30 Amyg-EFP-NF; 30 NoNF) underwent amygdala targeted fMRI-NF with a similar design to Amyg-EFP-NF but with a different NF interface (FIGS. 11A-11D). Beta weighted activity of the targeted amygdala functional cluster during regulate relative to watch was subjected to a region of interest (ROI) analysis.

5A-5C describing results of Amygdala-fMRI-NF, one month following Amygdala-EFP-NF, in the validation experiment and according to some embodiments of the invention. FIG. 5A shows group by condition interaction (F(1,54)=10.77, p=0.002; q2=0.17, 90% CI [0.04, 0.31]) demonstrating that relative to NoNF (grey, n=26), Amyg-EFP-NF (red, n=30) resulted in greater down regulation of Amygdala BOLD activity (y-axis) during fMRI-NF (watch vs regulate). Only the Amyg-EFP-NF group, exhibited reduced amygdala BOLD activity (y-axis) during regulate (solid filled bars) relative to watch (dashed filled bars) (Amyg-EFP-NF: mean (regulate—watch)= 0.11±0.24, t(29)=2.55, p(one-tailed)=0.008; d=0.47, 95% CI [0.08, 0.84]; NoNF: mean (regulate—watch)=0.11±0.25, t(25)=2.11, p=0.045, d=0.41, 95% CI [0.01, 0.81]). FIG. 5B is a scatterplot showing that the maximum value of Amyg-EFP down-regulation across the six training sessions (x-axis) predicted (r=0.43, p=0.016, 95% CI [0.08, 0.68]) the ability to down regulate Amygdala-BOLD activity during fMRI-NF one month later (y-axis). FIG. 5C is a whole brain PPI analysis with amygdala as a seed region, showing that Amyg-EFP-NF compared to NoNF, resulted in higher amygdala-vmPFC functional connectivity during watch and regulate. In FIG. 5A, error bars represent standard error;

FIG. 5A shows that, as hypothesized, Amyg-EFP-NF resulted in better down regulation of amygdala BOLD activity, as indicated by a group (Amyg-EFP-NF vs NoNF) by condition (regulate vs watch) interaction (F(1,54)=10.77, p=0.002; n²=0.17, 90% CI [0.04, 0.31]; Amyg-EFP-NF: watch=0.03 ±0.67, regulate=−0.08±0.67; NoNF: watch=0.17±0.69, regulate=0.28±0.73). Also consistent with the hypothesis, down regulation of amygdala BOLD activity was successful only following Amyg-EFP-NF (Amyg-EFP-NF: mean (regulate—watch)=−0.11±0.24, t(29)=2.55, p(one-tailed)=0.008; d=0.47, 95% CI [0.08, 0.84]; NoNF: mean (regulate—watch)=0.11±0.25, t(25)=2.11, p=0.045, d=0.41, 95% CI [0.01, 0.81]). A Pearson correlation further revealed that participants' best performance during Amyg-EFP-NF training predicted amygdala BOLD down regulation (regulate vs watch) during fMRI-NF (r=0.43, p=0.016, 95% CI [0.08, 0.68]; FIG. 5B). This correlation was shown to be specific to Amyg-EFP and was not observed for changes in Theta (r=0.01, p=0.945, 95% CI [−0.35, 0.37]), Alpha (r=−0.01, p=0.996, 95% CI [−0.37, 0.35]) or A/T ratio (r=−0.02, p=0.911, 95% CI [−0.38, 0.34]).

In the experiment and according to some embodiments, to examine whether improved down-regulation of the amygdala during fMRI-NF could be explained by a reduction in state anxiety, as observed following both Amyg-EFP-NF and control-NF, a correlation between changes in anxiety ratings, amygdala-BOLD down regulation and learned control over A/T ratio within the group that conducted follow-up fMRI was tested. The analysis showed no correlation between A/T regulation and anxiety reduction (r=−0.02, p=0.885, 95% CI [−0.38, 0.34]) nor between anxiety reduction and follow-up amygdala BOLD down-regulation (r=0.03, p=0.883, 95% CI [−0.34, 0.39]).

In the experiment and according to some embodiments, to examine the assertion regarding the neural mechanism of amygdala down regulation capacity the targeted amygdala cluster was used as a seed region in a whole brain Psycho-Physical Interaction (PPI) analysis with group (Amyg-EFP-NF vs NoNF) and condition (regulate vs watch) as independent variables. This analysis revealed that relative to NoNF, Amyg-EFP-NF led to higher amygdala—vmPFC functional connectivity (FIG. 5C) during both the regulate and watch conditions (vmPFC peak voxel: x=9, y=62, z=−2, p(FDR)<0.05).

### Discussion

The current work conducted a multi-level investigation of a scalable (mobile, cost effective and applicable) NF method for the modulation of deeply located limbic activity, performed as an RCT among young healthy individuals during a particularly stressful life period. The Amyg-EFP computational approach for targeting limbic activity allowed us to conduct repeated NF sessions at the soldiers' base, using a large sample with multiple controls. Comparing Amyg-EFP-NF to active (control-NF) as well as NoNF controls provided careful differentiation between the specific and non-specific effects of the NF training. Relative to control-NF, Amyg-EFP-NF led to greater learning of Amyg-EFP signal reduction during training (FIGS. 3A-3C), which was maintained in the absence of online feedback and when under cognitive interference (FIGS. 3D & 3E). Reference is now made to FIG. 3F, describing a change in activity of a stress-related brain region, for example the amygdala, during a rest stage of a resilience training session, as demonstrated in the validation experiment and according to some exemplary embodiments of the invention. In the experiment, prior to each of the six Amyg-EFP NF sessions participants had a 3 minutes eyes open resting state EEG. For each participant the mean EFP amplitude across the 3 minutes was calculated. As can be seen in the FIG. 3F, the resting Amyg-EFP amplitude decreased among participants who practiced Amyg-EFP NF 322, compared to participants who practiced a control NF 324. In the control NF, the trainees are trying to lower Alpha and increase Theta, and the feedback is a ration between these continuous measurements. As shown in FIG. 3F, Amyg-EFP NF resulted in lower Amyg-EFP amplitude during rest. The rest condition was of 3 minutes long eyes open and took place at the beginning of each training session, prior to NF training. No a-priori differences were observed between the groups (Session 1 p>0.2). Within the Amyg-EFP group a significant difference relative to session 1 was observed in sessions 3 through 6 (All p<0.01). Control NF showed no differences in EFP amplitude between sessions.

According to some exemplary embodiments of the invention, monitoring a decrease in the activity of the amygdala during rest stages of two or more consecutive training sessions serves as a marker to the success of the resilience training. Alternatively or additionally, a specific activation level of the amygdala when a subject is in rest, for example during a rest stage of a training session, is used as a desired goal of the resilience training. In some embodiments, the ability of a subject to reach a desired goal of the training, for example a desired activity level of the amygdala or other stress-related brain regions during rest or when exposed to stress, is predicted based on an initial assessment of the subject prior to the training, for example as described at block 151 shown in FIG. 1E.

The efficacy of Amyg-EFP-NF training with regards to emotion regulation was tested, and a greater improvement in emotional conflict regulation (FIGS. 4A & 4B), and in self-reports of alexithymia (FIGS. 4C & 4D) following Amyg-EFP-NF, relative to controls was observed. Lastly, follow-up fMRI-NF performed on a subset of the sample, one month after completion of Amyg-EFP-NF training, demonstrated target engagement by showing that Amyg-EFP-NF resulted in a better ability to volitionally down regulate amygdala BOLD and stronger amygdala-vmPFC functional connectivity relative to NoNF (FIGS. 5A-5C). Together, the results confirm the specificity and efficacy of Amyg-EFP-NF training for emotional regulation modification under stressful life conditions.

### Amyg-EFP-NF Learning

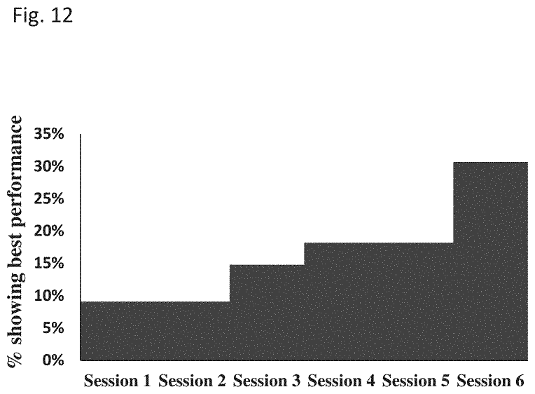

Consistent with previous studies^{15,34}, an analysis of the NF performance across the six sessions positively demonstrated that volitional brain activity regulation is a learned skill that can improve as training progresses (FIGS. 3A-3E). The control-NF did not influence the Amyg-EFP signal, demonstrating training specificity. A closer look at the results in FIG. 3A however, shows that Amyg-EFP-NF and control-NF showed a similar pattern of increased Amyg-EFP down regulation until the third session. The specificity of Amyg-EFP-NF is evident in sessions 4-6, demonstrating the importance of repeated NF sessions to achieve specificity. Also consistent with previous studies, some degree of Amyg-EFP down regulation was already observable at the end of the first session³. Nevertheless, the current results show that NF improvement did not reach plateau, what may suggest that more sessions could allow for the full realization of individual learning potential. This assumption is supported by the finding that most individuals attained their best performance during the last session (FIG. 12). If one considers that the best performance predicted both a reduction in alexithymia and follow-up amygdala BOLD down-regulation (FIGS. 4E & 5B), additional sessions could presumably result in stronger correlations and a larger influence on the outcome measures. This might be critical when moving forward to clinical populations. Thus, future studies should make use of the enhanced applicability of the Amyg-EFP approach by testing dose effect in a systematic manner, while considering a longer training period and different session intervals³⁵.

The learned ability to regulate the Amyg-EFP was sustainable in the absence of online feedback (no-feedback trial; FIG. 3D) and transferred to situations with additional cognitive demands, as demonstrated by the cognitive-interference trial (FIG. 3E). However, while the learned regulation of the targeted control signal (A/T) following control-NF was sustained during the no-feedback trial (FIG. 8C), it was not transferable to the cognitive-interference trial (FIG. 8D). Given the nature of the targeted signal in control-NF (elevation of slow wave Theta power and lowering Alpha power), it is possible that the induction of fast wave activity via a memory task hampered volitional regulation of the A/T ratio. One might therefore argue that this difference in regulation during cognitive-interference hampers the comparisons that could be made between the groups. It should be noted however that cognitive-interference was introduced upon completion of the NF training (without cognitive-interference) at session 5 (see Supplementary Table 1). Considering that two sessions with significant groups differences were observable before the introduction of the cognitive-interference task (FIG. 3A) and that volitional regulation during cognitive-interference did not correlate with training outcomes, it is unlikely that this difference could explain the other group differences found in the current study. Furthermore, because Theta and Alpha contribute to the Amyg-EFP model (FIG. 6) it is important to show that Amyg-EFP could be transferred to on-task demands. Such transferability might be critical for clinical translation in stress related disorders, as well as for preventive applications prior to exposure in prone populations (e.g. soldiers, fire fighters and policemen).

### Amyg-EFP-NF Training Outcome

Testing the effect of Amyg-EFP-NF on several domains and comparing this effect to control-NF and NoNF provides valuable insights into the current debate regarding the specificity of targeted signal modulation during NF to the targeted outcome³⁶. Relative to both controls, Amyg-EFP-NF resulted in a reduction in self-reports of alexithymia and performance improvements on an eStroop task (FIGS. 4A-4E), suggesting a change that is specific to Amyg-EFP-NF. This was particularly evident in alexithymia for which the reduction also correlated with Amyg-EFP signal regulation among Amyg-EFP-NF trainees only (FIG. 4E). Demonstrating a reduction in alexithymia following Amyg-EFP-NF is particularly interesting in light of the alleviated alexithymia scores observed by the NoNF, possibly due to the relatively stressful period during the first few weeks of military training³⁷. These results point to a possible stress inoculation effect of learning to down regulate an amygdala related neural signal. Considering previous research associating alexithymia with PTSD and combat related PTSD in particular³⁸, the current results may further indicate the clinical potential of Amyg-EFP-NF. This assertion is supported by the finding that only Amyg-EFP-NF led to reduced alexithymia among participants with moderate-severe baseline alexithymia (TAS-20≥51). Nevertheless, as expected from an a-priori healthy sample, less than a third exhibited moderate alexithymia and less than 5% exhibited severe alexithymia (TAS-20≥61). Further research with clinical populations exhibiting high alexithymia at baseline is needed to fully understand the relation between amygdala targeted NF and alexithymia, and whether such a relation interacts with the overall clinical prognosis.

In contrast to alexithymia, reduction in state-anxiety was observed following both Amyg-EFP-NF and control-NF with no correlations to either Amyg-EFP, A/T signal, nor to Amygdala-BOLD regulation in follow-up fMRI-NF. Together, these findings point to the reduction in state-anxiety as resulting from general NF training effects²³ that are not specific to Amyg-EFP signal reductions. Interestingly, while in the current work an effect of Amyg-EFP-NF on emotional conflict regulation in the eStroop task was demonstrated, in a previous work⁴ an influence on emotional adaptation was found ([ci]-[ii]). This discrepancy might be explained by the different designs and populations used in each study. In a previous work the pre- and post-NF measurements were conducted on the same day following a single session. It is possible that the relatively stressful period between the two measurements in the current study mediated the effect on emotional adaptation. Also, considering that no correlation was found between NF success and improved eStroop performance, future replication of this result is needed to corroborate this effect as an Amyg-EFP-NF specific process modification. Future studies should further assess the long-term sustainability of the effects demonstrated in the current study and whether Amyg-EFP-NF could reduce the likelihood of developing stress related psychopathology following traumatic exposure.

### Amyg-EFP-NF Target-Engagement

One of the goals in the current work was to test target engagement in the amygdala and its associated cortical connections. To test that an amygdala targeted fMRI-NF was performed approximately one month following the completion of Amyg-EFP-NF training. As expected, relative to NoNF, Amyg-EFP-NF resulted in a better ability to down-regulate amygdala BOLD using fMRI-NF (FIG. 5A). A similar result⁴ showing that one session of Amyg-EFP-NF resulted in improved amygdala BOLD down regulation compared to sham-NF was obtained. By conducting multiple sessions, the current study further showed that the learned skill of amygdala regulation can be sustained (longer than one month), and that one's best performance during training correlated with one's success on a follow-up fMRI-NF (FIG. 5B). This demonstration of transferability between EEG based repeated training to fMRI guided volitional regulation holds great promise in making region targeted NF clinically applicable. From a mechanistic perspective the PPI analysis showed that relative to NoNF, Amyg-EFP-NF resulted in higher amygdala-vmPFC functional connectivity (FIG. 5C), possibly suggesting an adaptive plasticity of a major path in the emotion regulation circuit³⁹. This result is consistent with converging evidence demonstrating that amygdala-vmPFC functional connectivity increases following amygdala targeted volitional regulation training^{10-13,40}. Together these findings demonstrate not only the plausibility of the amygdala as a target of volitional regulation but more so the adaptive effect that such regulation training could have on neural circuits central to emotion regulation.

Comparing post training fMRI-NF performance following Amyg-EFP-NF to NoNF only, and not control-NF, is a main limitation of the current study. As a reduction in state anxiety was observed both following Amyg-EFP-NF and control-NF it could be suggested that merely learning to control a brain signal may lead to reduced anxiety and better control over amygdala activity in fMRI-NF. As stated above however, no correlation between A/T modulation and reductions in state anxiety, nor between reductions in state anxiety and follow-up fMRI was found. Together with similar previous results obtained with simultaneous EEG/fMRI, these point to anxiety reduction as an unspecific effect of training with no evidence of a relation to volitional regulation of amygdala during fMRI-NF⁴¹. Future demonstrations of target engagement relative to an active control may be important. It could also be contended that a pre-training fMRI scan is essential to assert causality between Amyg-EFP-NF and amygdala volitional regulation during fMRI-NF. However, it should be noted that the population of the current study was highly homogeneous, consisting only of healthy males aged 18-24, all undergoing the same military training with the same daily schedule and nutrition.

### Conclusions

The current results suggest that learning to down regulate the amygdala via Amyg-EFP-NF strengthened amygdala-vmPFC connectivity and was specific to the cognitive processing of emotions (alexithymia and eStroop) but not necessarily to state anxiety. These findings are in line with recent perspectives of the amygdala as not only a ‘fear center’, as initially assumed⁴²⁻⁴⁵, but as also involved in the integration of introspective and sensory information allowing for higher order emotional processing^{2,46,47.} Demonstrating that this limbic mechanism can be modified via a scalable approach such as the EFP may facilitate clinical translation. Implementation of additional EFP models targeting different brain matrices, along with content specific interfaces, could further enhance the mechanistic specificity of the intervention, especially in context to specific disturbances such as PTSD, OCD or phobia.

### Validation Experiment Methods

NIH trial registration number: NCT02020265.
www(dot)clinicaltrials(dot)gov/ct2/show/NCT02020265

Participants: 180 healthy male Israeli Defense Forces (IDF) combat soldiers (aged 18-24) were recruited to the study during basic training and prior to operational deployment. Physiological, including neurological, health was pre-determined during military screening. Exclusion criteria consisted of an existing diagnosis of a mental disorder or use of psychoactive drugs, regarding which the participants were asked to report on prior to agreeing to enroll in the study. NF training and pre- and post- behavioral measurements took place at the military training base. Post-training fMRI scans were conducted at the Sagol Brain Institute, Wohl Institute for Advanced Imaging, Tel-Aviv Sourasky Medical Center. All participants gave written informed consent. The study was approved both by the Sourasky Medical Center and the IDF ethics review boards.

Procedure: Participants were randomly assigned to one of three conditions: (1) Amyg-EFP-NF (n=90) (2) control-NF (n=45) or (3) No-NF (NoNF; n=45). The Amyg-EFP-NF group were trained in down-regulation of the Amyg-EFP signal. The control-NF group were trained in down-regulation of Alpha (8-12 Hz) relative to Theta (4-7 Hz) and the NoNF group underwent no NF training. The assignment to control-NF and Amyg-EFP-NF was double blind. The training protocol (FIGS. 2A-2B) included 6 NF sessions within a period of 4 weeks (˜1-2 sessions per week). Before group assignment all participants answered the 20 item Toronto Alexithymia Scale (TAS-20), the State and Trait Anxiety Inventory (STAI) and conducted an emotional Stroop task.

Four participants (1 Amyg-EFP; 3 control-NF) requested not to participate in the NF training and were excluded. Seven additional participants (1 Amyg-EFP; 4 control-NF; 1 NoNF) could not participate due to a change in their military posting and were thus also excluded. The final analysis included 168 participants (88 Amyg-EFP; 38 control-NF; 43 NoNF). One month following training 60 participants (30 Amyg-EFP-NF; 30 NoNF) underwent post-training fMRI-NF. Due to technical difficulties four participants of the NoNF group could not complete the fMRI-NF scan. The final fMRI analysis included 56 participants.

Randomization and Blinding: Participants were randomly assigned to either the Amyg-EFP-NF, Control-NF or NoNF groups at a 2:1:1 ratio respectively. Randomization took place following completion of the pre-assessment phase using a custom-made software. The software further allowed for blinding between Amyg-EFP-NF and Control-NF by providing on-line feedback without revealing the source signal. Both participants and experimenters were blind to NF group allocation.

NF Training: NF was guided by the animated scenario interface previously developed by Cavazza et al.⁴⁸ and validated by Cohen et al.²⁶. The paradigm across the 6 sessions followed a similar block design, composed of 5 training cycles, each including 3 consecutive conditions: (a) watch (60 sec.), (b) regulate (60 sec.) and (c) washout (30 sec.). During watch participants were instructed to passively view the interface animation and were explained that at this time the animation was not influenced by their brain activity. During regulate participants were instructed to find the mental strategy that would cause the animated figures to sit down and lower their voices. Instructions were intentionally unspecific, allowing individuals to adopt the mental strategy that they subjectively found most efficient⁴⁹. During washout blocks participants were instructed to tap their thumb to their fingers according to a 3-digit number that appeared on the screen. Sessions 1-3 included an additional warmup conducted before NeuroFeedback Training consisting of 2 cycles. NF success at each session was measured as mean difference in the targeted signal power (Amyg-EFP or A/T) between all regulate and watch conditions conducted at that session. To facilitate learning sustainability, following NF training in sessions 4-6 participants also underwent a no-feedback trial^{26,30}. The no-feedback trial was introduced upon completion of the five NF cycles via the animated scenario, from session 4 onward. This trial consisted of one 60 sec. long watch block in which participants were instructed to passively view a fixation cross followed by 2 consecutives regulate blocks, on which participants were instructed to down regulate their targeted brain signal (either Amyg-EFP or A/T) while still viewing the same fixation cross. Individuals were instructed to use the same mental strategies that were successful in modulating the target signal in previous sessions. To further test whether participants could down-regulate the targeted brain activity while engaged in an additional cognitive task, upon completion of NF training in sessions 5-6 a “cognitive-interference” trial was conducted during which participants were instructed to down-regulate the relevant brain signal while conducting a simultaneous memory task. The interference task consisted of a single cycle, including one watch condition (60 sec) and one regulate condition (120 sec). While regulating the targeted signal participants were instructed to memorize as many details as possible from the animated scenario (positioning of different characters, clothing, objects etc.). After the completion of the NF trial (watch and regulation conditions) participants were asked to answer a 13-item multiple choice questionnaire. The emotional Stroop task: Participants viewed fearful or happy facial expressions with superimposed congruent or incongruent words (“happy”\“fear”) and were asked to identify the emotional expression while ignoring the words that appeared. The emotional Stroop task provides a measure of ‘general conflict regulation’ measured by the difference in response times between congruent and incongruent stimuli and of ‘Emotional conflict adaptation’ measured by the difference in response times between two consecutive incongruent stimuli [ii] and incongruent stimulus following congruent stimulus [ci] (adaptation =[ii][ci])²².

Self-report questionnaires: Alexithymia was measured using the Hebrew version of the 20 item Toronto Alexithymia Scale (TAS), previously tested for reliability and factorial validity⁵⁰. TAS-20 measures difficulties in expressing and identifying emotions²¹, a tendency previously demonstrated to correlate with stress vulnerability^{28,29}. The overall alexithymia score comprises three sub-scores: (a) difficulty identifying feelings (IDF), (b) difficulty describing feelings (DDF) and (c) externally oriented thinking (EOT).

State anxiety was measured using the previously validated Hebrew version of the State Trait Anxiety Inventory (STAI)⁵¹. STAI²⁰ consists of two 20 item inventories measuring state and trait anxiety.

The Amyg-EFP model: The Amyg-EFP model was previously developed by our lab to enable the prediction of localized activity in the amygdala using EEG only^{5,6}. This was done by applying machine learning algorithms on EEG data acquired simultaneously with fMRI. The procedure resulted in a Time-Delay×Frequency×weight coefficient matrix. EEG data recorded from electrode Pz at a given time-point are multiplied by the coefficient matrix to produce the predicted amygdala fMRI-BOLD activity. Keynan et al.,⁴ validated the reliability of the Amyg-EFP in predicting amygdala BOLD activity by conducting simultaneous EEG-fMRI recordings using a new sample not originally used to develop the model.

EEG data acquisition and online processing: EEG data were acquired using the V-Amp™ EEG amplifier (Brain Products™, Munich Germany) and the BrainCap™ electrode cap with sintered Ag/AgCI ring electrodes providing 16 EEG channels, 1 ECG channel, and 1 EOG channel (Falk MinowServices™, Herrsching-Breitburnn, Germany). The electrodes were positioned according to the standard 10/20 system. The reference electrode was between Fz and Cz. Raw EEG was sampled at 250 Hz and recorded using the Brain Vision Recorder software (Brain Products).

On line calculation of Amyg-EFP and A/T power: Online EEG processing was conducted via the RecView software (Brain Products). RecView makes it possible to remove cardio-ballistic artifacts from the EEG data in real time using a built-in automated implementation of the average artifact subtraction method⁵². Amyg-EFP data were collected from electrode Pz and A/T ratio was extracted from electrodes 01, Oz and 02. RecView™ was custom modified to enable export of the corrected EEG data in real time through a TCP/IP socket. Preprocessing algorithm and signal (Amyg-EFP or A/T) calculation models were compiled from Matlab R2009b™ to Microsoft. NET™ in order to be executed within the Brain Vision RecView™ EEG Recorder system. Data were then transferred to a MATLAB.NET compiled DLL that calculated the value of the targeted signal power every 3 seconds.

Animated Scenario Feedback Generation: The neurofeedback interface included a virtual hospital waiting room whose visual setting constitutes a metaphor for arousal within a realistic context. Characters waiting in the room exist in a resting state (waiting seated) or agitated state (protesting at the counter) and the overall level of agitation depends on the ratio between these two states. This mechanism ensures smooth visual transitions through an individual characters' change of state and as a result the room as a whole may become either more agitated or more relaxed by the user (FIG. 2B; Supplementary Video²⁶). The ratio between characters sitting down and protesting at the counter is considered to be a two-state Boltzmann distribution⁴⁸, whose evolution is driven by a “virtual temperature” whose value is derived from the momentary value of the targeted signal power (Amyg-EFP or A/T). The scenario uses the probability (p value) of a momentary signal value during regulate to be sampled under the previous watch distribution. This p value is used to determine the probability of virtual characters to be moving in the virtual room, with the character distribution updated accordingly. A matching soundtrack recorded inside a real hospital complements the system output. Three alternative soundtracks with different agitation levels were produced and switched according to the signal value. During the watch condition 75% of the characters congregate at the front desk while expressing their frustration through body and verbal language. The system is implemented using the Unreal Development Kit (UDK™) game engine, which controls relevant animations (walking, sitting, standing, protesting), as well as their transitions for individual characters.

Statistical Analysis: Statistical analysis was conducted using IBM SPSS Statistics 20™, and MATLAB R2017b. NF Success in each session was measured as the mean difference in the targeted signal power (A/T or Amyg-EFP) during regulate relative to watch^{4,26}. The mean result of each group was analyzed using a repeated measures ANOVA with session (1-6) and group (Amyg-EFP-NF vs control-NF) as factors. Behavioral measures were each assessed with a separate repeated measures ANOVA with group (Amyg-EFP-NF, control-NF and NoNF) and time (pre- vs post-training) as factors. Unless specified otherwise, all reported p values are two-tailed. One-tailed tests were used only when a one-sided a-priori hypothesis existed. Data distribution was assumed to be normal, but this was not formally tested. Box plots showing data distribution (individual data points) for all variables are available in the supplementary information. Sphericity assumptions were tested using Box's test of equality of covariance matrices and Levene's test for equality of variances. Where sphericity assumption was violated, corrected statistics and p values were used.

Missing Data: To control for bias⁵³, missing data were imputed using multiple data imputation (predictive mean matching) with 5 iterations and was treated as missing at random. To account for the added uncertainty a repeated measures ANOVA was conducted following van Ginkel & Kroonenberg⁵⁴ correcting variances and degrees of freedom. Between and within groups simple effects were tested using built in SPSS procedure for t-test on multiply imputed data, accounting for added uncertainty.

Power analysis: Sample size calculation was based on behavioral results (emotional Stroop) from Keynan et al.,¹². The effect size of the group by time (pre- vs post-NF) interaction in Keynan et al., was relatively large (η²=0.19). Power analysis suggested that to allow detection (alpha=0.05) of a more conservative effect (η²=0.09), with at least 80% power in a 3 by 2 design, a total sample of 150 participants is required. Considering the expectation of an 85% retention rate we recruited 180 participants.

Post-training fMRI-NF: To test for target engagement in the amygdala, one month following training participants came to the Sagol Brain Institute and underwent amygdala targeted fMRI-NF. To further allow for the testing of learning transferability between contexts, and to refute the possibility that observed group difference are merely a result of familiarity with the animated scenario, the fMRI-NF paradigm was of a similar block design as in the training period but utilized different and unfamiliar visual feedback¹². This visual interface consisted of a 2D unimodal flash-based graphic interface with an animated figure standing on a skateboard, skating down a rural road. The participant's goal was to lower the speed of the moving skateboard which is determined by amygdala beta (mean parameter estimates) weighted activity. During watch the skateboard moved at a constant pre-set speed of 90km/h. During regulate the skateboard's speed was set in accordance to the momentary amygdala beta weighted activity ranging between 50-130 km/h. To avoid new learning, the fMRI-NF paradigm consisted of 2 cycles¹².

Real-time calculation of amygdala activity and visual feedback generation: The visual feedback is generated in a mathematically identical manner to the animated scenario, only using amygdala beta weighted activity instead of Amyg-EFP power. Momentary beta weights of the pre-defined amygdala region of interest (ROI) were extracted on-line using Turbo Brain voyager 3.0™ (Brain Innovation, Maastricht, Netherlands). The beta weights were then transferred to MATLAB™ which in turn set the speed of the moving skate board. The amygdala ROI was defined according to the Talairach coordinates of the amygdala functional cluster used for the initial Amyg-EFP model development¹¹ (x=20, y=−5, z=−17; 3 mm Gaussian sphere).

fMRI data acquisition: Structural and functional scans were performed in a 3.0 Tesla Siemens MRI system (MAGNETOM Prisma, Germany) using a twenty-channel head coil. To allow high-resolution structural images a T1-weighted 3D Sagittal MPRAGE pulse sequence (TR/TE=1860/2.74 ms, flip angle=8°, pixel size=1×1 mm, FOV=256×256 mm) was used. Functional whole-brain scans were performed in an interleaved top-to-bottom order, using a T2*-weighted gradient echo planar imaging pulse sequence (TR/TE=3000/35 ms, flip angle=90°, pixel size=1.56 mm, FOV=200×200 mm, slice thickness=3 mm, 44 slices per volume). A sample of 13 participants were scanned with a GE 3T Signa scanner using the same parameters only with 39 slices per volume. No differences were found between scanners on the measured ROIs.

fMRI data preprocessing: Preprocessing and statistical analysis were performed using BrainVoyager QX version 2.8 (Brain Innovation, Maastricht, Netherlands). Slice scan time correction was performed using cubic-spline interpolation. Head motions were corrected by rigid body transformations, using three translations and three rotation parameters and the first image served as a reference volume. Trilinear interpolation was applied to detect head motions and sinc interpolation was used to correct them. The temporal smoothing process included linear trend removal and usage of a high-pass filter of 1/128 Hz. Functional maps were manually co-registered to corresponding structural maps and together they were incorporated into three-dimensional datasets through trilinear interpolation. The complete dataset was transformed into Talairach space and spatially smoothed with an isotropic 8 mm full width at half maximum (FWHM) Gaussian kernel.

Amygdala region of interest (ROI) analysis: Using a random-effects general linear model (GLM), beta values were extracted for all the voxels in the amygdala ROI targeted during fMRI-NF. The model included 3 regressors for each condition (watch, regulate and washout). Regressors were convolved with a canonical hemodynamic response function. Additional nuisance regressors included the head-movement realignment parameters. A two-way repeated measures ANOVA was then conducted with the amygdala beta values as a dependent variable and group (Amyg-EFP-NF vs NoNF) and condition (watch vs regulate) as factors.

Amygdala whole brain psycho-physiological interaction (PPI): Group (Amyg-EFP-NF>NoNF) differences in functional connectivity during watch and regulate were examined using an in-house generalized psychophysiological interaction (PPI) analysis tool, previously implemented in our lab for Brainvoyager⁵⁵. A whole-brain psycho-physiological interaction (PPI) random effects GLM analysis was conducted, using the psychological variables (the original regressors of the fMRI-NF paradigm) and the physiological variable (the activity time course of the seed amygdala ROI) as regressors.

### References for “Exemplary Validation Experiment”

1. Johnston, S. J., Boehm, S. G., Healy, D., Goebel, R. & Linden, D. E. J. Neurofeedback: A promising tool for the self-regulation of emotion networks. NeuroImage 49, 1066-1072 (2010).

2. Pessoa, L. & Adolphs, R. Emotion processing and the amygdala: from a ‘low road’ to ‘many roads’ of evaluating biological significance. Nat. Rev. Neurosci. 11, 773-783 (2010).

3. Birbaumer, N., Ruiz, S. & Sitaram, R. Learned regulation of brain metabolism. Trends Cogn. Sci. 17, 295-302 (2013).

4. Keynan, J. N. et al. Limbic Activity Modulation Guided by Functional Magnetic Resonance Imaging—Inspired Electroencephalography Improves Implicit Emotion Regulation. Biol. Psychiatry 80, 490-496 (2016).

5. Meir-Hasson, Y., Kinreich, S., Podlipsky, I., Hendler, T. & Intrator, N. An EEG Finger-Print of fMRI deep regional activation. NeuroImage 102, 128-141 (2014).

6. Meir-Hasson, Y. et al. One-Class FMRI-Inspired EEG Model for Self-Regulation Training. PLOS ONE 11, e0154968 (2016).

7. Johnstone, T., Reekum, C. M. van, Urry, H. L., Kalin, N. H. & Davidson, R. J. Failure to Regulate: Counterproductive Recruitment of Top-Down Prefrontal-Subcortical Circuitry in Major Depression. J. Neurosci. 27, 8877-8884 (2007).

8. Gross, J. J. Emotion Regulation: Current Status and Future Prospects. Psychol. Inq. 26, 1-26 (2015).

9. Admon, R. et al. Human vulnerability to stress depends on amygdala' s predisposition and hippocampal plasticity. Proc. Natl. Acad. Sci. 106, 14120-14125 (2009).

10. Paret, C. et al. fMRI neurofeedback of amygdala response to aversive stimuli enhances prefrontal—limbic brain connectivity. NeuroImage 125, 182-188 (2016).

11. Nicholson, A. A. et al. The neurobiology of emotion regulation in posttraumatic stress disorder: Amygdala downregulation via real-time fMRI neurofeedback. Hum. Brain Mapp. 38, 541-560(2017).

12. Zotev, V., Phillips, R., Young, K. D., Drevets, W. C. & Bodurka, J. Prefrontal Control of the Amygdala during Real-Time fMRI Neurofeedback Training of Emotion Regulation. PLOS ONE 8, e79184 (2013).

13. Paret, C. et al. Alterations of amygdala-prefrontal connectivity with real-time fMRI neurofeedback in BPD patients. Soc. Cogn. Affect. Neurosci. 11, 952-960 (2016).

14. Marxen, M. et al. Amygdala Regulation Following fMRI-Neurofeedback without Instructed Strategies. Front. Hum. Neurosci. 10, (2016).

15. Young, K. D. et al. Randomized Clinical Trial of Real-Time fMRI Amygdala Neurofeedback for Major Depressive Disorder: Effects on Symptoms and Autobiographical Memory Recall. Am. J. Psychiatry 174, 748-755 (2017).

16. Thibault, R. T., Lifshitz, M., Birbaumer, N. & Raz, A. Neurofeedback, Self-Regulation, and Brain Imaging: Clinical Science and Fad in the Service of Mental Disorders. Psychother. Psychosom. 84, 193-207 (2015).

17. Hendler, Intrator, Klovatch, Kinreich & Hasson. Method and system for use in analyzing neural activity in a subject's brain. United States Provisional Patent Application No. US20140148657 A1, WO2012104853 A3, EP2670299 A2. (2011).

18. Gold, M. A. Cadet basic training: An ethnographic study of stress and coping. Mil. Med. Bethesda 165, 147-52 (2000).

19. Larson, G. E. Physical symptoms as indicators of depression and enxiety. Mil. Med. Bethesda 166, 796-9 (2001).

20. Spielberger, C. D. & Spielberger, C. D. State-Trait Anxiety Inventory, State-Trait Anxiety Inventory. in Corsini Encyclopedia of Psychology, Corsini Encyclopedia of Psychology (John Wiley & Sons, Inc., John Wiley & Sons, Inc., 2010). doi:10.1002/9780470479216.corpsy0943, 10.1002/9780470479216.corpsy0943

21. Taylor, G. J., Bagby, R. M. & Parker, J. D. A. Dissorders of affect regulation. (Cambridge, RU: Cambridge University Press, 1997).

22. Etkin, A., Prater, K. E., Hoeft, F., Menon, V. & Schatzberg, A. F. Failure of Anterior Cingulate Activation and Connectivity With the Amygdala During Implicit Regulation of Emotional Processing in Generalized Anxiety Disorder. Am. J. Psychiatry 167, 545-554 (2010).

23. Sitaram, R. et al. Closed-loop brain training: the science of neurofeedback. Nat. Rev. Neurosci. 18, 86-100 (2017).

24. Alegria, A. A. et al. Real-time fMRI neurofeedback in adolescents with attention deficit hyperactivity disorder. Hum. Brain Mapp. 38, 3190-3209 (2017).

25. Gruzelier, J. H. EEG-neurofeedback for optimizing performance. III: A review of methodological and theoretical considerations. Neurosci. Biobehay. Rev. 44, 159-182 (2014).

26. Cohen, A. et al. Multi-modal Virtual Scenario Enhances Neurofeedback Learning. Front. Robot. AI 3, (2016).

27. Etkin, A., Egner, T., Peraza, D. M., Kandel, E. R. & Hirsch, J. Resolving Emotional Conflict: A Role for the Rostral Anterior Cingulate Cortex in Modulating Activity in the Amygdala. Neuron 51, 871-882 (2006).

28. Durham, C. N. Posttraumatic stress disorder and resilience in Iraq and Afghanistan veterans: The mediator roles of masculine gender role stress and alexithymia. (New Mexico State University, 2016).

29. FREWEN, P. A., PAIN, C., DOZOIS, D. J. A. & LANIUS, R. A. Alexithymia in PTSD. Ann. N. E Acad. Sci. 1071, 397-400 (2006).

30. Thibault, R. T., Lifshitz, M. & Raz, A. The self-regulating brain and neurofeedback: Experimental science and clinical promise. Cortex 74, 247-261 (2016).

31. Franz, M. et al. Alexithymia in the German general population. Soc. Psychiatry Psychiatr. Epidemiol. 43, 54-62 (2008).

32. Salminen, J. K., Saarijärvi, S., Äärelä, E., Toikka, T. & Kauhanen, J. Prevalence of alexithymia and its association with sociodemographic variables in the general population of finland. J. Psychosom. Res. 46, 75-82 (1999).

33. Lindholm, T., Lehtinen, V., Hyyppa, M. T. & Puukka, P. Alexithymic features in relation to the dexamethasone suppression test in a Finnish population sample. Am. J. Psychiatry 147, 1216-1219 (1990).

34. Linden, D. E. J. et al. Real-Time Self-Regulation of Emotion Networks in Patients with Depression. PLOS ONE 7, e38115 (2012).

35. Censor, N., Sagi, D. & Cohen, L. G. Common mechanisms of human perceptual and motor learning. Nat. Rev. Neurosci. 13, 658-664 (2012).

36. Thibault, R. T., Lifshitz, M. & Raz, A. Neurofeedback or neuroplacebo? Brain 140, 862-864 (2017).

37. De Vente, W., Kamphuis, J. H. & Emmelkamp, P. M. G. Alexithymia, risk factor or consequence of work-related stress? Psychother. Psychosom. 75, 304-311 (2006).

38. Frewen, P. A., Dozois, D. J. A., Neufeld, R. W. J. & Lanius, R. A. Meta-analysis of alexithymia in posttraumatic stress disorder. J. Trauma. Stress 21, 243-246 (2008).

39. Etkin, A., Egner, T. & Kalisch, R. Emotional processing in anterior cingulate and medial prefrontal cortex. Trends Cogn. Sci. 15, 85-93 (2011).

40. Zotev, V. et al. Real-time fMRI neurofeedback training of the amygdala activity with simultaneous EEG in veterans with combat-related PTSD. NeuroImage Clin. 19, 106-121 (2018).

41. Kinreich, S., Podlipsky, I., Intrator, N. & Hendler, T. Categorized EEG neurofeedback performance unveils simultaneous fMRI deep brain activation. Mach. Learn. Interpret. Neuroimaging 108-115 (2012).

42. Ohman, A. & Mineka, S. Fears, phobias, and preparedness: toward an evolved module of fear and fear learning. Psychol. Rev. 108, 483 (2001).

43. Dolan, R. J. & Vuilleumier, P. Amygdala Automaticity in Emotional Processing. Ann. N. Y. Acad. Sci. 985, 348-355 (2006).

44. Morris, J. S., Ohman, A. & Dolan, R. J. A subcortical pathway to the right amygdala mediating “unseen” fear. Proc. Natl. Acad. Sci. U.S.A. 96, 1680-1685 (1999).

45. LeDoux, J. The Emotional Brain, Fear, and the Amygdala. Cell. Mol. Neurobiol. 23, 727-738 (2003).

46. Haber, S. N. & Knutson, B. The Reward Circuit: Linking Primate Anatomy and Human Imaging. Neuropsychopharmacology 35, 4-26 (2010).

47. Barrett, L. F. & Simmons, W. K. Interoceptive predictions in the brain. Nat. Rev. Neurosci. 16, 419 (2015).

48. Cavazza, M. et al. Towards Emotional Regulation Through Neurofeedback. in Proceedings of the 5th Augmented Human International Conference 42:1-42:8 (ACM, 2014). doi: 10.1145/2582051.2582093

49. Shibata, K., Watanabe, T., Sasaki, Y. & Kawato, M. Perceptual Learning Incepted by Decoded fMRI Neurofeedback Without Stimulus Presentation. Science 334, 1413-1415 (2011).

50. Taylor, G. J., Bagby, R. M. & Parker, J. D. A. The 20-Item Toronto Alexithymia Scale: IV. Reliability and factorial validity in different languages and cultures. J. Psychosom. Res. 55, 277-283 (2003).

51. Teichman, Y. & Melnick, H. The Hebrew manual for the state-trait anxiety inventory. Ramot Isr. Tel-Aviv Univ. (1980).

52. Allen, P. J., Polizzi, G., Krakow, K., Fish, D. R. & Lemieux, L. Identification of EEG Events in the MR Scanner: The Problem of Pulse Artifact and a Method for Its Subtraction. NeuroImage 8, 229-239 (1998).

53. Sterne, J. A. C. et al. Multiple imputation for missing data in epidemiological and clinical research: potential and pitfalls. BMJ 338, b2393 (2009).

54. Ginkel, J. R. van & Kroonenberg, P. M. Analysis of Variance of Multiply Imputed Data. Multivar. Behay. Res. 49, 78-91 (2014).

55. Gilam, G. et al. Neural substrates underlying the tendency to accept anger-infused ultimatum offers during dynamic social interactions. NeuroImage 120, 400-411 (2015).

### Supplementary Methods and Results for the Exemplary Validation Experiment

| | NeuroFeedback | | Cognitive- |
|---|---|---|---|
| | Training | No-Feedback | Interfernce |
| | (5 Cycels, 12:30 min.) | (2 Cyces, 3 min.) | (1 Cycle, 2 min.) |
| Session 1¹ | √ | | |
| Session 2¹ | √ | | |
| Session 3¹ | √ | | |
| Session 4 | √ | √ | |
| Session 5 | √ | √ | √ |
| Session 6 | √ | √ | √ |

Supplementary Table 1: Order and type of NF tasks conducted at each session. NF training included 5 cycles (FIG. 2B) and was performed in all sessions. During the No-Feedback condition participants were instructed to down regulate the recorded brain signal (Amyg-EFP or A/T ratio) in the absence of online feedback. In the cognitive-interference condition participants were instructed to down regulate the recorded brain signal while simultaneously memorizing details of the animated 3D scenario (see method).
¹Sessions 1-3 included an additional warmup conducted before NeuroFeedback Training consisting of 2 cycles.

| | |
|---|---|
| Amyg-EFP-NF | Control-NF |

| | | | Mean CI (95%) | | | | Mean CI (95%) | |
|---|---|---|---|---|---|---|---|---|
| A | Mean | sd | Lower | Upper | Mean | sd | Lower | Upper |
| Session 1 | −0.05 | 0.13 | −0.08 | −0.03 | −0.01 | 0.14 | −0.06 | 0.03 |
| Session 2 | −0.09 | 0.13 | −0.12 | −0.07 | −0.04 | 0.08 | −0.08 | −0.01 |
| Session 3 | −0.09 | 0.15 | −0.13 | −0.06 | −0.06 | 0.15 | −0.11 | 0.01 |
| Session 4 | −0.10 | 0.17 | −0.14 | −0.07 | −0.02 | 0.16 | −0.07 | 0.03 |
| Session 5 | −0.12 | 0.18 | −0.15 | −0.08 | −0.03 | 0.13 | −0.08 | 0.03 |
| Session 6 | −0.16 | 0.20 | −0.20 | −0.12 | 0.01 | 0.18 | −0.05 | 0.07 |

Between Group Comparison (Amyg-EFP-NT-Control-NF)

| | | | | | | Effect Size CI (95%) | |
|---|---|---|---|---|---|---|---|
| B | Mean | se | t(124) | p | d | Lower | Upper |
| Session 1 | −0.04 | 0.03 | 1.37 | =0.173 | 0.27 | −0.12 | 0.65 |
| Session 2 | −0.05 | 0.03 | 1.66 | =0.107 | 0.32 | −0.06 | 0.70 |
| Session 3 | −0.04 | 0.03 | 1.04 | =0.298 | 0.20 | −0.18 | 0.58 |
| Session 4 | −0.09 | 0.04 | 2.46 | =0.014 | 0.48 | 0.09 | 0.86 |
| Session 5 | −0.09 | 0.04 | 2.36 | =0.020 | 0.46 | 0.07 | 0.84 |
| Session 6 | −0.17 | 0.04 | 3.87 | <0.001 | 0.75 | 0.36 | 1.14 |

Supplementary Table 2: Amyg-EFP signal modulations (regulate-watch) of each group at each session. (A) Means, Standard Deviations (sd), and CIs of Amyg-EFP signal down regulation (Regulate-Watch) of each group at each session. (B) Means, standard errors (se), t statistics, p values effect size estimations (Cohen's d) and 95% CIs of a between groups comparison conducted for each session. One can see that session 4-6 show significant group differences with enlarging effect sizes.

| | Amyg-EFP-NF | | | | | | |
|---|---|---|---|---|---|---|---|
| | | | | | | Effect Size | |
| | | | | | | CI (95%) | |
| | Mean | sd | t(87) | p | d | Lower | Upper |
| Session 1 vs 2 | −0.04 | 0.19 | 1.89 | =0.058 | 0.20 | −0.01 | 0.41 |
| Session 1 vs 3 | −0.04 | 0.23 | 1.63 | =0.105 | 0.17 | −0.04 | 0.38 |
| Session 1 vs 4 | −0.05 | 0.25 | 1.95 | =0.052 | 0.21 | 0.00 | 0.42 |
| Session 1 vs 5 | −0.07 | 0.30 | 2.05 | =0.047 | 0.22 | 0.01 | 0.43 |
| Session 1 vs 6 | −0.11 | 0.25 | 4.06 | <0.001 | 0.43 | 0.21 | 0.65 |
| | Control-NF | | | | | | |
| | | | | | | Effect Size | |
| | | | | | | CI (95%) | |
| | Mean | se | t(37) | p | d | Lower | Upper |
| Session 1 vs 2 | −0.03 | 0.24 | 0.70 | =0.494 | 0.11 | −0.21 | 0.43 |
| Session 1 vs 3 | −0.04 | 0.19 | 1.42 | =0.156 | 0.23 | −0.09 | 0.55 |
| Session 1 vs 4 | −0.01 | 0.22 | 0.14 | =0.892 | 0.02 | −0.30 | 0.34 |
| Session 1 vs 5 | −0.01 | 0.21 | 0.43 | =0.671 | 0.07 | −0.25 | 0.39 |
| Session 1 vs 6 | 0.02 | 0.22 | 0.63 | =0.527 | 0.10 | −0.22 | 0.42 |

Supplementary Table 3: Improvement in Amyg-EFP signal modulations of each group relative to the first session. Mean, Sd, t statistic, p value, effect size estimate (Cohen's d) and 95% CI, of within group comparisons of Amyg-EFP signal modulation (regulate-watch) between each session (2-6) and the first session.

| | Control-NF (A/T ratio) | | | | | | | | |
|---|---|---|---|---|---|---|---|---|---|
| | | | | Delta vs Session 1 | | | | Effect Size CI (95%) | |
| | Mean | Sd | Mean | Sd | t(37) | p | d | Lower | Upper |
| Session 1 | 0.002 | 0.07 | | | | | | | |
| Session 2 | 0.005 | 0.10 | 0.003 | 0.09 | 0.19 | 0.853 | 0.03 | −0.29 | 0.35 |
| Session 3 | 0.010 | 0.08 | 0.008 | 0.09 | 0.55 | 0.586 | 0.09 | −0.23 | 0.41 |
| Session 4 | −0.011 | 0.10 | −0.014 | 0.13 | 0.67 | 0.505 | 0.11 | −0.21 | 0.43 |
| Session 5 | −0.040 | 0.09 | −0.042 | 0.12 | 2.25 | 0.025 | 0.36 | 0.03 | 0.69 |
| Session 6 | −0.043 | 0.10 | −0.045 | 0.13 | 2.22 | 0.026 | 0.36 | 0.03 | 0.69 |

Supplementary Table 4: Control-NF A/T ratio signal modulation at each session and improvement relative to the first session. The left sided Means and Sds are of the average performance (relate-watch) at each session. The following columns report, Mean, Sd, t statistic, p value, effect estimate (Cohen's d) and 95% CI, of within group comparisons of A/T signal modulations (regualte-watch) between each session (2-6) and the first session.

| | Amyg-EFP-NT | | | | Control-NF | | | |
|---|---|---|---|---|---|---|---|---|
| | | | Mean | | | | Mean | |
| | | | CI (95%) | | | | CI (95%) | |
| | Mean | sd | Lower | Upper | Mean | sd | Lower | Upper |
| Session | −0.05 | 0.09 | −0.07 | −0.03 | −0.04 | 0.10 | −0.07 | −0.01 |
| 1 | | | | | | | | |
| Session | −0.08 | 0.09 | −0.09 | −0.06 | −0.05 | 0.07 | −0.07 | −0.02 |
| 2 | | | | | | | | |
| Session | −0.09 | 0.08 | −0.10 | −0.07 | −0.04 | 0.09 | −0.07 | −0.02 |
| 3 | | | | | | | | |
| Session | −0.09 | 0.12 | −0.12 | −0.06 | −0.01 | 0.15 | −0.05 | 0.04 |
| 4 | | | | | | | | |
| Session | −0.11 | 0.14 | −0.13 | −0.08 | −0.03 | 0.12 | −0.08 | 0.01 |
| 5 | | | | | | | | |
| Session | −0.12 | 0.14 | −0.14 | −0.09 | 0.01 | 0.12 | −0.04 | 0.05 |
| 6 | | | | | | | | |

Supplementary Table 5: Statistics of Amyg-EFP signal modulations following outlier removal. The table reposts means, sds, CIs of Amyg-EFP signal reductions (regulate-watch) of each group in each session.

| | Pre-Training | | | | Post-Training | | | |
|---|---|---|---|---|---|---|---|---|
| | | | Mean CI (95%) | | | | Mean CI (95%) | |
| | Mean | sd | Lower | Upper | Mean | sd | Lower | Upper |
| | Amyg-EFP-NF | | | | | | | |
| e-Conflict Regulation | 42.23 | 29.92 | 36.73 | 49.73 | 33.26 | 27.11 | 27.76 | 38.75 |
| (Incong.-Cong.) | | | | | | | | |
| e-Conflict Adaptation | −2.37 | 45.74 | −11.34 | 6.61 | 5.95 | 31.48 | −1.21 | 13.11 |
| (ii-ci) | | | | | | | | |
| Alexithymia (TAS-20) | 42.95 | 11.22 | 40.62 | 45.29 | 39.58 | 11.63 | 36.87 | 42.29 |
| State Anxiety (STAI) | 31.46 | 9.55 | 29.48 | 33.45 | 29.20 | 7.76 | 27.51 | 30.90 |
| | Control-NF | | | | | | | |
| e-Conflict Regulation | 37.31 | 35.01 | 27.41 | 47.20 | 41.47 | 27.61 | 33.10 | 49.83 |
| (Incong.-Cong.) | | | | | | | | |
| e-Conflict Adaptation | −0.97 | 44.35 | −14.62 | 12.68 | −6.77 | 38.92 | −17.66 | 4.12 |
| (ii-ci) | | | | | | | | |
| Alexithymia (TAS-20) | 41.99 | 10.85 | 38.44 | 45.54 | 42.00 | 13.25 | 37.88 | 46.12 |
| State Anxiety (STAI) | 33.47 | 10.29 | 30.46 | 36.49 | 30.23 | 7.64 | 27.64 | 32.81 |
| | NoNF | | | | | | | |
| e-Conflict Regulation | 41.01 | 29.38 | 37.71 | 50.31 | 51.28 | 22.89 | 43.42 | 59.15 |
| (Incong.-Cong.) | | | | | | | | |
| e-Conflict Adaptation | −4.06 | 33.35 | −16.90 | 8.77 | −1.02 | 34.39 | −11.26 | 9.22 |
| (ii-ci) | | | | | | | | |
| Alexithymia (TAS-20) | 42.00 | 11.02 | 38.67 | 45.34 | 48.11 | 13.57 | 44.24 | 51.98 |
| State Anxiety (STAI) | 32.81 | 8.92 | 29.97 | 35.65 | 32.20 | 9.00 | 29.77 | 34.62 |

Supplementary Table 6: Behavioral outcome measures. The table reports means, sds and CIs of each group at each time point.

### Supplementary Methods

Control condition justification: A control condition should account for three of the global processes that are induced by NF without targeting the mechanism of interest. These main processes are (a) reward: a feedback cue indicating success or unsuccess; (b) control: control on a mental state and brain signal; and (c) learning: the consolidation of associations between an applied mental strategy and its outcome via operant learning. In fMRI-NF for example a control condition that deals with such general processes should consist of feedback from a different region¹⁻³. A yoked sham control on the other hand, would account for the reward aspect but would not generate contingent learning. Indeed, in a previous study⁴ a yoked sham control was used, in which participants received feedback derived from the Amyg- EFP signal of a different participant. Following training when given the opportunity to regulate via veritable feedback in a follow-up fMRI-NF session, participants who trained via sham-NF showed an impaired ability to volitionally regulate the amygdala. Thus, the yoked sham was actually an active control of incorrect learning that could bias the results. Similar results were obtained recently in a study testing the placebo control using NF in a systematic manner⁵. Furthermore, when conducting repeated sessions, as in the current study, participants may notice the lack of contingency between the feedback and their mental effort. Additional options could include random feedback that also lacks contingency, training regulation in the inverse direction (amygdala upregulation) that may have undesired influences and mental rehearsal without NF which disables blinding.

In the validation experiment, and in some embodiments of the invention an Alpha/Theta probe⁶ is used to control for these general processes, which is the EEG equivalent of a “different region” approach. Moreover, since theta and alpha contribute to the Amyg-EFP, a specificity of the Amyg-EFP to limbic processing was demonstrated; not only using a correlative approach as done previously⁴ but by also causally showing amygdala related behavioral changes following Amyg-EFP-NF in contrast to A/T-EEG-NF alone.

According to previous studies of A/T-EEG-NF (see Gruzlier et al.^{6,7} for review) the underlying assumption was that A/T-EEG-NF mainly targets general arousal brain networks. An assumption also supported by the concurrent fMRI\EEG study, demonstrating the fMRI correlates of successful A/T modulation⁸.

Selection of number of sessions: Successful amygdala volitional regulation was previously shown in fMRI-NF studies following relatively few sessions (up to 3)^{2,9,10}. A previous study similarly demonstrated improved amygdala BOLD regulation following a single sessions of Amyg-EFP-NF⁴. While conventional EEG studies commonly apply at-least 10 sessions, learning A/T regulation was observed with healthy participants after less than 6 sessions^{11,12,8}. Considering the intensive military training the participants underwent, in addition to the reported feasibility of the effect following relatively few NF sessions, 6 sessions were administered.

As the results show, learning to control the targeted signal was observed in Amyg-EFP-NF following 4 sessions (FIGS. 4A-4E) and following session 5 in the control-NF group, for example as shown in FIGS. 7A and 7B. FIG. 7A show results obtained for the Amyg-EFP-NF group and Fig. B show results obtained for the Control-NF group. In FIGS. 7A and 7B the mean and median are marked respectively by an X and a line inside each box., and Whisker lines represent 1.5× interquartile range.

Nevertheless, as stated in the discussion (lines 340-350) the current findings suggest that learning was not exhausted after six session and that the optimal number of sessions should be systematically investigated in future studies.

Correlating NF success and outcome measures: To correlate individual NF success and training outcome, an index that captures individual learning potential was developed while taking in to account that different individuals show differently shaped learning curves¹³. The average performance across six sessions is influenced by the first session in which participants have yet to be trained. The delta between the first and last session assumes that each individual will reach the best performance at the last session. A coefficient of the slop also assumes a similarly shaped learning curve between individuals. The best performance out of 2 to 10 sessions or any intermediate, smaller or larger number of sessions, for example six sessions was used as index of learning potential making less a-priori assumptions.

Protocol: Amygdala-EFP NF Guided Resilience Training Interfaced with Animated Scenario.

1. Overview: The training protocol is composed of six NF meetings each consisting serval training trials as detailed in table 1 below.

At the first session the trainee is explained that the porous of the training is to enhance stress resilience by acquiring volitional control of amygdala activity. It is explained that the participant will view a simulation of an agitated hospital waiting room of which agitation level is controlled by the participant's amygdala activation level. The NF trainees are instructed to find the mental state that corresponds to an ease in the unrest level of the animated scenario (i.e. causes people to seat down calmly). Instructions are intentionally unspecific, allowing individuals to adopt the mental strategy that they subjectively find most efficient.

2. NF trial types: A typical NF (FIGS. 2A and 2B) trial is generally consisted of 1-5 cycles each including 3 consecutive conditions (‘Watch’, ‘Regulate’ and ‘Wash Out’) varying in duration detailed bellow and in table 1. During watch participants are instructed to passively view the scenario which is fixed on 75% agitation level. It is explained that at this time the participant's amygdala activity level does not affect the scenario and that the participant should not employ any mental strategy. During regulate the participant is instructed to find the mental strategy that corresponds an appeasement in the scenario unrest level. During washout the participant taps his thumb to his finger according to a 3-digit number that appears on the screen.

2.1.Warm-up trial: Sessions 1-3 begin with a warm-up trial consisted of 2 cycles. Each cycle includes watch (60 seconds), regulate (90 seconds) and washout (30 seconds). Upon completion of the warmup the participants are asked about the strategies they employed and how well these were perceived to affect the scenario. The purpose of the warm-up trial is to ensure that participant comprehends the instructions and feels comfortable in continuing the training.

2.2.NF training trail: conducted through the training period (session 1-6) and consists 5 cycles including watch (60 seconds), regulate (60 seconds) and washout (30 seconds). Upon completion of the training the trainer interviews the participant about their scene of success and documents the strategies that were perceived as beneficial.

2.3.No-Feedback trial: IThe no-feedback trial aims to test learning sustainability in the absence of online feedback. It is structured similarly to the regular training (Two cycles of watch [60 seconds], regulate [60 seconds] and washout [30 seconds]) except that during regulate the scenario does not change online but is rather fixed on 75% agitation. The participant is instructed to employ the same mental strategies he or she found beneficial at the regular training. Following each cycle the participants receive feedback from the trainer regarding their level of success.

2.4.Cognitive-Interference trail: To further train to down-regulate the amygdala while engaged in an additional cognitive task, upon completion of NF training in sessions 5-6 participants conduct a “cognitive-interference” trial during which participants are instructed to down-regulate the relevant brain signal while conducting a simultaneous memory task. The interference task consisted of a single cycle, including one watch condition (60 sec) and one regulate condition (120 sec). While regulating the targeted signal participants are instructed to memorize as many details as possible from the animated scenario (positioning of different characters, clothing, objects etc.). After the completion of the NF trial (watch and regulation conditions) participants were asked to answer a 13-item multiple choice questionnaire.

3. The Amygdala-EFP model: EEG data used for the model is a Time/Frequency matrix recorded from electrode Pz including all frequency bands in a sliding time window of 12 seconds. To obtain the amygdala BOLD predictor, the EEG data are multiplied by the EFP model coefficients matrix. The EFP model consists of a frequency by delay by weight matrix in which every frequency band is differently weighted in different time delays. One sampling unite, calculated every three seconds, contains weighted data from the last 12 seconds. While conventional EEG measures used for NF commonly calculate the amplitude of specific band-widths or the ratio between them, the Amyg-EFP takes into account the spectrum of 1-60 Hz in a time window of 12 seconds.

4. On line calculation of amygdala signal: Online calculation of the Amygdala-EFP amplitude is conducted via Brain Vision RecView software which makes it possible to remove cardio-ballistic artifacts from the EEG data in real time using a built-in automated implementation of the average artifact subtraction method. RecView™ was custom modified to enable export of the corrected EEG data in real time through a TCP/IP socket. Preprocessing algorithm and AMY-EFP calculation models are compiled from Matlab R2009b™ to Microsoft. NET™ in order to be executed within the Brain Vision RecView™ EEG Recorder system. Data is then transferred to a MATLAB.NET compiled DLL that calculated the value of the AMY-EFP amplitude every 3 seconds.

4.1. Calibration: Given the nature of the Amygdala-EFP model which takes into account a time window of 12 seconds, each trial begins with a calibration period of 24 seconds in which the subject views a fixation cross. This period insures reliable calculation of the Amygdala-EFP signal during the first ‘watch’ condition.

5. Animated Scenario feedback generation: The unrest level ranges between zero [0] (all characters are sitting down) and one [1] (all characters are standing up). During “watch” blocks the unrest level is pre-set to 0.75. During “regulate” blocks the unrest level is set in accordance to the momentary AMY-EFP value. Mathematically, the unrest level at time point t of the regulate block is determined by the probability (p-value) of the AMY-EFP/beta value received at time point t, under the AMY-EFP/beta distribution of the watch block.

$$Unrest ( t ) = p \{ \frac{Amygdala ( t ) - μ ( A m y g d a l {a}_{w a t c h} )}{σ ( A m y g d a l {a}_{w a t c h} )} \} Unrest ( t ) = p \{ \frac{Amygdala ( t ) - μ ( A m y g d a l {a}_{w a t c h} )}{σ ( A m y g d a l {a}_{w a t c h} )} \}$$

Amygdala (t) is the amygdala activity value (Either EFP or beta) at time point t, and μ(Amygdala_{watch}) is the mean amygdala activity value during the previous watch block. σ(Amygdala_{watch}) is the standard deviation of the amygdala activity distribution during watch. A matching soundtrack recorded inside a real hospital complements the system output. Three alternative soundtracks with different agitation levels are produced and switched according to the AMY-EFP index. The system is implemented using the Unreal Development Kit (UDK™) game engine, which controls walking animations for individual characters.

TABLE 1
Order and type of NF tasks conducted at each session

| | NeuroFeedback | NeuroFeedback | No- | Cognitive- |
|---|---|---|---|---|
| | Warm-up | Training | Feedback | Interference |
| | (2 Cycels, | (5 Cycels, | (2 Cyces, | (1 Cycle, |
| | 6 min.) | 12:30 min.) | 3 min.) | 2 min.) |
| Session 1 | √ | √ | | |
| Session 2 | √ | √ | | |
| Session 3 | √ | √ | | |
| Session 4 | | √ | √ | |
| Session 5 | | √ | √ | √ |
| Session 6 | | √ | √ | √ |

### References for “Supplementary Methods and Results for the Exemplary Validation Experiment”

1. Paret, C. et al. fMRI neurofeedback of amygdala response to aversive stimuli enhances prefrontal—limbic brain connectivity. NeuroImage 125, 182-188 (2016).

2. Young, K. D. et al. Randomized Clinical Trial of Real-Time Fmri Amygdala Neurofeedback for Major Depressive Disorder: Effects on Symptoms and Autobiographical Memory Recall. Am. J. Psychiatry 174, 748-755 (2017).

3. Alegria, A. A. et al. Real-time fMRI neurofeedback in adolescents with attention deficit hyperactivity disorder. Hum. Brain Mapp. 38, 3190-3209 (2017).

4. Keynan, J. N. et al. Limbic Activity Modulation Guided by Functional Magnetic Resonance Imaging—Inspired Electroencephalography Improves Implicit Emotion Regulation. Biol. Psychiatry 80, 490-496 (2016).

5. Kober, S. E., Witte, M., Grinschgl, S., Neuper, C. & Wood, G. Placebo hampers ability to self-regulate brain activity: A double-blind sham-controlled neurofeedback study. NeuroImage (2018).

6. Gruzelier, J. H. EEG-neurofeedback for optimising performance. III: a review of methodological and theoretical considerations. Neurosci. Biobehay. Rev. 44, 159-182 (2014).

7. Gruzelier, J. H. EEG-neurofeedback for optimising performance. II: creativity, the performing arts and ecological validity. Neurosci. Biobehay. Rev. 44, 142-158 (2014).

8. Kinreich, S., Podlipsky, I., Intrator, N. & Hendler, T. Categorized EEG neurofeedback performance unveils simultaneous fMRI deep brain activation. Mach. Learn. Interpret. Neuroimaging 108-115 (2012).

9. Paret, C. et al. Alterations of amygdala-prefrontal connectivity with real-time fMRI neurofeedback in BPD patients. Soc. Cogn. Affect. Neurosci. 11, 952-960 (2016).

10. Zotev, V. et al. Real-time fMRI neurofeedback training of the amygdala activity with simultaneous EEG in veterans with combat-related PTSD. NeuroImage Clin. 19, 106-121 (2018).

11. Ros, T. et al. Optimizing microsurgical skills with EEG neurofeedback. BMC Neurosci. 10, 87 (2009).

12. Egner, T., Strawson, E. & Gruzelier, J. H. EEG Signature and Phenomenology of Alpha/theta Neurofeedback Training Versus Mock Feedback. Appl. Psychophysiol. Biofeedback 27, 261 (2002).

13. Jonassen, D. H. & Grabowski, B. L. Handbook of Individual Differences, Learning, and Instruction. (Routledge, 2012). doi:10.4324/9780203052860

It is expected that during the life of a patent maturing from this application many relevant methods and devices for measurement of EEG signals will be developed; the scope of the term EEG electrodes is intended to include all such new technologies a priori. As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for resilience training based on neurofeedback control of a selected deeply located limbic brain area, comprising:
(a) providing a reference EEG signature indicating activity and/or a change in activity of at least one selected deeply located limbic brain area;
(b) exposing a healthy human subject to one or more stress-evoking perturbations selected to affect activation of said at least one selected deeply located limbic brain area;
(c) instructing said healthy human subject to perform in a timed relation to said exposing, at least one activity configured to selectively affect activation of said at least one selected deeply located limbic brain area;
(d) recording EEG signals from said healthy human subject during said exposing;
(e) analyzing said recorded EEG signals using said reference EEG signature to determine an activation level of said at least one selected deeply located limbic brain area;
(f) delivering a human-detectable indication to said healthy human subject according to said determined activation level;
repeating said (b) to (f) to increase a resilience of said healthy human subject.

2. A method according to claim 1, wherein said at least one activity comprises at least one mental activity or at least one physical activity.

3. A method according to claim 1, wherein said recording comprises recording EEG signals from said healthy human subject during and/or following the performing of said one or more activities, and wherein said determine comprises determining an activation level and/or a change in activation level of said at least one selected deeply located limbic brain area based on an identified relation between said reference EEG signature indicating said activation level and/or said change in activation level and said analyzed recorded EEG signals.

4. A method according to claim 1, wherein said delivering comprises modifying said one or more stress-evoking perturbations according to said determined activation level.

5. A method according to claim 1, comprising selecting said at least one activity out of two or more activities based on an ability of said at least one activity to selectively affect activation of said at least one selected deeply located limbic brain area when performed by said healthy human subject.

6. A method according to claim 1, wherein said one or more stress-evoking perturbations are perturbations selected to induce a stress response in said healthy human subject.

7. A method according to claim 1, wherein said at least one selected deeply located limbic brain area, is a brain region related to the limbic system located underneath the brain cortex.

8. A method according to claim 1, wherein said at least one selected deeply located limbic area comprises an amygdala.

9. A method according to claim 1, wherein said timed relation comprises prior-to, during and/or after said exposing.

10. A method according to claim 1, wherein said at least one activity activates brain regions or neural circuits which relate to activation control of said at least one selected deeply located limbic brain area.

11. A method according to claim 1, comprising identifying a relation between said analyzed recorded EEG signals and said provided EEG signature, and wherein said activation level of said at least one selected deeply located limbic brain area is determined based on said identified relation.

12. A method according to claim 11, wherein said reference EEG signature comprises a fMRI-inspired EEG model generated by calculating a correlation between one or more measured EEG signals, and an activity level of said at least one selected deeply located limbic brain area as monitored by fMRI.

13. A method according to claim 1, comprising delivering an indication regarding a resilience of said healthy subject based on a change in said determined activation level of said at least one selected deeply located limbic brain area following performing of said at least one activity, wherein said resilience is an ability of said healthy subject to resist and/or overcome deleterious short- and or long-term effects associated with a stressor.

14. A resilience training system, comprising:
a user interface;
one or more electrodes configured to measure EEG signals;
a memory for storing at least one reference EEG signature indicating an activity level and/or a change in activity level of at least one selected deeply located limbic brain area;
a control unit electrically connected to said memory, user interface and to said one or more electrodes, wherein said control unit is configured to:
(a) display an interface to a subject by said user interface, wherein said interface follows an activity level of said at least one selected deeply located limbic brain area of said subject;
(b) provide instructions using said user interface to said subject how to modulate activity of said at least one selected deeply located limbic brain area;
(c) record EEG signals from said subject by said one or more electrodes;
(d) analyze said recorded EEG signals to identify a relation between said analyzed recorded EEG signals and said stored reference EEG signature indicating an activity level of said at least one selected deeply located limbic brain area;
(e) determine an activity level of said at least one selected deeply located limbic brain area based on said determined relation;
(e) modify said interface according to said determined activity level.

15. A system according to claim 14, wherein said control unit is configured to calculate a resilience score indicating an ability of said subject to modulate an activity of said at least one selected deeply located limbic brain area, based on said determined activity.

16. A system according to claim 14, wherein said control unit is configured to modify said instructions delivered by said user interface according to said determined activity.

17. A system according to claim 14, wherein said control unit is configured to display said interface and/or to provide said instructions according to an alexithymia level or indication thereof stored in said memory.

18. A system according to claim 14, wherein said control unit is configured to display said interface and/or to provide said instructions according to a quantified learning model of said subject or indication thereof stored in said memory.

19. A system according to claim 14, wherein said at least one selected deeply located limbic brain area comprises an amygdala.

20. A system according to claim 14, wherein said stored reference EEG signature comprises a fMRI-inspired EEG model generated by calculating a correlation between one or more measured EEG signals, and an activity level of said at least one selected deeply located limbic brain area as monitored by fMRI.
